SEQUENCE LISTING (456 sequences)

SEQ ID NO: 1 - protein, 218 aa, synthetic construct
  REGION 1..218: note = chemically synthesized
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, synthetic construct
  REGION 1..215: note = chemically synthesized
PAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 217 aa, synthetic construct
  REGION 1..217: note = chemically synthesized
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 218 aa, synthetic construct
  REGION 1..218: note = chemically synthesized
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 218 aa, synthetic construct
  REGION 1..218: note = chemically synthesized
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 218 aa, synthetic construct
  REGION 1..218: note = chemically synthesized
PAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 14 aa, synthetic construct
  REGION 1..14: note = chemically synthesized
EPKSSDKTHTCPPC

SEQ ID NO: 8 - protein, 9 aa, synthetic construct
  REGION 1..9: note = chemically synthesized
DKTHTCPPC

SEQ ID NO: 9 - protein, 11 aa, synthetic construct
  REGION 1..11: note = chemically synthesized
ESKYGPPCPPC

SEQ ID NO: 10 - protein, 6 aa, synthetic construct
  REGION 1..6: note = chemically synthesized
GGSGGS

SEQ ID NO: 11 - protein, 9 aa, synthetic construct
  REGION 1..9: note = chemically synthesized
GGSGGSGGS

SEQ ID NO: 12 - protein, 12 aa, synthetic construct
  REGION 1..12: note = chemically synthesized
GGSGGSGGSGGS

SEQ ID NO: 13 - protein, 15 aa, synthetic construct
  REGION 1..15: note = chemically synthesized
GGSGGSGGSGGSGGS

SEQ ID NO: 14 - protein, 12 aa, synthetic construct
  REGION 1..12: note = chemically synthesized
GQGTLVTVKPGG

SEQ ID NO: 15 - protein, 12 aa, synthetic construct
  REGION 1..12: note = chemically synthesized
GQGTLVTVEPGG

SEQ ID NO: 16 - protein, 123 aa, synthetic construct
  REGION 1..123: note = chemically synthesized
QVQLQESGGGLVQAGDSLRLSCAASGWAFDNYGMAWFRQAPGKEREFIGRLAWNGGSTDYADSVKGRFTISRDNPKNTLYLQMNNLKPEDTAVYYCARQRSYSGYGIRTPQTYDYWGQGTQVT

SEQ ID NO: 17 - protein, 8 aa, synthetic construct
  REGION 1..8: note = chemically synthesized
GWAFDNYG

SEQ ID NO: 18 - protein, 8 aa, synthetic construct
  REGION 1..8: note = chemically synthesized
LAWNGGST

SEQ ID NO: 19 - protein, 19 aa, synthetic construct
  REGION 1..19: note = chemically synthesized
ARQRSYSGYGIRTPQTYDY

SEQ ID NO: 20 - protein, 123 aa, synthetic construct
  REGION 1..123: note = chemically synthesized
QVQLQQSGGGLVQAGDSLRLSCAASGWAFGNYGMAWFRRAPGKEREFIGRLAWNGGSTDYVDSVKGRFTISRDNPKNTLYLQMNNLKPDDTAVYYCARQRSYSRYDIRTPQTYDYWGQGTQVT

SEQ ID NO: 21 - protein, 8 aa, synthetic construct
  REGION 1..8: note = chemically synthesized
GWAFGNYG

SEQ ID NO: 22 - protein, 19 aa, synthetic construct
  REGION 1..19: note = chemically synthesized
ARQRSYSRYDIRTPQTYDY

SEQ ID NO: 23 - protein, 123 aa, synthetic construct
  REGION 1..123: note = chemically synthesized
QVQLVQSGGGLVQPGGSLRLSCAASGWAFDNYGMAWFRQAPGKEREFIGRLAWNGGSTDYADSVKGRFTISRDNPKNTLYLQMNSLKPEDTAVYYCARQRSYSRYGIRAPQTYDYWGQGTQVT

SEQ ID NO: 24 - protein, 19 aa, synthetic construct
  REGION 1..19: note = chemically synthesized
ARQRSYSRYGIRAPQTYDY

SEQ ID NO: 25 - protein, 118 aa, synthetic construct
  REGION 1..118: note = chemically synthesized
QVQLQESGGGLVQPGGSLRLSCAVSGFSFSINAMGWYRQAPGKRREFLAAIDSGRNTVYAVSVKGRFTISRDNAKNTVYLQMNSLKPEDTAIYYCGLLKGNRVVSPSVAYWGQGTQVT

SEQ ID NO: 26 - protein, 8 aa, synthetic construct
  REGION 1..8: note = chemically synthesized
GFSFSINA

SEQ ID NO: 27 - protein, 7 aa, synthetic construct
  REGION 1..7: note = chemically synthesized
IDSGRNT

SEQ ID NO: 28 - protein, 15 aa, synthetic construct
  REGION 1..15: note = chemically synthesized
GLLKGNRVVSPSVAY

SEQ ID NO: 29 - protein, 120 aa, synthetic construct
  REGION 1..120: note = chemically synthesized
EVQPVQSGGGLVQAGESLRLSCAASATIFSNNAMGWYRQAPGKQRELVATITTGGFTNYRDSVKGRFDISRDNAKNTVYLQMNNLKPEDTAVYYCNVVLRYSRDYSYTTVKEYWGQGTQV

SEQ ID NO: 30 - protein, 8 aa, synthetic construct
  REGION 1..8: note = chemically synthesized
ATIFSNNA

SEQ ID NO: 31 - protein, 7 aa, synthetic construct
  REGION 1..7: note = chemically synthesized
ITTGGFT

SEQ ID NO: 32 - protein, 18 aa, synthetic construct
  REGION 1..18: note = chemically synthesized
NVVLRYSRDYSYTTVKEY

SEQ ID NO: 33 - protein, 126 aa, synthetic construct
  REGION 1..126: note = chemically synthesized
EVQLLESGGGEVQPGGSLRLSCAASGWAFGNYGMAWFRQAPGKGLEWVARLAWNGGSTDYAESVKGRFTISRDNAKNTLYLQMSSLRAEDTAVYYCARQRSYSRYDIRTPQTYDYWGQGTLVTVKP

SEQ ID NO: 34 - protein, 126 aa, synthetic construct
  REGION 1..126: note = chemically synthesized
EVQLLESGGGEVQPGGSLRLSCAASGWAFGNYGMAWFRQAPGKGREFVARLAWNGGSTDYAESVKGRFTISRDNAKNTLYLQMSSLRAEDTAVYYCARQRSYSRYDIRTPQTYDYWGQGTLVTVKP

SEQ ID NO: 35 - protein, 126 aa, synthetic construct
  REGION 1..126: note = chemically synthesized
EVQLLESGGGEVQPGGSLRLSCAASGWAFGNYGMAWFRQAPGKEREFVSRLAWNGGSTDYVAESVKGRFTISRDNAKNTLYLQMSSLRAEDTAVYYCARQRSYSRYDIRTPQTYDYWGQGTLVTVK

SEQ ID NO: 36 - protein, 126 aa, synthetic construct
  REGION 1..126: note = chemically synthesized
EVQLLESGGGEVQPGGSLRLSCAASGWAFGNYGMAWFRQAPGKEREFIGRLAWNGGSTDYVESVKGRFTISRDNPKNTLYLQMSSLRAEDTAVYYCARQRSYSRYDIRTPQTYDYWGQGTLVTVKP

SEQ ID NO: 37 - protein, 126 aa, synthetic construct
  REGION 1..126: note = chemically synthesized
EVQLLESGGGEVQPGGSLRLSCAASGWAFGNYGMAWFRQAPGKEREFVSRLAWNGGSTDYVESVKGRFTISRDNAKNTLYLQMSSLRAEDTAVYYCARQRSYSRYDIRTPQTYDYWGQGTLVTVKP

SEQ ID NO: 38 - protein, 126 aa, synthetic construct
  REGION 1..126: note = chemically synthesized
EVQLLESGGGEVQPGGSLRLSCAASGWAFGNYGMAWFRQAPGKEREFVSRLAWNGGSTDYVESVKGRFTISRDNPKNTLYLQMSSLRAEDTAVYYCARQRSYSRYDIRTPQTYDYWGQGTLVTVKP

SEQ ID NO: 39 - protein, 126 aa, synthetic construct
  REGION 1..126: note = chemically synthesized
EVQLLESGGGEVQPGGSLRLSCAASGWAFGNYGMAWFRQAPGKEREFIGRLAWNGGSTDYVESVKGRFTISRDNAKNTLYLQMSSLRAEDTAVYYCARQRSYSRYDIRTPQTYDYWGQGTLVTVKP

SEQ ID NO: 40 - protein, 126 aa, synthetic construct; identical to SEQ ID NO: 36
  REGION 1..126: note = chemically synthesized

SEQ ID NO: 41 - protein, 126 aa, synthetic construct
  REGION 1..126: note = chemically synthesized
EVQLLESGGGEVQPGGSLRLSCAASGWAFGNYGMAWFRQAPGKEREFIGRLAWQGGSTDYVESVKGRFTISRDNPKNTLYLQMSSLRAEDTAVYYCARQRSYSRYDIRTPQTYDYWGQGTLVTVKP

SEQ ID NO: 42 - protein, 8 aa, synthetic construct
  REGION 1..8: note = chemically synthesized
LAWQGGST

SEQ ID NO: 43 - protein, 126 aa, synthetic construct
  REGION 1..126: note = chemically synthesized
EVQLLESGGGEVQPGGSLRLSCAASGWAFGNYGMAWFRQAPGKEREFIGRLAWNAGSTDYVESVKGRFTISRDNPKNTLYLQMSSLRAEDTAVYYCARQRSYSRYDIRTPQTYDYWGQGTLVTVKP

SEQ ID NO: 44 - protein, 8 aa, synthetic construct
  REGION 1..8: note = chemically synthesized
LAWNAGST

SEQ ID NO: 45 - protein, 126 aa, synthetic construct
  REGION 1..126: note = chemically synthesized
EVQLLESGGGEVQPGGSLRLSCAASGWAFGNYGMAWFRQAPGKEREFVSRLAWQGGSTDYVESVKGRFTISRDNAKNTLYLQMSSLRAEDTAVYYCARQRSYSRYDIRTPQTYDYWGQGTLVTVKP

SEQ ID NO: 46 - protein, 126 aa, synthetic construct
  REGION 1..126: note = chemically synthesized
EVQLLESGGGEVQPGGSLRLSCAASGWAFGNYGMAWFRQAPGKEREFVSRLAWNAGSTDYVESVKGRFTISRDNAKNTLYLQMSSLRAEDTAVYYCARQRSYSRYDIRTPQTYDYWGQGTLVTVKP

SEQ ID NO: 47 - protein, 126 aa, synthetic construct
  REGION 1..126: note = chemically synthesized
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKP

SEQ ID NO: 48 - protein, 8 aa, synthetic construct
  REGION 1..8: note = chemically synthesized
LAWGGGST

SEQ ID NO: 49 - protein, 126 aa, synthetic construct
  REGION 1..126: note = chemically synthesized
EVQLLESGGGEVQPGGSLRLSCAASGWAFSNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKP

SEQ ID NO: 50 - protein, 8 aa, synthetic construct
  REGION 1..8: note = chemically synthesized
GWAFSNYG

SEQ ID NO: 51 - protein, 126 aa, synthetic construct
  REGION 1..126: note = chemically synthesized
EVQLLESGGGEVQPGGSLRLSCAASGWAFGNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKP

SEQ ID NO: 52 - protein, 8 aa, synthetic construct
  REGION 1..8: note = chemically synthesized
LAWSGGST

SEQ ID NO: 53 - protein, 126 aa, synthetic construct
  REGION 1..126: note = chemically synthesized
EVQLLESGGGEVQPGGSLRLSCAASGWAFSNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKP

SEQ ID NO: 54 - protein, 126 aa, synthetic construct
  REGION 1..126: note = chemically synthesized
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSGYDIRTPQTYDYWGQGTLVTVKP

SEQ ID NO: 55 - protein, 19 aa, synthetic construct
  REGION 1..19: note = chemically synthesized
ARQRSYSGYDIRTPQTYDY

SEQ ID NO: 56 - protein, 126 aa, synthetic construct
  REGION 1..126: note = chemically synthesized
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSRYGIRTPQTYDYWGQGTLVTVKP

SEQ ID NO: 57 - protein, 19 aa, synthetic construct
  REGION 1..19: note = chemically synthesized
ARQRSYSRYGIRTPQTYDY

SEQ ID NO: 58 - protein, 126 aa, synthetic construct
  REGION 1..126: note = chemically synthesized
EVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSGYGIRTPQTYDYWGQGTLVTVKP

SEQ ID NO: 59 - protein, 121 aa, synthetic construct
  REGION 1..121: note = chemically synthesized
EVQLLESGGGEVQPGGSLRLSCAASGFSFSINAMGWYRQAPGKGLEWVAAIDSGRNTVYAESVKGRFTISRDNAKNTLYLQMSSLRAEDTAVYYCGLLKGNRVVSPSVAYWGQGTLVTVKP

SEQ ID NO: 60 - protein, 121 aa, synthetic construct
  REGION 1..121: note = chemically synthesized
EVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 61 - protein, 7 aa, synthetic construct
  REGION 1..7: note = chemically synthesized
IESGRNT

SEQ ID NO: 62 - protein, 121 aa, synthetic construct
  REGION 1..121: note = chemically synthesized
EVQLLESGGGEVQPGGSLRLSCAASGFSFSINAMGWYRQAPGKRREFVAAIYSGRNTVYAESVKGRFTISRDNAKNTVYLQMSSLRAEDTAVYYCGLLKGNRVVSPSVAYWGQGTLVTVKP

SEQ ID NO: 63 - protein, 7 aa, synthetic construct
  REGION 1..7: note = chemically synthesized
IYSGRNT

SEQ ID NO: 64 - protein, 121 aa, synthetic construct
  REGION 1..121: note = chemically synthesized
EVQLLESGGGEVQPGGSLRLSCAASGFTFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 65 - protein, 8 aa, synthetic construct
  REGION 1..8: note = chemically synthesized
GFTFSINA

SEQ ID NO: 66 - protein, 121 aa, synthetic construct
  REGION 1..121: note = chemically synthesized
EVQLLESGGGEVQPGGSLRLSCAASGFSFSINAMSWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 67 - protein, 8 aa, synthetic construct
  REGION 1..8: note = chemically synthesized
GFSFSINA

SEQ ID NO: 68 - protein, 121 aa, synthetic construct
  REGION 1..121: note = chemically synthesized
EVQLLESGGGEVQPGGSLRLSCAASGFTFSSNAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 69 - protein, 8 aa, synthetic construct
  REGION 1..8: note = chemically synthesized
GFTFSSNA

SEQ ID NO: 70 - protein, 121 aa, synthetic construct
  REGION 1..121: note = chemically synthesized
EVQLLESGGGEVQPGGSLRLSCAASGFSFSINAMGWYRQAPGKRREFVAAIESSRNTVYAESVKGRFTISRDNAKNTVYLQMSSLRAEDTAVYYCGLLKGNRVVSPSVAYWGQGTLVTVKP

SEQ ID NO: 71 - protein, 7 aa, synthetic construct
  REGION 1..7: note = chemically synthesized
IESSRNT

SEQ ID NO: 72 - protein, 121 aa, synthetic construct
  REGION 1..121: note = chemically synthesized
EVQLLESGGGEVQPGGSLRLSCAASGFSFSINAMGWYRQAPGKRREFVAAIESGSNTVYAESVKGRFTISRDNAKNTVYLQMSSLRAEDTAVYYCGLLKGNRVVSPSVAYWGQGTLVTVKP

SEQ ID NO: 73 - protein, 7 aa, synthetic construct
  REGION 1..7: note = chemically synthesized
IESGSNT

SEQ ID NO: 74 - protein, 121 aa, synthetic construct
  REGION 1..121: note = chemically synthesized
EVQLLESGGGEVQPGGSLRLSCAASGFSFSINAMGWYRQAPGKRREFVAAIESGRSTVYAESVKGRFTISRDNAKNTVYLQMSSLRAEDTAVYYCGLLKGNRVVSPSVAYWGQGTLVTVKP

SEQ ID NO: 75 - protein, 7 aa, synthetic construct
  REGION 1..7: note = chemically synthesized
IESGRST

SEQ ID NO: 76 - protein, 121 aa, synthetic construct
  REGION 1..121: note = chemically synthesized
EVQLLESGGGEVQPGGSLRLSCAASGFSFSINAMGWYRQAPGKRREFVAAIESGRNTYYAESVKGRFTISRDNAKNTVYLQMSSLRAEDTAVYYCGLLKGNRVVSPSVAYWGQGTLVTVKP

SEQ ID NO: 77 - protein, 7 aa, synthetic construct
  REGION 1..7: note = chemically synthesized
IESGRNT

SEQ ID NO: 78 - protein, 121 aa, synthetic construct
  REGION 1..121: note = chemically synthesized
EVQLLESGGGEVQPGGSLRLSCAASGFSFSINAMGWYRQAPGKRREFVAAIYSGSSTVYAESVKGRFTISRDNAKNTVYLQMSSLRAEDTAVYYCGLLKGNRVVSPSVAYWGQGTLVTVKP

SEQ ID NO: 79 - protein, 7 aa, synthetic construct
  REGION 1..7: note = chemically synthesized
IYSGSST

SEQ ID NO: 80 - protein, 121 aa, synthetic construct
  REGION 1..121: note = chemically synthesized
EVQLLESGGGEVQPGGSLRLSCAVSGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKP

SEQ ID NO: 81 - protein, 121 aa, synthetic construct
  REGION 1..121: note = chemically synthesized
EVQLLESGGGEVQPGGSLRLSCAASGFSFSINAMGWYRQAPGKRREFVAAIESGRNTVYAVSVKGRFTISRDNAKNTVYLQMSSLRAEDTAVYYCGLLKGNRVVSPSVAYWGQGTLVTVKP

SEQ ID NO: 82 - protein, 121 aa, synthetic construct
  REGION 1..121: note = chemically synthesized
EVQLLESGGGEVQPGGSLRLSCAASGFSFSINAMGWYRQAPGKGREFVAAIESGRNTVYAESVKGRFTISRDNAKNTVYLQMSSLRAEDTAVYYCGLLKGNRVVSPSVAYWGQGTLVTVKP

SEQ ID NO: 83 - protein, 121 aa, synthetic construct
  REGION 1..121: note = chemically synthesized
EVQLLESGGGEVQPGGSLRLSCAASGFSFSINAMGWYRQAPGKRREFLAAIESGRNTVYAESVKGRFTISRDNAKNTVYLQMSSLRAEDTAVYYCGLLKGNRVVSPSVAYWGQGTLVTVKP

SEQ ID NO: 84 - protein, 119 aa, synthetic construct
  REGION 1..119: note = chemically synthesized
QVQLVQSGAEVKKPGSSVKVSCKASGGTFNSYAISWVRQAPGQGLEWMGGIIPGFGTANYAQKFQGRVTITADESTSTAYMELSSLRSEDTAVYYCARKNEEDGGFDHWGQGTLVTVSS

SEQ ID NO: 85 - protein, 118 aa, synthetic construct
  REGION 1..118: note = chemically synthesized
QVQLVESGGGLVQPGGSLRLSCAASGFTFSDYYMHWVRQAPGKGLEWVSVISGSGSNTYYADSVKGRFTISRDNSKNTLYLQMNSLRAEDTAVYYCARLYAQFEGDFWGQGTLVTVSS

SEQ ID NO: 86 - protein, 116 aa, synthetic construct
  REGION 1..116: note = chemically synthesized
QVQLVQSGAEVKKPGESLKISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 87 - protein, 116 aa, synthetic construct
  REGION 1..116: note = chemically synthesized
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 88 - protein, 107 aa, synthetic construct
  REGION 1..107: note = chemically synthesized
DIELTQPPSVSVAPGQTARISCSGDNLGDYYASWYQQKPGQAPVLVIYDDSNRPSGIPERFSGSNSGNTATLTISGTQAEDEADYYCQTWDGTLHFVFGGGTKLTVL

SEQ ID NO: 89 - protein, 107 aa, synthetic construct
  REGION 1..107: note = chemically synthesized
DIELTQPPSVSVAPGQTARISCSGDNIGSKYVSWYQQKPGQAPVLVIYSDSERPSGIPERFSGSNSGNTATLTISGTQAEDEADYYCQSWDGSISRVFGGGTKLTVL

SEQ ID NO: 90 - protein, 108 aa, synthetic construct
  REGION 1..108: note = chemically synthesized
DIELTQPPSVSVAPGQTARISCSGDNIGDQYAHWYQQKPGQAPVVVIYQDKNRPSGIPERFSGSNSGNTATLTISGTQAEDEADYYCATYTGFGSLAVFGGGTKLTVL

SEQ ID NO: 91 - protein, 108 aa, synthetic construct
  REGION 1..108: note = chemically synthesized
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 92 - protein, 108 aa, synthetic construct
  REGION 1..108: note = chemically synthesized
SYELTQPPSVSVSPGQTASITCSGDNIGDQYAHWYQQKPGQSPVVVIYQDKNRPSGIPERFSGSNSGNTATLTISGTQAMDEADYYCATYTGFGSLAVFGGGTKLTVL

SEQ ID NO: 93 - protein, 108 aa, synthetic construct
  REGION 1..108: note = chemically synthesized
DIELTQPPSVSVAPGQTARISCSGDNIGDQYAHWYQQKPGQAPVVVIYQDKNRPSGIPERFSGSNSGNTATLTISGTQAEDEADYYCSTYTFVGFTTVFGGGTKLTVL

SEQ ID NO: 94 - protein, 108 aa, synthetic construct
  REGION 1..108: note = chemically synthesized
SYELTQPPSVSVSPGQTASITCSGDNIGDQYAHWYQQKPGQSPVLVIYQDKNRPSGIPERFSGSNSGNTATLTISGTQAMDEADYYCSTYTFVGFTTVFGGGTKLTVL

SEQ ID NO: 95 - protein, 108 aa, synthetic construct
  REGION 1..108: note = chemically synthesized
SYELTQPPSVSVSPGQTASITCSGDNIGDQYAHWYQQKPGQSPVVVIYQDKNRPSGIPERFSGSNSGNTATLTISGTQAMDEADYYCSTYTFVGFTTVFGGGTKLTVL

SEQ ID NO: 96 - protein, 448 aa, synthetic construct
  REGION 1..448: note = chemically synthesized
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 451 aa, synthetic construct
  REGION 1..451: note = chemically synthesized
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 216 aa, synthetic construct
  REGION 1..216: note = chemically synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 178 aa, synthetic construct
  REGION 1..178: note = chemically synthesized
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 100 - protein, 109 aa, synthetic construct
  REGION 1..109: note = chemically synthesized
QVQLQESGGGLVQAGGSLRLACTTSGGIFNIRPISWYRQPPGMQREWVATIAFGGATNYANSIKGRFTASRDNAKNTVYLQMNGLKPEDTAVYYCNAFEIWGQGTQVTV

SEQ ID NO: 101 - protein, 8 aa, synthetic construct
  REGION 1..8: note = chemically synthesized
GGIFNIRP

SEQ ID NO: 102 - protein, 7 aa, synthetic construct
  REGION 1..7: note = chemically synthesized
IAFGGAT

SEQ ID NO: 103 - protein, 5 aa, synthetic construct
  REGION 1..5: note = chemically synthesized
NAFEI

SEQ ID NO: 104 - protein, 109 aa, synthetic construct
  REGION 1..109: note = chemically synthesized
QLQLQESGGGLVRAGGSLRLACTTSGGIFAIKPISWYRQPPGQEREWVTTTTSSGATNYANSIKGRFTVARDNAKNTVYLQMNDLKLEDTAVYYCNVFEYWGQGTQVTV

SEQ ID NO: 105 - protein, 8 aa, synthetic construct
  REGION 1..8: note = chemically synthesized
GGIFAIKP

SEQ ID NO: 106 - protein, 7 aa, synthetic construct
  REGION 1..7: note = chemically synthesized
TTSSGAT

SEQ ID NO: 107 - protein, 5 aa, synthetic construct
  REGION 1..5: note = chemically synthesized
NVFEY

SEQ ID NO: 108 - protein, 109 aa, synthetic construct
  REGION 1..109: note = chemically synthesized
QVQLQESGGDLVQAGSSLRLACATSGGVFNIRPISWYRQPPGKQREWVATIASGGATNYANSIKGRFTASRDNAKNTVYLQMNGLKPEDTAVYYCNAFEVWGQGTQVTV

SEQ ID NO: 109 - protein, 8 aa, synthetic construct
  REGION 1..8: note = chemically synthesized
GGVFNIRP

SEQ ID NO: 110 - protein, 7 aa, synthetic construct
  REGION 1..7: note = chemically synthesized
IASGGAT

SEQ ID NO: 111 - protein, 5 aa, synthetic construct
  REGION 1..5: note = chemically synthesized
NAFEV

SEQ ID NO: 112 - protein, 109 aa, synthetic construct
  REGION 1..109: note = chemically synthesized
QVQLQQSGGGLVQAGGSLRLACTTSGGIFNIRPISWYRQPPGMQREWVATIASGGATNYANSIKGRFTASRDNAKNTVYLQMNGLKPEDTAVYYCNTLNFWGRGTQVTV

SEQ ID NO: 113 - protein, 5 aa, synthetic construct
  REGION 1..5: note = chemically synthesized
NTLNF

SEQ ID NO: 114 - protein, 109 aa, synthetic construct
  REGION 1..109: note = chemically synthesized
QVQLQESGGGLVQAGGSLRLACTTSGGIFNIRPISWYRQPPGMQREWVATIASGGATNYANSIKGRFTASRDNAKNTVYLQMNGLKPEDTAVYYCNVFEIWGQGTQVTV

SEQ ID NO: 115 - protein, 5 aa, synthetic construct
  REGION 1..5: note = chemically synthesized
NVFEI

SEQ ID NO: 116 - protein, 109 aa, synthetic construct
  REGION 1..109: note = chemically synthesized
QVQLQQSGGGLVQAGGSLRLACITSGGIFNIRPISWYRQPPGKQREWVATIASGGAANYANSIKGRFTASRDNAKNTVYLQMNGLKPEDTAVYYCNAFENWGQGTQVTV

SEQ ID NO: 117 - protein, 7 aa, synthetic construct
  REGION 1..7: note = chemically synthesized
IASGGAA

SEQ ID NO: 118 - protein, 5 aa, synthetic construct
  REGION 1..5: note = chemically synthesized
NAFEN

SEQ ID NO: 119 - protein, 111 aa, synthetic construct
  REGION 1..111: note = chemically synthesized
QVQLQESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKP

SEQ ID NO: 120 - protein, 111 aa, synthetic construct
  REGION 1..111: note = chemically synthesized
EVQLQESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKP

SEQ ID NO: 121 - protein, 111 aa, synthetic construct
  REGION 1..111: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKP

SEQ ID NO: 122 - protein, 111 aa, synthetic construct; identical to SEQ ID NO: 121
  REGION 1..111: note = chemically synthesized

SEQ ID NO: 123 - protein, 111 aa, synthetic construct
  REGION 1..111: note = chemically synthesized
EVQLLESGGGEVQPGGSLRLSCAASGGIFAIKPISWYRQAPGKQREWVTTTTSSGATNYAESVKGRFTISRDNAKNTVYLQMSSLRAEDTAVYYCNVFEYWGQGTLVTVKP

SEQ ID NO: 124 - protein, 111 aa, synthetic construct; identical to SEQ ID NO: 121
  REGION 1..111: note = chemically synthesized

SEQ ID NO: 125 - protein, 111 aa, synthetic construct
  REGION 1..111: note = chemically synthesized
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVT

SEQ ID NO: 126 - protein, 117 aa, synthetic construct
  REGION 1..117: note = chemically synthesized
QVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSS

SEQ ID NO: 127 - protein, 123 aa, synthetic construct
  REGION 1..123: note = chemically synthesized
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 128 - protein, 112 aa, synthetic construct
  REGION 1..112: note = chemically synthesized
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGT

SEQ ID NO: 129 - protein, 120 aa, synthetic construct
  REGION 1..120: note = chemically synthesized
QVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSS

SEQ ID NO: 130 - protein, 110 aa, synthetic construct
  REGION 1..110: note = chemically synthesized
EVQLVESGGGLVQPGRSLRLSCAVSGFTFDDYVVHWVRQAPGKGLEWVSGNSGNIGYADSVKGRFTISRDNAKNSLYLQMNSLRAEDTALYYCAVPFDYWGQGTLVTVSS

SEQ ID NO: 131 - protein, 123 aa, synthetic construct
  REGION 1..123: note = chemically synthesized
QVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSS

SEQ ID NO: 132 - protein, 123 aa, synthetic construct
  REGION 1..123: note = chemically synthesized
QVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSS

SEQ ID NO: 133 - protein, 121 aa, synthetic construct
  REGION 1..121: note = chemically synthesized
QVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSS

SEQ ID NO: 134 - protein, 123 aa, synthetic construct
  REGION 1..123: note = chemically synthesized
QVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSS

SEQ ID NO: 135 - protein, 121 aa, synthetic construct
  REGION 1..121: note = chemically synthesized
EVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSS

SEQ ID NO: 136 - protein, 120 aa, synthetic construct
  REGION 1..120: note = chemically synthesized
QMQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARDYFWSGFSAFDIWGKGTLVTVS

SEQ ID NO: 137 - protein, 107 aa, synthetic construct
  REGION 1..107: note = chemically synthesized
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIK

SEQ ID NO: 138 - protein, 106 aa, synthetic construct
  REGION 1..106: note = chemically synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 139 - protein, 107 aa, synthetic construct
  REGION 1..107: note = chemically synthesized
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 140 - protein, 108 aa, synthetic construct
  REGION 1..108: note = chemically synthesized
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 141 - protein, 106 aa, synthetic construct
  REGION 1..106: note = chemically synthesized
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIK

SEQ ID NO: 142 - protein, 106 aa, synthetic construct
  REGION 1..106: note = chemically synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIK

SEQ ID NO: 143 - protein, 107 aa, synthetic construct
  REGION 1..107: note = chemically synthesized
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 144 - protein, 107 aa, synthetic construct
  REGION 1..107: note = chemically synthesized
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIK

SEQ ID NO: 145 - protein, 121 aa, synthetic construct
  REGION 1..121: note = chemically synthesized
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 146 - protein, 108 aa, synthetic construct
  REGION 1..108: note = chemically synthesized
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 147 - protein, 118 aa, synthetic construct
  REGION 1..118: note = chemically synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 148 - protein, 118 aa, synthetic construct
  REGION 1..118: note = chemically synthesized
EVQLVESGGGLVQPGGSLRLSCAASGFTFSGSWIHWVRQAPGKGLEWVAWILPYGGSSYYADSVKGRFTISADTSKNTAYLQMNSLRAEDTAVYYCARRHWPGGFDYWGQGTLVTVSA

SEQ ID NO: 149 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 150 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYYNVPWTFGQGTKVEIKR

SEQ ID NO: 151 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYYAPPWTFGQGTKVEIKR

SEQ ID NO: 152 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYYTVPWTFGQGTKVEIKR

SEQ ID NO: 153 - protein, 108 aa, synthetic construct
  REGION 1..108: note = chemically synthesized
DIQMTQSPSSLSASVGDRVTITCRASQVINTFLAWYQQKPGKAPKLLIYSASTLASGVPSRFSGSGSGTDFTLTISSLQPEDFATYYCQQYYTVPRTFGQGTKVEIKR

SEQ ID NO: 154 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQGYGVPRTFGQGTKVEIKR

SEQ ID NO: 155 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYLFTPPTFGQGTKVEIKR

SEQ ID NO: 156 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYFITPTTFGQGTKVEIKR

SEQ ID NO: 157 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYYYTPPTFGQGTKVEIKR

SEQ ID NO: 158 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQFFYTPPTFGQGTKVEIKR

SEQ ID NO: 159 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQSLFTPPTFGQGTKVEIKR

SEQ ID NO: 160 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQSLYTPPTFGQGTKVEIKR

SEQ ID NO: 161 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQSWYHPPTFGQGTKVEIKR

SEQ ID NO: 162 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYFYIPPTFGQGTKVEIKR

SEQ ID NO: 163 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYWYTPTTFGQGTKVEIKR

SEQ ID NO: 164 - protein, 108 aa, synthetic construct
  REGION 1..108: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQSYFIPPTFGQGTKVEIKR

SEQ ID NO: 165 - protein, 141 aa, synthetic construct
  REGION 1..141: note = chemically synthesized
METGLRWLLLVAVLKGVQCLSVEESGGRLVTPGTPLTLTCTASGFTITNYHMFWVRQAPGKGLEWIGVITSSGIGSSSTTYYATWAKGRFTISKTSTTVNLRITSPTTEDTATYFCARDYFTNTYYALDIWGPGTLVTVSS

SEQ ID NO: 166 - protein, 123 aa, synthetic construct; identical to SEQ ID NO: 127
  REGION 1..123: note = chemically synthesized

SEQ ID NO: 167 - protein, 118 aa, synthetic construct
  REGION 1..118: note = chemically synthesized
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 168 - protein, 120 aa, synthetic construct; identical to SEQ ID NO: 129
  REGION 1..120: note = chemically synthesized

SEQ ID NO: 169 - protein, 113 aa, synthetic construct
  REGION 1..113: note = chemically synthesized
EVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSS

SEQ ID NO: 170 - protein, 123 aa, synthetic construct; identical to SEQ ID NO: 131
  REGION 1..123: note = chemically synthesized

SEQ ID NO: 171 - protein, 123 aa, synthetic construct; identical to SEQ ID NO: 132
  REGION 1..123: note = chemically synthesized

SEQ ID NO: 172 - protein, 121 aa, synthetic construct; identical to SEQ ID NO: 133
  REGION 1..121: note = chemically synthesized

SEQ ID NO: 173 - protein, 123 aa, synthetic construct; identical to SEQ ID NO: 134
  REGION 1..123: note = chemically synthesized

SEQ ID NO: 174 - protein, 121 aa, synthetic construct; identical to SEQ ID NO: 135
  REGION 1..121: note = chemically synthesized

SEQ ID NO: 175 - protein, 116 aa, synthetic construct
  REGION 1..116: note = chemically synthesized
MDTRAPTQLLGLLLLWLPGARCALVMTQTPSSTSTAVGGTVTIKCQASQSISVYLAWYQQKPGQPPKLLIYSASTLASGVPSRFKGSRSGTEYTLTISGVQREDAATYYCLGSAGS

SEQ ID NO: 176 - protein, 107 aa, synthetic construct; identical to SEQ ID NO: 137
  REGION 1..107: note = chemically synthesized

SEQ ID NO: 177 - protein, 106 aa, synthetic construct; identical to SEQ ID NO: 138
  REGION 1..106: note = chemically synthesized

SEQ ID NO: 178 - protein, 107 aa, synthetic construct; identical to SEQ ID NO: 139
  REGION 1..107: note = chemically synthesized

SEQ ID NO: 179 - protein, 108 aa, synthetic construct; identical to SEQ ID NO: 140
  REGION 1..108: note = chemically synthesized

SEQ ID NO: 180 - protein, 106 aa, synthetic construct; identical to SEQ ID NO: 141
  REGION 1..106: note = chemically synthesized

SEQ ID NO: 181 - protein, 106 aa, synthetic construct; identical to SEQ ID NO: 142
  REGION 1..106: note = chemically synthesized

SEQ ID NO: 182 - protein, 107 aa, synthetic construct; identical to SEQ ID NO: 143
  REGION 1..107: note = chemically synthesized

SEQ ID NO: 183 - protein, 120 aa, synthetic construct
  REGION 1..120: note = chemically synthesized
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 184 - protein, 110 aa, synthetic construct
  REGION 1..110: note = chemically synthesized
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 185 - protein, 118 aa, synthetic construct
  REGION 1..118: note = chemically synthesized
EVKLQESGPSLVKPSQTLSLTCSVTGYSITSDYWNWIRKFPGNKLEYVGYISYTGSTYYNPSLKSRISITRDTSKNQYYLQLNSVTSEDTATYYCARYGGWLSPFDYWGQGTTLTVSS

SEQ ID NO: 186 - protein, 120 aa, synthetic construct
  REGION 1..120: note = chemically synthesized
EVQLQESGPGLVAPSQSLSITCTVSGFSLTTYSINWIRQPPGKGLEWLGVMWAGGGTNSNSVLKSRLIISKDNSKSQVFLKMNSLQTDDTARYYCARYYGNSPYYAIDYWGQGTSVTVSS

SEQ ID NO: 187 - protein, 118 aa, synthetic construct
  REGION 1..118: note = chemically synthesized
EVKLQESGPSLVKPSQTLSLTCSVTGYSIISDYWNWIRKFPGNKLEYLGYISYTGSTYYNPSLKSRISITRDTSKNQYYLQLNSVTTEDTATYYCARRGGWLLPFDYWGQGTTLTVSS

SEQ ID NO: 188 - protein, 118 aa, synthetic construct
  REGION 1..118: note = chemically synthesized
EVKLQESGPSLVKPGASVKLSCKASGYTFTSYDINWVKQRPGQGLEWIGWIFPRDNNTKYNENFKGKATLTVDTSSTTAYMELHSLTSEDSAVYFCTKENWVGDFDYWGQGTTLTLSS

SEQ ID NO: 189 - protein, 114 aa, synthetic construct
  REGION 1..114: note = chemically synthesized
EVQLQQSGPDLVTPGASVRISCQASGYTFPDYYMNWVKQSHGKSLEWIGDIDPNYGGTTYNQKFKGKAILTVDRSSSTAYMELRSLTSEDSAVYYCARGALTDWGQGTSLTVSS

SEQ ID NO: 190 - protein, 106 aa, synthetic construct
  REGION 1..106: note = chemically synthesized
EIVLTQSPATLSLSPGERATLSCRASSSVSYIYWFQQKPGQSPRPLIYAAFNRATGIPARFSGSGSGTDYTLTISSLEPEDFAVYYCQQWSNNPLTFGQGTKVEIK

SEQ ID NO: 191 - protein, 114 aa, synthetic construct
  REGION 1..114: note = chemically synthesized
QVQLVQSGAEVKKPGASVKVSCKASGYTFPDYYMNWVRQAPGQGLEWMGDIDPNYGGTNYAQKFQGRVTMTRDTSISTAYMELSRLRSDDTAVYYCARGALTDWGQGTMVTVSS

SEQ ID NO: 192 - protein, 114 aa, synthetic construct
  REGION 1..114: note = chemically synthesized
QVQLVQSGAEVKKPGASVKVSCKASGYTFPDYYMNWVRQAPGQSLEWMGDIDPNYGGTNYNQKFQGRVTMTRDTSISTAYMELSRLRSDDTAVYYCARGALTDWGQGTMVTVSS

SEQ ID NO: 193 - protein, 114 aa, synthetic construct
  REGION 1..114: note = chemically synthesized
EVQLVQSGAEVKKPGASVKVSCKASGYTFPDYYMNWVRQAPGQSLEWMGDIDPNYGGTNYNQKFQGRVTMTVDRSSSTAYMELSRLRSDDTAVYYCARGALTDWGQGTMVTVSS

SEQ ID NO: 194 - protein, 114 aa, synthetic construct
  REGION 1..114: note = chemically synthesized
EVQLVESGGGLVQPGRSLRLSCTASGYTFPDYYMNWVRQAPGKGLEWVGDIDPNYGGTTYAASVKGRFTISVDRSKSIAYLQMSSLKTEDTAVYYCTRGALTDWGQGTMVTVSS

SEQ ID NO: 195 - protein, 114 aa, synthetic construct
  REGION 1..114: note = chemically synthesized
EVQLVESGGGLVQPGRSLRLSCTASGYTFPDYYMNWVRQAPGKGLEWVGDIDPNYGGTTYNASVKGRFTISVDRSKSIAYLQMSSLKTEDTAVYYCARGALTDWGQGTMVTVSS

SEQ ID NO: 196 - protein, 107 aa, synthetic construct
  REGION 1..107: note = chemically synthesized
DIVMTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 197 - protein, 107 aa, synthetic construct
  REGION 1..107: note = chemically synthesized
DIVTTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 198 - protein, 113 aa, synthetic construct
  REGION 1..113: note = chemically synthesized
DIVMTQSPSSLAVSVGEKVSMGCKSSQSLLYSSNQKNSLAWYQQKPGQSPKLLIDWASTRESGVPDRFTGSGSGTDFTLTISSVKAEDLAVYYCQQYYGYPLTFGAGTKLELK

SEQ ID NO: 199 - protein, 106 aa, synthetic construct
  REGION 1..106: note = chemically synthesized
DIVMTQSPAIMSASPGEKVTMTCSASSSIRYMHWYQQKPGTSPKRWISDTSKLTSGVPARFSGSGSGTSYALTISSMEAEDAATYYCHQRSSYPWTFGGGTKLEIK

SEQ ID NO: 200 - protein, 106 aa, synthetic construct
  REGION 1..106: note = chemically synthesized
QIVLSQSPAILSASPGEKVTMTCRASSSVSYIYWFQQKPGSSPKPWIYATFNLASGVPARFSGSGSGTSYSLTISRVETEDAATYYCQQWSNNPLTFGAGTKLELK

SEQ ID NO: 201 - protein, 106 aa, synthetic construct
  REGION 1..106: note = chemically synthesized
EIVLTQSPATLSLSPGERATLSCRASSSVSYIYWFQQKPGQAPRLLIYAAFNRATGIPARFSGSGSGTDYTLTISSLEPEDFAVYYCQQWSNNPLTFGQGTKVEIK

SEQ ID NO: 202 - protein, 106 aa, synthetic construct
  REGION 1..106: note = chemically synthesized
QIVLTQSPATLSLSPGERATLSCRASSSVSYIYWFQQKPGQSPRPLIYATFNLASGIPARFSGSGSGTSYTLTISRLEPEDFAVYYCQQWSNNPLTFGQGTKVEIK

SEQ ID NO: 203 - protein, 106 aa, synthetic construct
  REGION 1..106: note = chemically synthesized
DIQLTQSPSSLSASVGDRVTITCRASSGVSYIYWFQQKPGKAPKLLIYAAFNLASGVPSRFSGSGSGTEYTLTISSLQPEDFATYYCQQWSNNPLTFGQGTKVEIK

SEQ ID NO: 204 - protein, 106 aa, synthetic construct
  REGION 1..106: note = chemically synthesized
DIQLTQSPSSLSASVGDRVTITCRASSGVSYIYWFQQKPGKAPKPLIYAAFNLASGVPSRFSGSGSGTEYTLTISSLQPEDFATYYCQQWSNNPLTFGQGTKVEIK

SEQ ID NO: 205 - protein, 106 aa, synthetic construct
  REGION 1..106: note = chemically synthesized
DIQLTQSPSILSASVGDRVTITCRASSSVSYIYWFQQKPGKAPKPLIYATFNLASGVPSRFSGSGSGTSYTLTISSLQPEDFATYYCQQWSNNPLTFGQGTKVEIK

SEQ ID NO: 206 - protein, 124 aa, synthetic construct
  REGION 1..124: note = chemically synthesized
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARALPSGTILVGGWFDPWGQGTLVTVSS

SEQ ID NO: 207 - protein, 124 aa, synthetic construct
  REGION 1..124: note = chemically synthesized
EVQLVQSGGGVVQPGRSLRLSCAASGFTFSSYALSWVRQAPGKGLEWVSAISGGGGSTYYADSVKGRFTISRDNSKNTLYLQMNSLRAEDTAVYYCAKDVFPETFSMNYGMDVWGQGTLVTVSS

SEQ ID NO: 208 - protein, 128 aa, synthetic construct
  REGION 1..128: note = chemically synthesized
QVQLVQSGGGVVQPGGSLRLSCAASGFTFDDYAMHWVRQAPGKGLEWVSLISGDGGSTYYADSVKGRFTISRDNSKNSLYLQMNSLRTEDTALYYCAKVLLPCSSTSCYGSVGAFDIWGQGTTVTVSS

SEQ ID NO: 209 - protein, 117 aa, synthetic construct
  REGION 1..117: note = chemically synthesized
QVQLVQSGGSVVRPGESLRLSCVASGFIFDNYDMSWVRQVPGKGLEWVSRVNWNGGSTTYADAVKGRFTISRDNTKNSLYLQMNNLRAEDTAVYYCVREFVGAYDLWGQGTTVTVSS

SEQ ID NO: 210 - protein, 121 aa, synthetic construct
  REGION 1..121: note = chemically synthesized
QVQLVQSGAEVKKPGATVKVSCKVFGDTFRGLYIHWVRQAPGQGLEWMGGIIPIFGTANYAQKFQGRVTITTDESTSTAYMELSSLRSEDTAVYYCASGLRWGIWGWFDPWGQGTLVTVSS

SEQ ID NO: 211 - protein, 121 aa, synthetic construct
  REGION 1..121: note = chemically synthesized
EVQLVQSGAELKKPGSSVKVSCKAFGGTFSDNAISWVRQAPGQGPEWMGGIIPIFGKPNYAQKFQGRVTITADESTSTAYMVLSSLRSEDTAVYYCARTMVRGFLGVMDVWGQGTTVTVSS

SEQ ID NO: 212 - protein, 125 aa, synthetic construct
  REGION 1..125: note = chemically synthesized
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDQFVTIFGVPRYGMDVWGQGTTVTVSS

SEQ ID NO: 213 - protein, 120 aa, synthetic construct
  REGION 1..120: note = chemically synthesized
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRQMFGAGIDFWGPGTLVTVSS

SEQ ID NO: 214 - protein, 118 aa, synthetic construct
  REGION 1..118: note = chemically synthesized
EVQLVESGAEVKKPGSSVKVSCKVSGGTFGTYALNWVRQAPGQGLEWMGRIVPLIGLVNYAHNFEGRISITADKSTGTAYMELSNLRSDDTAVYYCAREVYGGNSDYWGQGTLVTVSS

SEQ ID NO: 215 - protein, 120 aa, synthetic construct
  REGION 1..120: note = chemically synthesized
QVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSS

SEQ ID NO: 216 - protein, 121 aa, synthetic construct
  REGION 1..121: note = chemically synthesized
QVQLQESGGGVVQPGRSLRLSCSASGFTFSRHGMHWVRQAPGKGLEWVAVISHDGSVKYYADSMKGRFSISRDNSNNTLYLQMDSLRADDTAVYYCARGLSYQVSGWFDPWGQGTLVTVSS

SEQ ID NO: 217 - protein, 110 aa, synthetic construct
  REGION 1..110: note = chemically synthesized
NFMLTQPHSVSESPGKTVTISCTRSSGSIASNYVQWYQQRPGSSPTTVIYEDNQRPSGVPDRFSGSIDTSSNSASLTISGLKTKDEADYYCQSYDGITVIFGGGTKLTVL

SEQ ID NO: 218 - protein, 111 aa, synthetic construct
  REGION 1..111: note = chemically synthesized
NFMLTQPHSVSGSPGKTVTLPCTRSSGSIASHYVQWYQQRPGSAPTTVIYEDNKRPSGVPDRFSGSIDSSSNSASLSISGLKTEDEADYYCQSYDSSNRWVFGGGTKLTVL

SEQ ID NO: 219 - protein, 115 aa, synthetic construct
  REGION 1..115: note = chemically synthesized
LPVLTQPASLSASPGASASLTCTLRSGLNVGSYRIYWYQQKPGSRPQYLLNYKSDSNKQQASGVPSRFSGSKDASANAGILLISGLQSEDEADYYCMIWYSSAVVFGGGTKLTVL

SEQ ID NO: 220 - protein, 111 aa, synthetic construct
  REGION 1..111: note = chemically synthesized
NFMLTQPHSVSESPGKTVTISCTRSSGNIASNYVQWYQQRPGSAPTTVIYEDNQRPSGVPDRFSGSIDSSSNSASLTISGLKTEDEADYYCQSYDSSNLWVFGGGTKLTVL

SEQ ID NO: 221 - protein, 108 aa, synthetic construct
  REGION 1..108: note = chemically synthesized
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHYVFGTGTKVTVL

SEQ ID NO: 222 - protein, 108 aa, synthetic construct
  REGION 1..108: note = chemically synthesized
LPVLTQAPSVSVAPGKTARITCGGSDIGRKSVHWYQQKPGQAPALVIYSDRDRPSGISERFSGSNSGNTATLTISRVEAGDEADYYCQVWDNNSDHYVFGAGTELIVL

SEQ ID NO: 223 - protein, 109 aa, synthetic construct
  REGION 1..109: note = chemically synthesized
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSTLPFGGGTKLTVL

SEQ ID NO: 224 - protein, 107 aa, synthetic construct
  REGION 1..107: note = chemically synthesized
EIVLTQSPATLSLSPGERATLSCRASQSIGNSLAWYQQKPGQAPRLLMYGASSRATGIPDRFSGSGAGTDFTLTISSLEPEDFATYYCQQHTIPTFSFGPGTKVEVK

SEQ ID NO: 225 - protein, 107 aa, synthetic construct
  REGION 1..107: note = chemically synthesized
DIVMTQTPSFLSASIGDRVTITCRASQGIGSYLAWYQQRPGEAPKLLIYAASTLQSGVPSRFSGSGSGTDFTLTISNLQPEDFATYYCQQLNNYPITFGQGTRLEIK

SEQ ID NO: 226 - protein, 105 aa, synthetic construct
  REGION 1..105: note = chemically synthesized
QSALTQPPSVSVSPGQTANIPCSGDKLGNKYAYWYQQKPGQSPVLLIYQDIKRPSRIPERFSGSNSADTATLTISGTQAMDEADYYCQTWDNSVVFGGGTKLTVL

SEQ ID NO: 227 - protein, 112 aa, synthetic construct
  REGION 1..112: note = chemically synthesized
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL

SEQ ID NO: 228 - protein, 110 aa, synthetic construct
  REGION 1..110: note = chemically synthesized
NFMLTQPHSVSESPGKTVTISCTRSSGNIGTNYVQWYQQRPGSAPVALIYEDYRRPSGVPDRFSGSIDSSSNSASLIISGLKPEDEADYYCQSYHSSGWEFGGGTKLTVL

SEQ ID NO: 229 - protein, 108 aa, synthetic construct
  REGION 1..108: note = chemically synthesized
QSVLTQPPSVSVAPGQTARITCGGNNIGSKGVHWYQQKPGQAPVLVVYDDSDRPSGIPERFSGSNSGNTATLTISRVEAGDEADYYCQVWDSSSDHWVFGGGTKLTVL

SEQ ID NO: 230 - protein, 111 aa, synthetic construct
  REGION 1..111: note = chemically synthesized
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSTTPSVFGGGTKLTVL

SEQ ID NO: 231 - protein, 120 aa, synthetic construct
  REGION 1..120: note = chemically synthesized
QVQLVQSGAEVKKPGASVKVSCKASGYTFTSYGISWVRQAPGQGLEWMGWTSPHNGLTAFAQILEGRVTMTTDTSTNTAYMELRNLTFDDTAVYFCAKVHPVFSYALDVWGQGTLVTVSS

SEQ ID NO: 232 - protein, 118 aa, synthetic construct
  REGION 1..118: note = chemically synthesized
EVQLVESGAEVMNPGSSVRVSCRGSGGDFSTYAFSWVRQAPGQGLEWMGRIIPILGIANYAQKFQGRVTITADKSTSTAYMELSSLRSDDTAVYYCARDGYGSDPVLWGQGTLVTVSS

SEQ ID NO: 233 - protein, 118 aa, synthetic construct
  REGION 1..118: note = chemically synthesized
EVQLVQSGAEVKKPGASVKVSCKASGYTFTNYGISWVRQAPGQGLEWMGWISAYNGNTNYAQKVQGRVTMTTDTSTSTGYMELRSLRSDDTAVYYCARGDFRKPFDYWGQGTLVTVSS

SEQ ID NO: 234 - protein, 115 aa, synthetic construct
  REGION 1..115: note = chemically synthesized
EVQLVQSGPELKKPGASVKMSCKASGYTFTSYVMHWVKQAPGQRLEWIGYVNPFNDGTKYNEMFKGRATLTSDKSTSTAYMELSSLRSEDSAVYYCARQAWGYPWGQGTLVTVSS

SEQ ID NO: 235 - protein, 115 aa, synthetic construct
  REGION 1..115: note = chemically synthesized
EVQLVQSGAEVKKPGASVKMSCKASGYTFTSYVMHWVKQAPGQRLEWIGYVNPFNDGTKYNEMFKGRATLTSDKSTSTAYMELSSLRSEDTAVYYCARQAWGYPWGQGTLVTVSS

SEQ ID NO: 236 - protein, 115 aa, synthetic construct
  REGION 1..115: note = chemically synthesized
EVQLVQSGAEVKKPGASVKMSCKASGYTFTSYVMHWVRQAPGQRLEWIGYVNPFNDGTKYNEMFKGRATLTSDKSTSTAYMELSSLRSEDTAVYYCARQAWGYPWGQGTLVTVSS

SEQ ID NO: 237 - protein, 115 aa, synthetic construct
  REGION 1..115: note = chemically synthesized
EVQLVQSGAEVKKPGASVKVSCKASGYTFTSYVMHWVRQAPGQRLEWIGYVNPFNDGTKYNEMFKGRATLTSDKSTSTAYMELSSLRSEDTAVYYCARQAWGYPWGQGTLVTVSS

SEQ ID NO: 238 - protein, 115 aa, synthetic construct
  REGION 1..115: note = chemically synthesized
EVQLVQSGAEVKKPGASVKVSCKASGYTFTSYVMHWVRQAPGQRLEWIGYVNPFNDGTKYNEMFKGRATITSDKSTSTAYMELSSLRSEDTAVYYCARQAWGYPWGQGTLVTVSS

SEQ ID NO: 239 - protein, 111 aa, synthetic construct
  REGION 1..111: note = chemically synthesized
DIVLTQSPASLALSPGERATLSCRATESVEYYGTSLVQWYQQKPGQPPKLLIYAASSVDSGVPSRFSGSGSGTDFTLTINSLEEEDAAMYFCQQSRRVPYTFGQGTKLEIK

SEQ ID NO: 240 - protein, 111 aa, synthetic construct
  REGION 1..111: note = chemically synthesized
DIVLTQSPATLSLSPGERATLSCRATESVEYYGTSLVQWYQQKPGQPPKLLIYAASSVDSGVPSRFSGSGSGTDFTLTINSLEAEDAAMYFCQQSRRVPYTFGQGTKLEIK

SEQ ID NO: 241 - protein, 111 aa, synthetic construct
  REGION 1..111: note = chemically synthesized
EIVLTQSPATLSLSPGERATLSCRATESVEYYGTSLVQWYQQKPGQPPKLLIYAASSVDSGVPSRFSGSGSGTDFTLTINSLEAEDAAMYFCQQSRRVPYTFGQGTKLEIK

SEQ ID NO: 242 - protein, 111 aa, synthetic construct
  REGION 1..111: note = chemically synthesized
DIVLTQSPATLSLSPGERATLSCRATESVEYYGTSLVQWYQQKPGQPPKLLIYAASSVDSGVPSRFSGSGSGTDFTLTINSLEAEDAATYFCQQSRRVPYTFGQGTKLEIK

SEQ ID NO: 243 - protein, 123 aa, synthetic construct
  REGION 1..123: note = chemically synthesized
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREGTIYDSSGYSFDYWGQGTLVTVSS

SEQ ID NO: 244 - protein, 124 aa, synthetic construct
  REGION 1..124: note = chemically synthesized
EVQLVQSGAEVKKPGSSVKVSCKASGGTFSSYAISWVRQAPGQGLEWMGIINPSGGSTSYAQKFQGRVSMTRDTSTSTVYMELSSLTSEDTAVYYCARDLFPHIYGNYYGMDIWGQGTTVTVSS

SEQ ID NO: 245 - protein, 118 aa, synthetic construct
  REGION 1..118: note = chemically synthesized
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLAVPGAFDIWGQGTMVTVSS

SEQ ID NO: 246 - protein, 116 aa, synthetic construct
  REGION 1..116: note = chemically synthesized
EVQLVESGGGVVQPGRSLRLSCAASGFTFSSYAMHWVRQAPGKGLAVISYDGSNKYYADSVKGRFTISRDNSKNTLYLQMNSLRAEDTAVYYCARGQWLVTELDYWGQGTLVTVSS

SEQ ID NO: 247 - protein, 122 aa, synthetic construct
  REGION 1..122: note = chemically synthesized
EVQLVESGSEVEKPGSSVKVSCKASGGTFSDSGISWVRQAPGQGLEWMGGIIPMFATPYYAQKFQDRVTITADESTSTVYMELSGLRSDDTAVFYCARDRGRGHLPWYFDLWGRGTLVTVSS

SEQ ID NO: 248 - protein, 119 aa, synthetic construct
  REGION 1..119: note = chemically synthesized
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 249 - protein, 124 aa, synthetic construct
  REGION 1..124: note = chemically synthesized
EVQLLESGAEVKKPGSSVKVSCKASGGTLSRYALSWVRQAPGQGPEWVGAIIPIFGTPHYSKKFQDRVIITVDTSTNTAFMELSSLRFEDTALYFCARGHDEYDISGYHRLDYWGQGTLVTVSS

SEQ ID NO: 250 - protein, 121 aa, synthetic construct
  REGION 1..121: note = chemically synthesized
QVQLVQSGSELKKPGSSVKVSCKASGYSFSGYYIHWVRQAPGQGLEWMGWIDPNSGVTNYVRRFQGRVTMTRDTSLSTAYMELSGLTADDTAVYYCARDENLWQFGYLDYWGQGTLVTVSS

SEQ ID NO: 251 - protein, 120 aa, synthetic construct
  REGION 1..120: note = chemically synthesized
QVQLVQSGAEVKKPGSSVKVSCKASGGTFSRYGVHWVRQAPGQGLEWMGRLIPIVSMTNYAQKFQDRVSITTDKSTGTAYMELRSLTSEDTALYYCASVGQQLPWVFFAWGQGTLVTVSS

SEQ ID NO: 252 - protein, 118 aa, synthetic construct
  REGION 1..118: note = chemically synthesized
QVQLVESGGGVVQPGRSLRLSCAASGFTFSSYAMHWVRQAPGKGLEWVAVISFDGSNKYYADSVRGRFTISRDNSKNTLYLQMNSLRTEDTAVYYCARGWLDRDIDYWGQGTLVTVSS

SEQ ID NO: 253 - protein, 118 aa, synthetic construct
  REGION 1..118: note = chemically synthesized
EVQLVESGGGVVQPGRSLRLSCAASGFTFSSYAMHWVRQAPGKGLEWVAVISFDGSNKYYADSVRGRFTISRDNSKNTLYLQMNSLRTEDTAVYYCARGWLDRDIDYWGQGTLVTVSS

SEQ ID NO: 254 - protein, 121 aa, synthetic construct
  REGION 1..121: note = chemically synthesized
EVQLVQSGGGLVQPGGSLRLSCAASGFTFSDYGMHWVRQPPGKGLEWLAVISYDGSYKIHADSVQGRFTISRDNAKNSVFLQMNSLKTEDTAVYYCTTDRKWLAWHGMDVWGQGTTVTVSS

SEQ ID NO: 255 - protein, 118 aa, synthetic construct
  REGION 1..118: note = chemically synthesized
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGIVADFQHWGQGTLVTVSS

SEQ ID NO: 256 - protein, 121 aa, synthetic construct
  REGION 1..121: note = chemically synthesized
EVQLVESGAEVKKPGASVKVSCKASGDTFSRYGITWVRQAPGRGLEWMGNIVPFFGATNYAQKFQGRLTITADKSSYTSYMDLSSLRSDDTAVYYCARDHFYGSGGYFDYWGQGTLVTVSS

SEQ ID NO: 257 - protein, 121 aa, synthetic construct
  REGION 1..121: note = chemically synthesized
EVQLLESGAEVKKPGASVKVSCKASGYTFNSYDINWVRQAPGQGLEWMGGIIPVFGTANYAESFQGRVTMTADHSTSTAYMELNNLRSEDTAVYYCARDRWHYESRPMDVWGQGTTVTVSS

SEQ ID NO: 258 - protein, 119 aa, synthetic construct
  REGION 1..119: note = chemically synthesized
EVQLVESGGGLVRPGGSLRLACAASGFSFSDYYMTWIRQAPGRGLEWIAYISDSGQTVHYADSVKGRFTISRDNTKNSLFLQVNTLRAEDTAVYYCAREDLLGYYLQSWGQGTLVTVSS

SEQ ID NO: 259 - protein, 131 aa, synthetic construct
  REGION 1..131: note = chemically synthesized
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDEPRAVAGSQAYYYYGMDVWGQGTTVTVSS

SEQ ID NO: 260 - protein, 124 aa, synthetic construct
  REGION 1..124: note = chemically synthesized
EVQLVQSGAEVKKPGASVKVSCKASGYTFTSYYMHWVRQAPGQGLEWMGIINPSDGSTSYAQKFQGRVTMTRDTSTSTVHMELSSLRSEDTAVYYCARDLFPHIYGNYYGMDIWGQGTTVTVSS

SEQ ID NO: 261 - protein, 118 aa, synthetic construct
  REGION 1..118: note = chemically synthesized
QMQLVQSGGGVVQPGRSLRLSCAASGFTFSSYAMHWVRQAPGKGLEWVAVISFDGSNKYYADSVRGRFTISRDNSKNTLYLQMNSLRTEDTAVYYCARGWLDRDIDYWGQGTLVTVSS

SEQ ID NO: 262 - protein, 118 aa, synthetic construct
  REGION 1..118: note = chemically synthesized
QVQLVQSGGGVVQPGRSLRLSCAASGFTFSSYAMHWVRQAPGKGLEWVAVISFDGSNKYYADSVRGRFTISRDNSKNTLYLQMNSLRTEDTAVYYCARGWLDRDIDYWGQGTLVTVSS

SEQ ID NO: 263 - protein, 110 aa, synthetic construct
  REGION 1..110: note = chemically synthesized
QSVLTQPPSVSAAPGQKVTISCSGNNSNIANNYVSWYQQLPGTAPKLLIYDNNYRPSGIPDRFSGSKSGTSATLDITGLQTGDEADYYCGVWDGSLTTGVFGGGTKLTVL

SEQ ID NO: 264 - protein, 107 aa, synthetic construct
  REGION 1..107: note = chemically synthesized
AIQMTQSPSSLSASVGDRVTITCRASQGISNYLAWYQQKPGKVPKLLIYAASTLESGVPSRFSGSGSGTDFTLTISSLQPEDLATYYCQQLHTFPLTFGGGTKVEIK

SEQ ID NO: 265 - protein, 111 aa, synthetic construct
  REGION 1..111: note = chemically synthesized
QPVLTQPPSASGSPGQSVTISCTGTSSDVGAYNFVSWYRQHPGKAPKLMIYEVNKRPSGVPDRFSGSKSGNTASLTVSGLQAEDEADYYCSSYAGTNSLGIFGTGTKLTVL

SEQ ID NO: 266 - protein, 111 aa, synthetic construct
  REGION 1..111: note = chemically synthesized
QSVVTQPPSVSAAPGQKVTISCSGSSSDIGNHYVSWYQQLPGTAPKLLIYDNNQRPSGIPDRFSGSKSGTSATLAITGLQTGDEADYYCGTWDNSLSPHLLFGGGTKLTVL

SEQ ID NO: 267 - protein, 110 aa, synthetic construct
  REGION 1..110: note = chemically synthesized
QSVLTQPPSVSAAPGQKVTISCSGSSSNMGNNYVSWYKQVPGTAPKLLIYENDKRPSGIPDRFSGSKSGTSATLGITGLQTGDEADYYCGTWDNSLSGFVFASGTKVTVL

SEQ ID NO: 268 - protein, 109 aa, synthetic construct
  REGION 1..109: note = chemically synthesized
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 269 - protein, 110 aa, synthetic construct
  REGION 1..110: note = chemically synthesized
QSVLTQPASVSGSPGQSITISCTGTSSDVGSYNLVSWYQQHPGKAPKLMIYEVSKRPSGVSNRFSGSKSGNTASLTISGLQAEDEADYYCSSYGGFNNLLFGGGTKLTVL

SEQ ID NO: 270 - protein, 107 aa, synthetic construct
  REGION 1..107: note = chemically synthesized
DIVMTQSPSSLSASIGDRVTITCRASQRISAYVNWYQQKPGKAPKVLIYAASSLRSGVPSRFSGSGSGTDFTLTISSLQPEDFATYYCQQTYSSPWTFGQGTKVEIK

SEQ ID NO: 271 - protein, 112 aa, synthetic construct
  REGION 1..112: note = chemically synthesized
QSVLTQPPSASGSPGQSVTISCTGTSSDIGGYDSVSWYQQHPGKAPKLMIYDVSKRPSGVSNRFSGSKSGNTASLTISGLQAEDEADYYCSSYTSSSIFFYVFGTGTKVTVL

SEQ ID NO: 272 - protein, 110 aa, synthetic construct
  REGION 1..110: note = chemically synthesized
LPVLTQPASVSGSPGQSITISCTGTTSDIGGYDYVSWYQQHPGKAPKLMIYDVSKRPSGVSNRFSGSKSGNTASLTISGLQAEDEADYYCSSYTSSSTHVFGTGTKLTVL

SEQ ID NO: 273 - protein, 111 aa, synthetic construct
  REGION 1..111: note = chemically synthesized
QSALTQPASVSGSPGQSITISCTGTSSDVGGYNYVSWYQQHPGKAPKLMIYDVSNRPSGVSNRFSGSKSGNTASLTISGLQAEDEADYYCSSYRSSTLGPVFGGGTKLTVL

SEQ ID NO: 274 - protein, 110 aa, synthetic construct
  REGION 1..110: note = chemically synthesized
QAGLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNGYVFGTGTKLTVL

SEQ ID NO: 275 - protein, 110 aa, synthetic construct
  REGION 1..110: note = chemically synthesized
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSTTHVFGTGTKVTVL

SEQ ID NO: 276 - protein, 110 aa, synthetic construct
  REGION 1..110: note = chemically synthesized
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVWVFGGGTQLTVL

SEQ ID NO: 277 - protein, 112 aa, synthetic construct
  REGION 1..112: note = chemically synthesized
QSVLTQPASVSGSPGQSITISCTGTSSDVGGYNYVSWYQQHPGRAPRLMIYDVSNRPSGVSNRFSGSKSGNTASLTISGLQAEDEGDYYCSSYTSGGTLGPVFGGGTKLTVL

SEQ ID NO: 278 - protein, 110 aa, synthetic construct
  REGION 1..110: note = chemically synthesized
QAGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 279 - protein, 107 aa, synthetic construct
  REGION 1..107: note = chemically synthesized
AIRMTQSPSSLSASVGDRVTITCRASQSISNYLNWYQQRPGKAPNLLIYAASSLQSGVPSRFSGSGSGTDFTLTISSLQPEDFATYYCQQTYSTPYTFGQGTKLEIK

SEQ ID NO: 280 - protein, 111 aa, synthetic construct
  REGION 1..111: note = chemically synthesized
QSVLTQPASVSGSPGQSITISCTGTSSDVGGYNYVSWYRQHPGKAPKLMIYDVSYRPSGVSNRFSGSKSGNTASLTISGLQAEDEADYYCSSYTDSSTRYVFGTGTKLTVL

SEQ ID NO: 281 - protein, 110 aa, synthetic construct
  REGION 1..110: note = chemically synthesized
QPVLTQPPSASGTPGQRVAISCSGSRSNIEINSVNWYQQLPGTAPKLLIYDNNKRPSGIPDRFSGSKSGTSATLGITGLQTGDEADYYCGSWDSSLSADVFGTGTKLTVL

SEQ ID NO: 282 - protein, 110 aa, synthetic construct
  REGION 1..110: note = chemically synthesized
QSVLTQPPSVSAAPGKKVTISCSGSSSNIGNNYVSWYQQLPGTAPKLLIYRNNQRPSGVPDRFSGSKSGTSASLAISGLQSEDEADYYCATWDDSLNGWVFGGGTKLTVL

SEQ ID NO: 283 - protein, 111 aa, synthetic construct
  REGION 1..111: note = chemically synthesized
QSVVTQPPSVSGAPGQRVTISCTGSSSNIGAGYDVHWYQQLPGTAPKLLIYGNNNRHSGVPDRFSGSKSGTSASLAITGLQAEDEAEFFCGTWDSRLTTYVFGSGTKLTVL

SEQ ID NO: 284 - protein, 110 aa, synthetic construct
  REGION 1..110: note = chemically synthesized
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 285 - protein, 103 aa, synthetic construct
  REGION 1..103: note = chemically synthesized
VIWMTQSPSSLSASVGDRVTITCAASSLQSWYQQKPGKAPKLLIYEASTLESGVPSRFSGSGSGTEFTLTISSLQPEDFATYYCQQSYSTPYTFGQGTKLEIK

SEQ ID NO: 286 - protein, 110 aa, synthetic construct
  REGION 1..110: note = chemically synthesized
QSVVTQPPSVSAAPGQKVTISCSGSSSNIGNNYVSWYQQVPGTAPKLLIYDNNKRPSGIPDRFSGSNSDTSATLGITGLQTGDEADYYCGTWDSSLSAWVFGGGTKLTVL

SEQ ID NO: 287 - protein, 112 aa, synthetic construct
  REGION 1..112: note = chemically synthesized
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGSVVFGGGTKLTVL

SEQ ID NO: 288 - protein, 108 aa, synthetic construct
  REGION 1..108: note = chemically synthesized
SYELMQPPSVSVAPGKTATIACGGENIGRKTVHWYQQKPGQAPVLVIYYDSDRPSGIPERFSGSNSGNTATLTISRVEAGDEADYYCLVWDSSSDHRIFGGGTKLTVL

SEQ ID NO: 289 - protein, 108 aa, synthetic construct
  REGION 1..108: note = chemically synthesized
SYELMQPPSVSVAPGKTATIACGGENIGRKTVHWYQQKPGQAPVLVIYYDSDRPSGIPERFSGSNSGNTATLTISRVEAGDEADYYCQVWDSSSDHRIFGGGTKLTVL

SEQ ID NO: 290 - protein, 108 aa, synthetic construct; identical to SEQ ID NO: 289
  REGION 1..108: note = chemically synthesized

SEQ ID NO: 291 - protein, 108 aa, synthetic construct; identical to SEQ ID NO: 289
  REGION 1..108: note = chemically synthesized

SEQ ID NO: 292 - protein, 447 aa, synthetic construct
  REGION 1..447: note = chemically synthesized
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 440 aa, synthetic construct
  REGION 1..440: note = chemically synthesized
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 294 - protein, 218 aa, synthetic construct
  REGION 1..218: note = chemically synthesized
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 214 aa, synthetic construct
  REGION 1..214: note = chemically synthesized
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 122 aa, synthetic construct
  REGION 1..122: note = chemically synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 297 - protein, 118 aa, synthetic construct [sequence omitted]

SEQ ID NO: 298 - protein, 447 aa, synthetic construct
  REGION 1..447: note = chemically synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 299 - protein, 108 aa, synthetic construct; identical to SEQ ID NO: 149
  REGION 1..108: note = chemically synthesized

SEQ ID NO: 300 - protein, 214 aa, synthetic construct
  REGION 1..214: note = chemically synthesized
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 121 aa, synthetic construct
  REGION 1..121: note = chemically synthesized
EVQLVESGGGLVQPGGSLRLSCAASGFTFSRFWMSWVRQAPGKGLEWVANINQDGTEKYYVDSVKGRFTISRDNAKNSLYLQMNSLRAGDTAVYYCANTYYDFWSGHFDYWGQGTLVTVSS

SEQ ID NO: 302 - protein, 121 aa, synthetic construct
  REGION 1..121: note = chemically synthesized
QEHLVESGGGVVQPGRSLRLSCEASGFTFSNFGMHWVRQAPGKGLEWVAALWSDGSNKYYADSVKGRVTISRDNSKNTLYLQMNSLRAEDTAVYYCARGRGAPGIPIFGYWGQGTLVTVSS

SEQ ID NO: 303 - protein, 130 aa, synthetic construct
  REGION 1..130: note = chemically synthesized
EVQLVESGGGLVKPGGSLRLSCAASGFTFSNAWMSWVRQAPGKGLEWVGRIKRKTDGGTTDYAAPVKGRFTISRDDSKNTLHLQMNSLKTEDTAVYYCTTDDIVVVPAVMREYYFGMDVWGQGTTVTVSS

SEQ ID NO: 304 - protein, 121 aa, synthetic construct
  REGION 1..121: note = chemically synthesized
QVQLVQSGAEVKKPGASVQVSCKASGYSFTGYYIHWVRQAPGQGLEWMGWINPNSGTKKYAHKFQGRVTMTRDTSIDTAYMILSSLISDDTAVYYCARDEDWNFGSWFDSWGQGTLVTVSS

SEQ ID NO: 305 - protein, 121 aa, synthetic construct
  REGION 1..121: note = chemically synthesized
QVHLVQSGAEVKKPGASVKVSCKASGYTFTGYYIHWVRQAPGHGLEWMGWLNPNTGTTKYIQNFQGRVTMTRDTSSSTAYMELTRLRSDDTAVYYCARDEDWNYGSWFDTWGQGTLVTVSS

SEQ ID NO: 306 - protein, 120 aa, synthetic construct
  REGION 1..120: note = chemically synthesized
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 307 - protein, 120 aa, synthetic construct
  REGION 1..120: note = chemically synthesized
EVQLVESGGGVVRPGGSLRLSCAASGFTFDDYGMTWVRQVPGKGLEWVSGIHWSGRSTGYADSVKGRFTISRDNAKNSLYLQMNSLRAEDTALYYCARGGMSTGDWFDPWGQGTLVTVSS

SEQ ID NO: 308 - protein, 115 aa, synthetic construct
  REGION 1..115: note = chemically synthesized
EVQLVESGGGLVQPGGSLRLSCAASGFTVGSNYMNWVRQAPGKGLEWVSVIYSGGSTYYADSVKGRFTISRLTSKNTLYLQMSSLRPEDTAVYYCARGIRGLDVWGQGTTVTVSS

SEQ ID NO: 309 - protein, 115 aa, synthetic construct
  REGION 1..115: note = chemically synthesized
EERLVESGGDLVQPGGSLRLSCAASGITVGTNYMNWVRQAPGKGLEWVSVISSGGNTHYADSVKGRFIMSRQTSKNTLYLQMNSLETEDTAVYYCARGIRGLDVWGQGTMVTVSS

SEQ ID NO: 310 - protein, 118 aa, synthetic construct
  REGION 1..118: note = chemically synthesized
QVQLVQSGAEVKMPGSSVRVSCKASGGIFSSSTISWVRQAPGQGLEWMGEIIPVFGTVNYAQKFQDRVIFTADESTTTAYMELSSLKSGDTAVYFCARNWGLGSFYIWGQGTMVTVSS

SEQ ID NO: 311 - protein, 115 aa, synthetic construct
  REGION 1..115: note = chemically synthesized
EVQLVESGGDLVHPGRSLRLSCAASGFPFDEYAMHWVRQVPGKGLEWVSGISWSNNNIGYADSVKGRFTISRDNAKNSLYLQMNSLRPEDTAFYYCAKSGIFDSWGQGTLVTVSS

SEQ ID NO: 312 - protein, 118 aa, synthetic construct
  REGION 1..118: note = chemically synthesized
EVQLVESGGGVVQPGRSLRLSCAASGFTFSSYGMHWVRQAPGKGLEWVTLISYEGRNKYYADSVKGRFTISRDNSKNTLYLQMNSLRAEDTAVYYCAKDRTLYGMDVWGQGTTVTVSS

SEQ ID NO: 313 - protein, 121 aa, synthetic construct
  REGION 1..121: note = chemically synthesized
QVTLRESGPALVKTTQTLTLTCTFSGFSLSTNRMCVTWIRQPPGKALEWLARIDWDGVKYYNTSLKTRLTISKDTSKNQVVLTMTNMDPVDTATFYCARSTSLTFYYFDYWGQGTLVTVSS

SEQ ID NO: 314 - protein, 115 aa, synthetic construct
  REGION 1..115: note = chemically synthesized
EVQLVESGGGLVQPGGSLRLSCAASEFTVGTNHMNWVRQAPGKGLEWVSVIYSGGNTFYADSVKGRFTISRHTSKNTLYLQMNSLTAEDTAVYYCARGLGGMDVWGQGTTVTVSS

SEQ ID NO: 315 - protein, 120 aa, synthetic construct
  REGION 1..120: note = chemically synthesized
EVQLVESGGGLVQRGESLRLYCAASGFTFSKYWMNWVRQAPGKGLEWVANIKGDGSEKYYVDSVKGRFTISRDNAKNSLYLQMNSLRAEDTAVYYCARDYWGSGYYFDFWGQGTLVTVSS

SEQ ID NO: 316 - protein, 130 aa, synthetic construct
  REGION 1..130: note = chemically synthesized
EVQLVESGGGLVQSGGSLRLSCAASGFTFSSYWMSWVRQAPGKGLEWVANIKQDGSEKYYVDSVKGRFTISRDNAKNSLYLQMNSLRADDTAVYYCARDDIVVVPAPMGYYYYYFGMDVWGQGTTVTVSS

SEQ ID NO: 317 - protein, 123 aa, synthetic construct
  REGION 1..123: note = chemically synthesized
EVQLVESGGGLVQPGRSLRLSCAASGFTFDDFAMHWVRQAPGKGLEWVSGISWTGGNMDYANSVKGRFTISREDAKNSLYLQMNSLRAADTALYYCVKDIRGIVATGGAFDIWGRGTMVTVSS

SEQ ID NO: 318 - protein, 115 aa, synthetic construct
  REGION 1..115: note = chemically synthesized
EVQLVESGGGLVQPGGSLRLSCAASGFTVGTNYMNWVRQAPGKGLEWISVIYSGGSTFYADSVKGRFTISRQTSQNTLYLQMNSLRPEDTAVYYCARGIRGFDIWGQGTMVTVSS

SEQ ID NO: 319 - protein, 115 aa, synthetic construct
  REGION 1..115: note = chemically synthesized
EVQLVESGGGLVQPGGSLRLSCAASGFTISTNYMNWVRQAPGKGLEWVAVIYSSGSTYYIDSVKGRFTISRLTSKNTVYLQMSSLNSEDTAVYYCARGIRGFDIWGQGTMVTVSS

SEQ ID NO: 320 - protein, 124 aa, synthetic construct
  REGION 1..124: note = chemically synthesized
EVQLVESGGGLVQPGRSLRLSCAASGFTIDDSAMHWVRQTPGKGLEWVSGISWKSGSIGYADSVRGRFTISRDNAKNSLYLQMNSLRVEDTALYYCVKDIRGNWNYGGNWFDPWGQGTLVTVSS

SEQ ID NO: 321 - protein, 115 aa, synthetic construct
  REGION 1..115: note = chemically synthesized
EVQLVESGGGLVQPGGSLRLSCEASGFTVGVNHMNWVRQAPGKGLEWVSVIFSSGRTFYGDYVKGRLTIFRQTSQNTVYLQMNSLRSEDTAIYYCARGIGGLDIWGRGTMVTVSS

SEQ ID NO: 322 - protein, 123 aa, synthetic construct
  REGION 1..123: note = chemically synthesized
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSS

SEQ ID NO: 323 - protein, 120 aa, synthetic construct
  REGION 1..120: note = chemically synthesized
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSS

SEQ ID NO: 324 - protein, 114 aa, synthetic construct
  REGION 1..114: note = chemically synthesized
EVQLVESGGGLVQPGGSLRLSCAASGFTVGTNFMNWVRQAPGKGLEWVSAIYSGGTANYADSVKGRFTISRDTSRNTLYLQMNSLRTEDTAVYYCARGGGMDVWGQGTTVTVSS

SEQ ID NO: 325 - protein, 118 aa, synthetic construct
  REGION 1..118: note = chemically synthesized
QVQLVQSGAEVKKPGSSVKVSCKASGGTFNTYVLSWVRQAPGQGLEWMGEIIPILGAANYAQNFQGRVTFTTDESTNTAYMDLSSLRSEDTAVYYCARDRTSGGFDPWGQGTLVTVSS

SEQ ID NO: 326 - protein, 119 aa, synthetic construct
  REGION 1..119: note = chemically synthesized
QVQLVQSGAEVEKPGASVKVSCKASGYIFTHYGISWVRQAPGQGLEWVGWISPYNGYTDYAQKLQGRVTLTTDTSTTTAYMELRNLRSDDTAMYYCSRGRGPYWSFDLWGRGTLVTVSS

SEQ ID NO: 327 - protein, 106 aa, synthetic construct
  REGION 1..106: note = chemically synthesized
DIQMTQSPSTLSASVGDRVTITCRASQSISNWLAWYQQKPGKAPKLLIYKASSLESGVPSRFSGSGSGTEFTLTISSLQPDDFATYYCQQYHSYSYTFGQGTKEIK

SEQ ID NO: 328 - protein, 107 aa, synthetic construct
  REGION 1..107: note = chemically synthesized
DIQMTQSPSSLSASVGDRVTITCRASQGIRNDLGWYQQKPGKAPKRLIYTASSLQSGVPSRFSGSGSGTEFTLTISSLQPEDFATYYCLQHNSYPLTFGGGTKVAIK

SEQ ID NO: 329 - protein, 107 aa, synthetic construct
  REGION 1..107: note = chemically synthesized
DIQMTQSPSSLSASVGDRVTITCRTSQGIRNDLGWYQQKPGKAPKRLIYAASSLQSGVPSRFSGSGSGTEFTLTISSLQPEDFATYYCLQHNNYPYTFGQGTKLEIK

SEQ ID NO: 330 - protein, 112 aa, synthetic construct
  REGION 1..112: note = chemically synthesized
DIVMTQTPLSSPVTLGQPASISCRSSQTLVHGDGNTYLSWIQQRPGQPPRLLIYKVSNQFSGVPDRFSGSGAGTDFTLKISRVEAEDVGLYFCMQATHFPITFGQGTRLEIK

SEQ ID NO: 331 - protein, 112 aa, synthetic construct
  REGION 1..112: note = chemically synthesized
DIVMTQTPLSSPVTLGQPASISCRSSPSLVHSDGNTYLSWLQQRPGQPPRLLIYKISNRFSGVPDRFSGSGAGTDFTLKISRVEAEDVGVYYCMQATHFPITFGQGTRLEIR

SEQ ID NO: 332 - protein, 108 aa, synthetic construct
  REGION 1..108: note = chemically synthesized
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

SEQ ID NO: 333 - protein, 108 aa, synthetic construct
  REGION 1..108: note = chemically synthesized
DIQMTQSPSSLSASVGDRVTITCRASQSISSYLNWYQQKPGKAPKLLIYVASSLQSGVPSRFSGSGSGTDFTLTISSLQPEDFATYYCQQSYSTPPITFGQGTRLEIK

SEQ ID NO: 334 - protein, 108 aa, synthetic construct
  REGION 1..108: note = chemically synthesized
DIQMTQSPSSLSASVGDRVTITCRASQTINIYLNWYQQKPGRAPRLLIYAASSLQSGVPSRFSGSGSGTDFTLTISSLQPEDFATYYCHQSYSTPPITFGQGTRLEIK

SEQ ID NO: 335 - protein, 108 aa, synthetic construct
  REGION 1..108: note = chemically synthesized
DIQMTQSPSSLSASVGDRVTITCRASQSMSSYLNWYQQKPGRAPKLLIFAASSLQSGVPSRFSGSGSGTDFTLTISSLQPEDFATYYCQQSYSTPPITFGQGTRLEIK

SEQ ID NO: 336 - protein, 108 aa, synthetic construct
  REGION 1..108: note = chemically synthesized
EIVLTQSPGTLSLSPGERATLSCRASQSFNFNYLAWYQQKPGQAPRLLIYGASSRATGIPDRFSGSGSGTDFTLTINRLEPEDFGVFYCQQYESAPWTFGQGTKVEIK

SEQ ID NO: 337 - protein, 105 aa, synthetic construct
  REGION 1..105: note = chemically synthesized
DIQMTQSPSSLSASVGDRVTITCRASQSISSYLNWYQQKPGKLLIYAASSLQSGVPSRFSGGGSGTDFTLTISSLRPEDFATYYCQQSYCTPPITFGQGTRLEIK

SEQ ID NO: 338 - protein, 108 aa, synthetic construct
  REGION 1..108: note = chemically synthesized
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 339 - protein, 91 aa, synthetic construct
  REGION 1..91: note = chemically synthesized
DRVTITCRASQVISNYLAWYQQKPGKVPRLLIYAASTLQSGVPSRFSGSGSGTDFTLTISSLQPEDVATYYCQKYNSAPRTFGQGTKVEIK

SEQ ID NO: 340 - protein, 107 aa, synthetic construct
  REGION 1..107: note = chemically synthesized
DIQMTQSPSSLSASVGDRVTITCRASQNINNYLNWYQQKPGKAPKLLIYAASSFQNAVPSRFSGSGSGTDFTLTISSLQPEDFATYYCQQSYNTPLTFGGGTKVEIK

SEQ ID NO: 341 - protein, 107 aa, synthetic construct
  REGION 1..107: note = chemically synthesized
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 342 - protein, 108 aa, synthetic construct; identical to SEQ ID NO: 338
  REGION 1..108: note = chemically synthesized

SEQ ID NO: 343 - protein, 115 aa, synthetic construct
  REGION 1..115: note = chemically synthesized
QSLEESGGRLVKPDETLTITCTVSGIDLSSNGLTWVRQAPGEGLEWIGTINKDASAYYASWAKGRLTISKPSSTKVDLKITSPTTEDTATYFCGRIAFKTGTSIWGPGTLVTVSS

SEQ ID NO: 344 - protein, 112 aa, synthetic construct
  REGION 1..112: note = chemically synthesized
AIVMTQTPSPVSAAVGGTVTINCQASESVYSNNYLSWFQQKPGQPPKLLIYLASTLASGVPSRFKGSGSGTQFTLTISGVQCDDAATYYCIGGKSSSTDGNAFGGGTEVVVR

SEQ ID NO: 345 - protein, 121 aa, synthetic construct
  REGION 1..121: note = chemically synthesized
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARGNIVATITPLDYWGQGTLVTVSS

SEQ ID NO: 346 - protein, 110 aa, synthetic construct
  REGION 1..110: note = chemically synthesized
QPVLTQPPSVSAAPGQKVTISCSGSSSNIANNYVSWYQQLPGTAPKLLIFANNKRPSGIPDRFSGSKSGTSAALDITGLQTGDEADYYCGTWDSDLRAGVFGGGTKLTVL

SEQ ID NO: 347 - protein, 123 aa, synthetic construct; identical to SEQ ID NO: 243
  REGION 1..123: note = chemically synthesized

SEQ ID NO: 348 - protein, 118 aa, synthetic construct; identical to SEQ ID NO: 252
  REGION 1..118: note = chemically synthesized

SEQ ID NO: 349 - protein, 118 aa, synthetic construct; identical to SEQ ID NO: 253
  REGION 1..118: note = chemically synthesized

SEQ ID NO: 350 - protein, 118 aa, synthetic construct; identical to SEQ ID NO: 252
  REGION 1..118: note = chemically synthesized

SEQ ID NO: 351 - protein, 118 aa, synthetic construct; identical to SEQ ID NO: 253
  REGION 1..118: note = chemically synthesized

SEQ ID NO: 352 - protein, 118 aa, synthetic construct; identical to SEQ ID NO: 253
  REGION 1..118: note = chemically synthesized

SEQ ID NO: 353 - protein, 118 aa, synthetic construct; identical to SEQ ID NO: 252
  REGION 1..118: note = chemically synthesized

SEQ ID NO: 354 - protein, 120 aa, synthetic construct; identical to SEQ ID NO: 251
  REGION 1..120: note = chemically synthesized

SEQ ID NO: 355 - protein, 118 aa, synthetic construct; identical to SEQ ID NO: 261
  REGION 1..118: note = chemically synthesized

SEQ ID NO: 356 - protein, 118 aa, synthetic construct; identical to SEQ ID NO: 262
  REGION 1..118: note = chemically synthesized

SEQ ID NO: 357 - protein, 121 aa, synthetic construct
  REGION 1..121: note = chemically synthesized
QMQLVQSGAEVKKPGSSVKVSCKASGGTFSSYAYSWVRQAPGQGLEWMGGIIPSFGTANYAQKFQGRVTITADESTSTAYMELSSLRSEDTAVYYCARGPIVATITPLDYWGQGTLVTVSS

SEQ ID NO: 358 - protein, 121 aa, synthetic construct
  REGION 1..121: note = chemically synthesized
QMQLVQSGAEVKKPGSSVKVSCKASGGTFSSYAYSWVRQAPGQGLEWMGGIIPIFGTANYAQKFQGRVTITADESTSTAYMELSSLRSEDTAVYYCARGPIVATITPLDYWGQGTLVTVSS

SEQ ID NO: 359 - protein, 121 aa, synthetic construct; identical to SEQ ID NO: 357
  REGION 1..121: note = chemically synthesized

SEQ ID NO: 360 - protein, 121 aa, synthetic construct
  REGION 1..121: note = chemically synthesized
QMQLVQSGAEVKKPGSSVKVSCKASGGTFSSYAISWVRQAPGQGLEWMGGIIPAFGTANYAQKFQGRVTITADESTSTAYMELSSLRSEDTAVYYCARGPIVATITPLDYWGQGTLVTVSS

SEQ ID NO: 361 - protein, 108 aa, synthetic construct; identical to SEQ ID NO: 289
  REGION 1..108: note = chemically synthesized

SEQ ID NO: 362 - protein, 107 aa, synthetic construct
  REGION 1..107: note = chemically synthesized
AIRMTQSPSSLSASVGDRVTITCRASQSISSYLNWYQQKPGKAPKLLIYTTSSLKSGVPSRFSGSGSGTDFTLTISRLQPEDFATYYCQQSYSSTWTFGRGTKVEIK

SEQ ID NO: 363 - protein, 110 aa, synthetic construct; identical to SEQ ID NO: 263
  REGION 1..110: note = chemically synthesized

SEQ ID NO: 364 - protein, 110 aa, synthetic construct; identical to SEQ ID NO: 272
  REGION 1..110: note = chemically synthesized

SEQ ID NO: 365 - protein, 111 aa, synthetic construct; identical to SEQ ID NO: 273
  REGION 1..111: note = chemically synthesized

SEQ ID NO: 366 - protein, 110 aa, synthetic construct; identical to SEQ ID NO: 274
  REGION 1..110: note = chemically synthesized

SEQ ID NO: 367 - protein, 110 aa, synthetic construct; identical to SEQ ID NO: 275
  REGION 1..110: note = chemically synthesized

SEQ ID NO: 368 - protein, 110 aa, synthetic construct; identical to SEQ ID NO: 276
  REGION 1..110: note = chemically synthesized

SEQ ID NO: 369 - protein, 112 aa, synthetic construct; identical to SEQ ID NO: 277
  REGION 1..112: note = chemically synthesized

SEQ ID NO: 370 - protein, 110 aa, synthetic construct; identical to SEQ ID NO: 284
  REGION 1..110: note = chemically synthesized

SEQ ID NO: 371 - protein, 110 aa, synthetic construct; identical to SEQ ID NO: 286
  REGION 1..110: note = chemically synthesized

SEQ ID NO: 372 - protein, 112 aa, synthetic construct; identical to SEQ ID NO: 287
  REGION 1..112: note = chemically synthesized

SEQ ID NO: 373 - protein, 108 aa, synthetic construct; identical to SEQ ID NO: 288
  REGION 1..108: note = chemically synthesized

SEQ ID NO: 374 - protein, 108 aa, synthetic construct; identical to SEQ ID NO: 289
  REGION 1..108: note = chemically synthesized

SEQ ID NO: 375 - protein, 108 aa, synthetic construct; identical to SEQ ID NO: 289
  REGION 1..108: note = chemically synthesized

SEQ ID NO: 376 - protein, 108 aa, synthetic construct; identical to SEQ ID NO: 289
  REGION 1..108: note = chemically synthesized

SEQ ID NO: 377 - protein, 122 aa, synthetic construct
  REGION 1..122: note = chemically synthesized
QVQLVQSGSEVKKSGSSVKVSCKTSGGTFSITNYAINWVRQAPGQGLEWMGGILPIFGAAKYAQKFQDRVTITADESTNTAYLELSSLTSEDTAMYYCARGKRWLQSDLQYWGQGTLVTVSS

SEQ ID NO: 378 - protein, 110 aa, synthetic construct
  REGION 1..110: note = chemically synthesized
QPVLTQPASVSGSPGQSITISCTGSSSDVGSYDLVSWYQQSPGKVPKLLIYEGVKRPSGVSNRFSGSKSGNTASLTISGLQAEDEADYYCSSYAGTRNFVFGGGTQLTVL

SEQ ID NO: 379 - protein, 121 aa, synthetic construct
  REGION 1..121: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKSSAGQSRPGFDYWGQGTLVTVSS

SEQ ID NO: 380 - protein, 121 aa, synthetic construct
  REGION 1..121: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKSSAGQSWPGFDYWGQGTLVTVSS

SEQ ID NO: 381 - protein, 121 aa, synthetic construct
  REGION 1..121: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKSSAGQSFPGFDYWGQGTLVTVSS

SEQ ID NO: 382 - protein, 116 aa, synthetic construct
  REGION 1..116: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKWSAAFDYWGQGTLVTVSS

SEQ ID NO: 383 - protein, 116 aa, synthetic construct
  REGION 1..116: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKWSAGYDYWGQGTLVTVSS

SEQ ID NO: 384 - protein, 116 aa, synthetic construct
  REGION 1..116: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKWSKGFDYWGQGTLVTVSS

SEQ ID NO: 385 - protein, 113 aa, synthetic construct
  REGION 1..113: note = chemically synthesized
EVQLLESGGGLVQPGGSLRLSCAASGFTFSSYAMSWVRQAPGKGLEWVSSIWKQGIVTVYDSVKGRFTISRDNSKNTLYLQMNSLRAEDTAVYYCAKSSAGFDYWGQGTLVTV

SEQ ID NO: 386 - protein, 115 aa, synthetic construct
  REGION 1..115: note = chemically synthesized
EVQLLESGGGLVQPGGSLRLSCAASGFTFSSYAMSWVRQAPGKGLEWVSSIWRNGIVTVYDSVKGRFTISRDNSKNTLYLQMNSLRAEDTAVYYCAKSSAGFDYWGQGTLVTVSS

SEQ ID NO: 387 - protein, 115 aa, synthetic construct
  REGION 1..115: note = chemically synthesized
EVQLLESGGGLVQPGGSLRLSCAASGFTFSSYAMSWVRQAPGKGLEWVSDIWKQGMVTVYDSVKGRFTISRDNSKNTLYLQMNSLRAEDTAVYYCAKSSAGFDYWGQGTLVTVSS

SEQ ID NO: 388 - protein, 115 aa, synthetic construct
  REGION 1..115: note = chemically synthesized
EVQLLESGGGLVQPGGSLRLSCAASGFTFSSYAMSWVRQAPGKGLEWVSSIWRQGLATAYDSVKGRFTISRDNSKNTLYLQMNSLRAEDTAVYYCAKSSAGFDYWGQGTLVTVSS

SEQ ID NO: 389 - protein, 115 aa, synthetic construct
  REGION 1..115: note = chemically synthesized
EVQLLESGGGLVQPGGSLRLSCAASGFTFSSYAMSWVRQAPGKGLEWVSEIVATGILTSYDSVKGRFTISRDNSKNTLYLQMNSLRAEDTAVYYCAKSSAGFDYWGQGTLVTVSS

SEQ ID NO: 390 - protein, 115 aa, synthetic construct
  REGION 1..115: note = chemically synthesized
EVQLLESGGGLVQPGGSLRLSCAASGFTFSSYAMSWVRQAPGKGLEWVSSIGRQGLITVYDSVKGRFTISRDNSKNTLYLQMNSLRAEDTAVYYCAKSSAGFDYWGQGTLVTVSS

SEQ ID NO: 391 - protein, 115 aa, synthetic construct
  REGION 1..115: note = chemically synthesized
EVQLLESGGGLVQPGGSLRLSCAASGFTFSSYAMSWVRQAPGKGLEWVSSIWYQGLVTVYDSVKGRFTISRDNSKNTLYLQMNSLRAEDTAVYYCAKSSAGFDYWGQGTLVTVSS

SEQ ID NO: 392 - protein, 114 aa, synthetic construct
  REGION 1..114: note = chemically synthesized
EVQLLESGGGLVQPGGSLRLSCAASGFTFSSYAMSWVRQAPGKGLEWVSDIWKQGFATADSVKGRFTISRDNSKNTLYLQMNSLRAEDTAVYYCAKSSAGFDYWGQGTLVTVSS

SEQ ID NO: 393 - protein, 115 aa, synthetic construct
  REGION 1..115: note = chemically synthesized
EVQLLESGGGLVQPGGSLRLSCAASGFTFSSYAMSWVRQAPGKGLEWVSSIWKQGIVTVYDSVKGRFTISRDNSKNTLYLQMNSLRAEDTAVYYCAKSSAGFDYWGQGTLVTVSS

SEQ ID NO: 394 - protein, 115 aa, synthetic construct; identical to SEQ ID NO: 388
  REGION 1..115: note = chemically synthesized

SEQ ID NO: 395 - protein, 116 aa, synthetic construct
  REGION 1..116: note = chemically synthesized
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSS

SEQ ID NO: 396 - protein, 116 aa, synthetic construct
  REGION 1..116: note = chemically synthesized
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAGYDYWGQGTLVTVSS

SEQ ID NO: 397 - protein, 116 aa, synthetic construct
  REGION 1..116: note = chemically synthesized
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSKGFDYWGQGTLVTVSS

SEQ ID NO: 398 - protein, 120 aa, synthetic construct
  REGION 1..120: note = chemically synthesized
EVQLLESGGGLVQPGGSLRLSCAASGFTFSSYAMETSWVRQAPGKGLEWVSSIWYQGLVTVYADSVKGRFTISRDNSKNTLYLQMETNSLRAEDTAVYYCAKWSAAFDYWGQGTLVTVSS

SEQ ID NO: 399 - protein, 116 aa, synthetic construct
  REGION 1..116: note = chemically synthesized
EVQLLESGGGLVQPGGSLRLSCAASGFTFSSYAMSWVRQAPGKGLEWVSSIWYQGLVTVYADSVKGRFTISRDNSKNTLYLQMNSLRAEDTAVYYCAKWSAGYDYWGQGTLVTVSS

SEQ ID NO: 400 - protein, 116 aa, synthetic construct
  REGION 1..116: note = chemically synthesized
EVQLLESGGGLVQPGGSLRLSCAASGFTFSSYAMSWVRQAPGKGLEWVSSIWYQGLVTVYADSVKGRFTISRDNSKNTLYLQMNSLRAEDTAVYYCAKWSKGFDYWGQGTLVTVSS

SEQ ID NO: 401 - protein, 108 aa, synthetic construct
  REGION 1..108: note = chemically synthesized
DIQMTQSPSSLSASVGDRVTITCRASQSISSYLNWYQQKPGKAPKLLIYYASTLQSGVPSRFSGSGSGTDFTLTISSLQPEDFATYYCQQDNGYPSTFGQGTKVEIKR

SEQ ID NO: 402 - protein, 108 aa, synthetic construct; identical to SEQ ID NO: 401
  REGION 1..108: note = chemically synthesized

SEQ ID NO: 403 - protein, 108 aa, synthetic construct
  REGION 1..108: note = chemically synthesized
DIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKR

SEQ ID NO: 404 - protein, 240 aa, synthetic construct
  REGION 1..240: note = chemically synthesized
EVQLLESGGGLVQPGGSLRLSCAASGFTFSSYAMSWVRQAPGKGLEWVSDITASGQRTTYADSVKGRFTISRDNSKNTLYLQMNSLRAEDTAVYYCARSKIAFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQRALKPVTFGQGTKVEIKR

SEQ ID NO: 405 - protein, 240 aa, synthetic construct
  REGION 1..240: note = chemically synthesized
EVQLLESGGGLVQPGGSLRLSCAASGFTFSSYAMSWVRQAPGKGLEWVSSINKDGHYTSYADSVKGRFTISRDNSKNTLYLQMNSLRAEDTAVYYCAKNLDEFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 406 - protein, 240 aa, synthetic construct
  REGION 1..240: note = chemically synthesized
EVQLLESGGGLVQPGGSLRLSCAASGFTFSSYAMSWVRQAPGKGLEWVSSIMATGAGTLYADSVKGRFTISRDNSKNTLYLQMNSLRAEDTAVYYCAKDGAGFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISSLQPEDFATYYCQQANSRPSTFGQGTKVEIKR

SEQ ID NO: 407 - protein, 240 aa, synthetic construct
  REGION 1..240: note = chemically synthesized
EVQLLESGGGLVQPGGSLRLSCAASGFTFSSYAMSWVRQAPGKGLQWVSTITSSGAATYYADSVKGRFTISRDNSKNTLYLQMNSLRAEDTAVYYCAKNYTGFDYWGQGTLVTVSSGGGGSGGGGSGGGGSTDIQMTQSPSSLSASVGDRVTITCRASQSISSYLNWYQQKPGKAPKLLIYNASSLQSGVPSRFSGSGSGTDFTLTISSLQPEDFATYYCQQYTYGPGTFGQGTKVEIKR

SEQ ID NO: 408 - protein, 240 aa, synthetic construct
  REGION 1..240: note = chemically synthesized
EVQLLESGGGLVQPGGSLRLSCAASGFTFSSYAMSWVRQAPGKGLEWVSSIYSTGGATAYADSVKGRFTISRDNSKNTLYLQMNSLRAEDTAVYYCAKSSAGFDYWGQGTLVTVSSGGGGSGGGGSGGGGSTDIQMTQSPSSLSASVGDRVTITCRASQSISSYLNWYQQKPGKAPKLLIYYASTLQSGVPSRFSGSGSGTDFTLTISSLQPEDFATYYCQQDNGYPSTFGQGTKVEIKR

SEQ ID NO: 409 - protein, 122 aa, synthetic construct
  REGION 1..122: note = chemically synthesized
QLQLQESGGGLVQAGGSLRLSCAASGIMFYISDMGWYRQAPGKQREFVATITSGGTTNYADSVEGRFSISRDNAKNTVYLQMNSLEPEDTAVYYCTAHGPTYGSTWDDLWGQGTQVTVKPGG

SEQ ID NO: 410 - protein, 8 aa, synthetic construct
  REGION 1..8: note = chemically synthesized
GIMFYISD

SEQ ID NO: 411 - protein, 10 aa, synthetic construct
  REGION 1..10: note = chemically synthesized
TITSGGTTNY

SEQ ID NO: 412 - protein, 14 aa, synthetic construct
  REGION 1..14: note = chemically synthesized
TAHGPTYGSTWDDL

SEQ ID NO: 413 - protein, 113 aa, synthetic construct
  REGION 1..113: note = chemically synthesized
QVQLQESGGGLVQAGGSLRLSCAASETFGVVFTLGWYRQTPGKQREFVARVTGTDTVDYADSVKGRFTISSDFARNTVYLQMNNLKPEDTAVYYCNTGAYWGQGTQVTVKPGG

SEQ ID NO: 414 - protein, 7 aa, synthetic construct
  REGION 1..7: note = chemically synthesized
TFGVVFT

SEQ ID NO: 415 - protein, 7 aa, synthetic construct
  REGION 1..7: note = chemically synthesized
VTGTDTV

SEQ ID NO: 416 - protein, 5 aa, synthetic construct
  REGION 1..5: note = chemically synthesized
NTGAY

SEQ ID NO: 417 - protein, 119 aa, synthetic construct
  REGION 1..119: note = chemically synthesized
QVQLVQSGGGLVQTGGSLRLSCAASGRTASTYSMGWFRQAPGKERQFVARIIWSTGSTYYTNSVEGRFTISRDIAKNTLYLQMNSLEPEDTAVYYCTAREPTGYDYWGQGTQVTVKPGG

SEQ ID NO: 418 - protein, 8 aa, synthetic construct
  REGION 1..8: note = chemically synthesized
GRTASTYS

SEQ ID NO: 419 - protein, 7 aa, synthetic construct
  REGION 1..7: note = chemically synthesized
IWSTGST

SEQ ID NO: 420 - protein, 10 aa, synthetic construct
  REGION 1..10: note = chemically synthesized
TAREPTGYDY

SEQ ID NO: 421 - protein, 122 aa, synthetic construct
  REGION 1..122: note = chemically synthesized
QLQLQESGGGLVQAGGSLGLSCAASGSIFRFGARGWYRQAPGKQRELVAIITSGGSTNYADSVQGRFTISRDNAKNMVYLQMNGLKSGDTAVYYCAADRSDAVGVGWDYWGQGTQVTVKPGG

SEQ ID NO: 422 - protein, 8 aa, synthetic construct
  REGION 1..8: note = chemically synthesized
GSIFRFGA

SEQ ID NO: 423 - protein, 7 aa, synthetic construct
  REGION 1..7: note = chemically synthesized
ITSGGST

SEQ ID NO: 424 - protein, 14 aa, synthetic construct
  REGION 1..14: note = chemically synthesized
AADRSDAVGVGWDY

SEQ ID NO: 425 - protein, 119 aa, synthetic construct
  REGION 1..119: note = chemically synthesized
QVQLQQSGGGLVQTGGSLRLSCAASGRTASTYSMGWFRQAPGKERQFVARIIWSTGSTYYTNSVEGRFTISRDIAKNTLYLQMNSLEPEDTAVYYCTARDPTGYDYWGQGTQVTVKPGG

SEQ ID NO: 426 - protein, 8 aa, synthetic construct
  REGION 1..8: note = chemically synthesized
IIWSTGST

SEQ ID NO: 427 - protein, 10 aa, synthetic construct
  REGION 1..10: note = chemically synthesized
TARDPTGYDY

SEQ ID NO: 428 - protein, 121 aa, synthetic construct
  REGION 1..121: note = chemically synthesized
QLQLQESGGGLVQAGGSLRLSCAASGSIFSIDATAWYRQAPGKQRELVAIITSSGSTNYPDSVKGRFTISRDNAKNTVYLQMNSLNPEDTALYSCNAITRMGGSTYDFWGQGTQVTVKPGG

SEQ ID NO: 429 - protein, 8 aa, synthetic construct
  REGION 1..8: note = chemically synthesized
GSIFSIDA

SEQ ID NO: 430 - protein, 7 aa, synthetic construct
  REGION 1..7: note = chemically synthesized
ITSSGST

SEQ ID NO: 431 - protein, 13 aa, synthetic construct
  REGION 1..13: note = chemically synthesized
NAITRMGGSTYDF

SEQ ID NO: 432 - protein, 117 aa, synthetic construct
  REGION 1..117: note = chemically synthesized
QVQLVQSGGGLVQPGGSLRLSCAASGFTFSSYAMSWVRQAPGKGLEWVSAIPAGDGSTKYADSVKGRFTISRDNAKNTVYLQMDSLKPEDTAVYFCAKSRGWSTVDDMDYWGKGTQV

SEQ ID NO: 433 - protein, 8 aa, synthetic construct
  REGION 1..8: note = chemically synthesized
GFTFSSYA

SEQ ID NO: 434 - protein, 8 aa, synthetic construct
  REGION 1..8: note = chemically synthesized
IPAGDGST

SEQ ID NO: 435 - protein, 14 aa, synthetic construct
  REGION 1..14: note = chemically synthesized
AKSRGWSTVDDMDY

SEQ ID NO: 436 - protein, 117 aa, synthetic construct
  REGION 1..117: note = chemically synthesized
QVQLVQSGGGLVQPGGSLRLSCVVSGFTFRSYAMSWVRQAPGKGLEWVSTINSGESSTKYADSVKGRFTISRDDAKNTLYLQMSDLKPEDTAVYFCAKHRGWSTVDDINYWGKGTQV

SEQ ID NO: 437 - protein, 8 aa, synthetic construct
  REGION 1..8: note = chemically synthesized
GFTFRSYA

SEQ ID NO: 438 - protein, 8 aa, synthetic construct
  REGION 1..8: note = chemically synthesized
INSGESST

SEQ ID NO: 439 - protein, 14 aa, synthetic construct
  REGION 1..14: note = chemically synthesized
AKHRGWSTVDDINY

SEQ ID NO: 440 - protein, 119 aa, synthetic construct
  REGION 1..119: note = chemically synthesized
QVQLVQSGGGLVQPGGSLRLSCAASGFTFDDHAMSWVRQAPGKGLEWVSAISWNGHYTYYAESMKGRFAISRDNAKNTLYLQMNSLKSEDTAVYYCVKGWRGSYTRDRPFASWGQGTQV

SEQ ID NO: 441 - protein, 8 aa, synthetic construct
  REGION 1..8: note = chemically synthesized
GFTFDDHA

SEQ ID NO: 442 - protein, 8 aa, synthetic construct
  REGION 1..8: note = chemically synthesized
ISWNGHYT

SEQ ID NO: 443 - protein, 16 aa, synthetic construct
  REGION 1..16: note = chemically synthesized
VKGWRGSYTRDRPFAS

SEQ ID NO: 444 - protein, 118 aa, synthetic construct
  REGION 1..118: note = chemically synthesized
EVQLVQSGGGLVQPGGSLRLSCAASGFTFSSYYMSWVRQAPGKGLEWVSTISTNTGGGSTYYAYADSVKGRFTISRDNAKNTLYLEMNSLKPEDTAQYYCVRTRWEGVYDYWGLGTQV

SEQ ID NO: 445 - protein, 8 aa, synthetic construct
  REGION 1..8: note = chemically synthesized
GFTFSSYY

SEQ ID NO: 446 - protein, 10 aa, synthetic construct
  REGION 1..10: note = chemically synthesized
ISTNTGGGST

SEQ ID NO: 447 - protein, 11 aa, synthetic construct
  REGION 1..11: note = chemically synthesized
VRTRWEGVYDY

SEQ ID NO: 448 - protein, 482 aa, synthetic construct
  REGION 1..482: note = chemically synthesized
EVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSEVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKPGG

SEQ ID NO: 449 - protein, 466 aa, synthetic construct
  REGION 1..466: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGFSFSINAMGWYRQAPGKRREFVAAIESGRNTVYAESVKGRFTISRDNAKNTVYLQMSSLRAEDTAVYYCGLLKGNRVVSPSVAYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 450 - protein, 466 aa, synthetic construct
  REGION 1..466: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGFSFSINAMGWYRQAPGKRREFVAAIYSGRNTVYAESVKGRFTISRDNAKNTVYLQMSSLRAEDTAVYYCGLLKGNRVVSPSVAYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 451 - protein, 466 aa, synthetic construct
  REGION 1..466: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQPGGSLRLSCAASGFSFSINAMGWYRQAPGKRREFVAAIYSGSSTVYAESVKGRFTISRDNAKNTVYLQMSSLRAEDTAVYYCGLLKGNRVVSPSVAYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 452 - protein, 481 aa, synthetic construct
  REGION 1..481: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQLLESGGGEVQPGGSLRLSCAASGWAFGNYGMAWFRQAPGKEREFVSRLAWQGGSTDYVESVKGRFTISRDNAKNTLYLQMSSLRAEDTAVYYCARQRSYSRYDIRTPQTYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 453 - protein, 481 aa, synthetic construct
  REGION 1..481: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQLLESGGGEVQPGGSLRLSCAASGWAFGNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 454 - protein, 481 aa, synthetic construct
  REGION 1..481: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQLLESGGGEVQPGGSLRLSCAASGWAFSNYGMAWFRQAPGKEREFVSRLAWGGGSTDYVESVKGRFTISRDNAKNTLYLQMSSLRAEDTAVYYCARQRSYSRYDIRTPQTYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - protein, 481 aa, synthetic construct
  REGION 1..481: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQLLESGGGEVQPGGSLRLSCAASGWAFGNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 456 - protein, 481 aa, synthetic construct
  REGION 1..481: note = chemically synthesized
EVQLLESGGGEVQPGGSLRLSCAASGGIFAIKPISWYRQAPGKQREWVSTTTSSGATNYAESVKGRFTISRDNAKNTLYLQMSSLRAEDTAVYYCNVFEYWGQGTLVTVKPGGSGGSEVQLLESGGGEVQLLESGGGEVQPGGSLRLSCAASGWAFSNYGMAWFRQAPGKEREFVSRLAWSGGSTDYVESVKGRFTISRDNAKNTLYLQMSSLRAEDTAVYYCARQRSYSRYDIRTPQTYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0250349 A1
(43) Pub. Date: Aug. 7, 2025

(54) USES OF PDL1-BINDING PROTEINS

(71) Applicant: Inhibrx Biosciences, Inc., La Jolla, CA (US)

(72) Inventors: Brendan P. Eckelman, Encinitas, CA (US); John C. Timmer, San Diego, CA (US); Chelsie Macedo, La Jolla, CA (US); Kyle S. Jones, San Marcos, CA (US); Abrahim Hussain, La Jolla, CA (US); Amir S. Razai, La Jolla, CA (US); Bryan R. Becklund, San Diego, CA (US); Rajay A. Pandit, La Jolla, CA (US); Mike Kaplan, La Jolla, CA (US); Lucas Rascon, La Jolla, CA (US); Quinn Deveraux, La Jolla, CA (US)

(73) Assignee: Inhibrx Biosciences, Inc., La Jolla, CA (US)

(21) Appl. No.: 18/956,693

(22) Filed: Nov. 22, 2024

Related U.S. Application Data
(62) Division of application No. 18/067,484, filed on Dec. 16, 2022, now Pat. No. 12,187,804.
Application No. 18/067,484 is a division of application No. 16/601,825, filed on Oct. 15, 2019, now Pat. No. 11,566,078.
Application No. 16/601,825 is a division of application No. 15/404,016, filed on Jan. 11, 2017, now Pat. No. 10,501,551.
(60) Provisional application No. 62/277,028, filed on Jan. 11, 2016.

(51) Int. Cl. C07K 16/28

(52) U.S. Cl. CPC C07K 16/2878; C07K 16/2827; C07K 2317/24; C07K 2317/31; C07K 2317/33; C07K 2317/35; C07K 2317/52; C07K 2317/569; C07K 2317/75; C07K 2317/76; C07K 2317/92; C07K 2319/00

(57) ABSTRACT

This invention relates generally to molecules that specifically engage 41BB, a member of the TNF receptor superfamily (TNFRSF). More specifically, this invention relates to multivalent and multispecific molecules that bind at least 41BB.

### RELATED APPLICATIONS

This application is a divisional of U.S. patent application Ser. No. 18/067,484, filed Dec. 16, 2022, which is a divisional of U.S. patent application Ser. No. 16/601,825, filed Oct. 15, 2019, issued as U.S. Pat. No. 11,566,078, which is a divisional of U.S. patent application Ser. No. 15/404,016, filed Jan. 11, 2017, issued as U.S. Pat. No. 10,501,551, which claims the benefit of U.S. Provisional Application No. 62/277,028, filed Jan. 11, 2016; the contents of each of which are incorporated herein by reference in their entirety.

### SEQUENCE LISTING

The present application contains a Sequence Listing which has been submitted electronically in XML format and is hereby incorporated by reference in its entirety. Said XML copy, created on Dec. 14, 2022, is named “2022-12-14_01202-0005-02US.xml” and is 617,844 bytes in size. The information in the electronic format of the sequence listing is incorporated herein by reference in its entirety.

### FIELD OF THE INVENTION

This invention relates generally to molecules that specifically engage 41BB, a member of the TNF receptor superfamily (TNFRSF). More specifically, this invention relates to multivalent and/or multispecific molecules that bind at least 41BB.

### BACKGROUND OF THE INVENTION

The tumor necrosis factor receptor superfamily consists of several structurally related cell surface receptors. Activation by multimeric ligands is a common feature of many of these receptors. Many members of the TNFRSF have therapeutic utility in numerous pathologies, if activated properly. Agonism of this receptor family often requires higher order clustering, and conventional bivalent antibodies are not ideal for this purpose. Therefore, there exists a therapeutic need for more potent agonist molecules of the TNFRSF.

### SUMMARY OF THE INVENTION

The disclosure provides multivalent and multispecific TNF receptor superfamily (TNFRSF) binding fusion polypeptides that bind at least 41BB (also known as tumor necrosis factor receptor superfamily, member 4 (TNFRSF9) and/or CD137)). The use of the term “41BB” is intended to cover any variation thereof, such as, by way of non-limiting example, 41-BB and/or 4-1BB, and all variations are used herein interchangeably. These molecules that bind at least 41BB are referred to herein as “41BB-targeting molecules” or “41BB-targeting fusions” or “41BB-targeting proteins” or “41BB-targeting fusion polypeptides” or “41BB-targeting fusion proteins.” In some embodiments, the 41BB-targeting molecule is a multivalent molecule, for example, a multivalent 41BB-targeting fusion protein. In some embodiments, the 41BB-targeting molecule is a multispecific molecule, for example, a multispecific 41BB-targeting fusion protein. In some embodiments, the 41BB-targeting molecule is a multivalent and multispecific molecule, for example, a multivalent and multispecific 41BB-targeting fusion protein. As used herein, the term “fusion protein” or “fusion polypeptide” or “41BB-targeting fusion protein” or “41BB-targeting fusion polypeptide,” unless otherwise specifically denoted, refers to any fusion protein embodiment of the disclosure, including, but not limited to, multivalent fusion proteins, multispecific fusion proteins, or multivalent and multispecific fusion proteins.

The disclosure also provides multivalent and multispecific fusion polypeptides that bind at least programmed death ligand 1 (PDL1), also known as PD-L1, CD274, B7 homolog 1 and/or B7-H1. The use of the term “PDL1” is intended to cover any variation thereof, such as, by way of non-limiting example, PD-L1 and/or PDL-1, all variations are used herein interchangeably. These molecules that bind at least PDL1 are referred to herein as “PDL1-targeting molecules” or “PDL1-targeting fusions” or “PDL1-targeting proteins” or “PDL1-targeting fusion polypeptides” or “PDL1-targeting fusion proteins.” In some embodiments, the PDL1-targeting molecule is a multivalent molecule, for example, a multivalent PDL1-targeting fusion protein. In some embodiments, the PDL1-targeting molecule is a multispecific molecule, for example, a multispecific PDL1-targeting fusion protein. In some embodiments, the PDL1-targeting molecule is a multivalent and multispecific molecule, for example, a multivalent and multispecific PDL1-targeting fusion protein. As used herein, the term “fusion protein” or “fusion polypeptide” or “PDL1-targeting fusion protein” or “PDL1-targeting fusion polypeptide,” unless otherwise specifically denoted, refers to any fusion protein embodiment of the disclosure, including, but not limited to, multivalent fusion proteins, multispecific fusion proteins, or multivalent and multispecific fusion proteins.

The disclosure also provides multivalent and multispecific fusion polypeptides that bind at least PDL1 and 41BB. These molecules that bind at least PDL1 are referred to herein as “PDL1×41BB-targeting molecules” or “PDL1×41BB-targeting fusions” or “PDL1×41BB-targeting proteins” or “PDL1×41BB-targeting fusion polypeptides” or “PDL1×41BB-targeting fusion proteins.” In some embodiments, the PDL1×41BB-targeting molecule is a multivalent molecule, for example, a multivalent PDL1×41BB-targeting fusion protein. In some embodiments, the PDL1×41BB-targeting molecule is a multispecific molecule, for example, a multispecific PDL1×41BB-targeting fusion protein. In some embodiments, the PDL1×41BB-targeting molecule is a multivalent and multispecific molecule, for example, a multivalent and multispecific PDL1-targeting fusion protein. As used herein, the term “fusion protein” or “fusion polypeptide” or “PDL1×41BB-targeting fusion protein” or “PDL1×41BB-targeting fusion polypeptide,” unless otherwise specifically denoted, refers to any fusion protein embodiment of the disclosure, including, but not limited to, multivalent fusion proteins, multispecific fusion proteins, or multivalent and multispecific fusion proteins.

In some embodiments, the multivalent and/or multispecific fusion protein binds at least 41BB. Conventional antibodies targeting members of the TNF receptor superfamily (TNFRSF) have been shown to require exogenous crosslinking to achieve sufficient agonist activity, as evidenced by the necessity for Fc-gamma Receptor (FcγRs) for the activity of antibodies to DR4, DR5, GITR and OX40 (Ichikawa et al 2001 al Nat. Med. 7, 954-960, Li et al 2008 Drug Dev. Res. 69, 69-82; Pukac et al 2005 Br. J. Cancer 92, 1430-1441; Yanda et al 2008 Ann. Oncol. 19, 1060-1067; Yang et al 2007 Cancer Lett. 251:146-157; Bulliard et al 2013 JEM 210(9): 1685; Bulliard et al 2014 Immunol and Cell Biol 92: 475-480). In addition to crosslinking via FcγRs other exogenous agents including addition of the oligomeric ligand or antibody binding entities (e.g. protein A and secondary antibodies) have been demonstrated to enhance anti-TNFRSF antibody clustering and downstream signaling. For example, the addition of the DR5 ligand TRAIL enhanced the apoptosis inducing ability of an anti-DR5 antibody (Graves et al 2014 Cancer Cell 26: 177-189). These findings suggest the need for clustering of TNFRSFs beyond a dimer.

The present disclosure provides multivalent TNFRSF binding fusion proteins, which comprise 2 or more TNFRSF binding domains (TBDs) where at least one TBD binds 41BB. In some embodiments, the fusion proteins of the present disclosure have utility in treating neoplasms.

In some embodiments, the fusion protein contains two or more different TBDs, where each TBD binds 41BB. In some embodiments, the fusion protein contains multiple copies of a TBD that binds 41BB. For example, in some embodiments, the fusion protein contains at least two copies of a TBD that binds 41BB. In some embodiments, the fusion protein contains at least three copies of a TBD that binds 41BB. In some embodiments, the fusion protein contains at least four copies of a TBD that binds 41BB. In some embodiments, the fusion protein contains at least five copies of a TBD that binds 41BB. In some embodiments, the fusion protein contains at least six copies of a TBD that binds 41BB. In some embodiments, the fusion protein contains six or more copies of a TBD that binds 41BB.

In other embodiments, the fusion proteins of the present disclosure bind 41BB and a second TNFRSF member for example GITR, OX40, CD27, TNFR2 and/or CD40. In these embodiments, the fusion proteins of the present disclosure modulate immune cells leading to enhanced tumor destruction. In other embodiments, the fusion proteins of the present disclosure have utility in treating inflammatory conditions. In these embodiments, the fusion proteins of the present disclosure modulate immune cells leading to dampening of the inflammatory insult. For example, specifically agonizing TNFR2 can enhance Treg proliferation leading to immune suppression.

The fusion proteins of the present disclosure are capable of enhanced clustering of TNFRSF members compared to non-cross-linked bivalent antibodies. The enhanced clustered of TNFRSF members mediated by the fusion proteins of the present disclosure induce enhanced TNFRSF-dependent signaling compared to non-cross-linked bivalent antibodies. In most embodiments, the fusion protein will incorporate more than 2 TBDs, for example, three, four, five, or six.

In some embodiments, the fusion proteins are multispecific containing a TBD and a binding domain directed toward a second antigen. In these, embodiments, the binding to the second antigen is capable of providing the additional crosslinking function and TNFRSF activation can be achieved with only one or two TBDs. In these embodiments, the TNFRSF signaling is enhanced and focused by the presence of the second antigen. These multispecific TBD containing fusion proteins are useful means to achieve conditional signaling of a given TNFRSF member.

In these embodiments, binding to the TNFRSF member by the TBD induces minimal signaling unless the second antigen is co-engaged. For example, the multispecific fusion proteins of the present disclosure are capable binding 41BB and PD-L1 and 41BB-dependent signaling is greatly enhanced when the fusion protein is bound to a PD-L1 expressing cell. In another example, the multispecific fusion proteins of the present disclosure are capable binding 41BB and Folate Receptor Alpha (FRα) and 41BB-dependent signaling is greatly enhanced when the fusion protein is bound to a FRα expressing cell.

The present disclosure provides isolated polypeptides that specifically bind 41BB. In some embodiments, the isolated polypeptide is derived from antibodies or antibody fragments including scFv, Fabs, single domain antibodies (sdAb), V_{NAR}, or VHHs. In some embodiments, the isolated polypeptide is human or humanized sdAb. The sdAb fragments can be derived from VHH, V_{NAR}, engineered VH or VK domains. VHHs can be generated from camelid heavy chain only antibodies. V_{NARS} can be generated from cartilaginous fish heavy chain only antibodies. Various methods have been implemented to generate monomeric sdAbs from conventionally heterodimeric VH and VK domains, including interface engineering and selection of specific germline families. In other embodiments, the isolated polypeptides are derived from non-antibody scaffold proteins for example but not limited to designed ankyrin repeat proteins (darpins), avimers, anticalin/lipocalins, centyrins and fynomers.

In some embodiments, the isolated polypeptide includes an amino acid sequence selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83. In some embodiments, the isolated polypeptide includes an amino acid sequence selected from the group consisting of SEQ ID NO: 33, 39, 33-41, 43, 45-47, 49, 51, 53, 54, 56, 58-60, 62, 65, 66, 68, 70, 72, 74, 76, 78, and 80-83.

In some embodiments, the isolated polypeptide includes an amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83. In some embodiments, the isolated polypeptide includes an amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence selected from the group consisting of SEQ ID NO: 33, 39, 33-41, 43, 45-47, 49, 51, 53, 54, 56, 58-60, 62, 65, 66, 68, 70, 72, 74, 76, 78, and 80-83.

In some embodiments, the isolated polypeptide comprises a complementarity determining region 1 (CDR1) comprising an amino acid sequence selected from the group consisting of SEQ ID NO: 17, 21, 26, 30, 50, 65, and 69; a complementarity determining region 2 (CDR2) comprising an amino acid sequence selected from the group consisting of SEQ ID NO: 18, 27, 31, 42, 44, 48, 52, 61, 63, 71, 73, 75, 77, and 79; and a complementarity determining region 3 (CDR3) comprising an amino acid sequence selected from the group consisting of SEQ ID NO: 19, 22, 24, 28, 32, 55, and 57.

The present disclosure provides multivalent fusion proteins, which comprise two or more binding domains (BDs) where at least one BD binds PDL1. In some embodiments, the fusion proteins of the present disclosure have utility in treating neoplasms.

In some embodiments, the fusion protein contains two or more different BDs, where each BD binds PDL1. In some embodiments, the fusion protein contains multiple copies of a BD that binds PDL1. For example, in some embodiments, the fusion protein contains at least two copies of a BD that binds PDL1. In some embodiments, the fusion protein contains at least three copies of a BD that binds PDL1. In some embodiments, the fusion protein contains at least four copies of a BD that binds PDL1. In some embodiments, the fusion protein contains at least five copies of a BD that binds PDL1. In some embodiments, the fusion protein contains at least six copies of a BD that binds PDL1. In some embodiments, the fusion protein contains six or more copies of a BD that binds PDL1.

The present disclosure provides isolated polypeptides that specifically bind 41BB. In some embodiments, the isolated polypeptide is derived from antibodies or antibody fragments including scFv, Fabs, single domain antibodies (sdAb), V_{NAR}, or VHHs. In some embodiments, the isolated polypeptide is human or humanized sdAb. The sdAb fragments can be derived from VHH, V_{NAR}, engineered VH or VK domains. VHHs can be generated from camelid heavy chain only antibodies. V_{NARS} can be generated from cartilaginous fish heavy chain only antibodies. Various methods have been implemented to generate monomeric sdAbs from conventionally heterodimeric VH and VK domains, including interface engineering and selection of specific germline families. In other embodiments, the isolated polypeptides are derived from non-antibody scaffold proteins for example but not limited to designed ankyrin repeat proteins (darpins), avimers, anticalin/lipocalins, centyrins and fynomers.

In some embodiments, the isolated polypeptide includes an amino acid sequence selected from the group consisting of SEQ ID NO: 100, 104, 108, 112, 114, 116, and 119-124. In some embodiments, the isolated polypeptide includes an amino acid sequence selected from the group consisting of SEQ ID NO: 119-124.

In some embodiments, the isolated polypeptide includes an amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence selected from the group consisting of SEQ ID NO: 100, 104, 108, 112, 114, 116, and 119-124. In some embodiments, the isolated polypeptide includes an amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence selected from the group consisting of SEQ ID NO: 119-124.

In some embodiments, the isolated polypeptide comprises a complementarity determining region 1 (CDR1) comprising an amino acid sequence selected from the group consisting of SEQ ID NO: 101, 105, and 109; a complementarity determining region 2 (CDR2) comprising an amino acid sequence selected from the group consisting of SEQ ID NO: 102, 106, 110, and 117; and a complementarity determining region 3 (CDR3) comprising an amino acid sequence selected from the group consisting of SEQ ID NO: 103, 107, 111, 113, 115, and 118.

In some embodiments, the present disclosure provides isolated polypeptides that specifically bind at least 41BB and PDL1. In some embodiments, each binding domain (BD) in the isolated polypeptide is derived from antibodies or antibody fragments including scFv, Fabs, single domain antibodies (sdAb), V_{NAR}, or VHHs. In some embodiments, each BD is human or humanized sdAb. The sdAb fragments can be derived from VHH, V_{NAR}, engineered VH or VK domains. VHHs can be generated from camelid heavy chain only antibodies. V_{NARS} can be generated from cartilaginous fish heavy chain only antibodies. Various methods have been implemented to generate monomeric sdAbs from conventionally heterodimeric VH and VK domains, including interface engineering and selection of specific germline families. In other embodiments, the isolated polypeptides are derived from non-antibody scaffold proteins for example but not limited to designed ankyrin repeat proteins (darpins), avimers, anticalin/lipocalins, centyrins and fynomers.

In some embodiments, the isolated polypeptide includes a first amino acid sequence that binds 4B11 selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83, and a second amino acid sequence that binds PDL1 selected from the group consisting of SEQ ID NO: 100, 104, 108, 112, 114, 116, and 119-124.

In some embodiments, the isolated polypeptide includes a first amino acid sequence that binds 4B11 selected from the group consisting of SEQ ID NO: 33, 39, 33-41, 43, 45-47, 49, 51, 53, 54, 56, 58-60, 62, 65, 66, 68, 70, 72, 74, 76, 78, and 80-83, and a second amino acid sequence that binds PDL1 selected from the group consisting of SEQ ID NO: 119-124.

In some embodiments, the isolated polypeptide includes a first amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence that binds 4B111 selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83, and a second amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence that binds PDL1 selected from the group consisting of SEQ ID NO: 100, 104, 108, 112, 114, 116, and 119-124.

In some embodiments, the isolated polypeptide includes a first amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence that binds 4B111 selected from the group consisting of SEQ ID NO: 33, 39, 33-41, 43, 45-47, 49, 51, 53, 54, 56, 58-60, 62, 65, 66, 68, 70, 72, 74, 76, 78, and 80-83, and a second amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence that binds PDL1 selected from the group consisting of SEQ ID NO: 119-124.

In some embodiments, the isolated polypeptide includes (i) a first amino acid sequence that binds 4B11 and comprises a complementarity determining region 1 (CDRT) comprising an amino acid sequence selected from the group consisting of SEQ ID NO: 17, 21, 26, 30, 50, 65, and 69; a complementarity determining region 2 (CDR2) comprising an amino acid sequence selected from the group consisting of SEQ ID NO: 18, 27, 31, 42, 44, 48, 52, 61, 63, 71, 73, 75, 77, and 79; and a complementarity determining region 3 (CDR3) comprising an amino acid sequence selected from the group consisting of SEQ ID NO: 19, 22, 24, 28, 32, 55, and 57; and (ii) a second amino acid sequence that binds PDL1 and comprises a CDR1 comprising an amino acid sequence selected from the group consisting of SEQ ID NO: 101, 105, and 109; a CDR2 comprising an amino acid sequence selected from the group consisting of SEQ ID NO: 102, 106, 110, and 117; and a CDR3 comprising an amino acid sequence selected from the group consisting of SEQ ID NO: 103, 107, 111, 113, 115, and 118.

In some embodiments, the binding domains (BDs) of the present disclosure, e.g., the 41BB-binding domains and/or the PDL1-binding domains, are derived from antibodies or antibody fragments including scFv, Fabs, single domain antibodies (sdAb), V_{NAR}, or VHHs. In some embodiments, the BDs are human or humanized sdAb. The sdAb fragments, can be derived from VHH, V_{NAR}, engineered VH or VK domains. VHHs can be generated from camelid heavy chain only antibodies. V_{NARS} can be generated from cartilaginous fish heavy chain only antibodies. Various methods have been implemented to generate monomeric sdAbs from conventionally heterodimeric VH and VK domains, including interface engineering and selection of specific germline families. In other embodiments, the BDs are derived from non-antibody scaffold proteins for example but not limited to designed ankyrin repeat proteins (darpins), avimer, anticalin/lipocalins, centyrins and fynomers.

Generally, the fusion proteins of the present disclosure consist of at least two or more BDs operably linked via a linker polypeptide. The utilization of sdAb fragments as the specific BD within the fusion the present disclosure has the benefit of avoiding the heavy chain: light chain mis-pairing problem common to many bi/multispecific antibody approaches. In addition, the fusion proteins of the present disclosure avoid the use of long linkers necessitated by many bispecific antibodies.

In some embodiments, all of the BDs of the fusion protein are TBDs that recognize the same epitope on the given TNFRSF member. For example, the fusion proteins of present disclosure may incorporate 2, 3, 4, 5, or 6 TBDs with identical specificity to 41BB. In other embodiments, the fusion protein incorporates TBDs that recognize distinct epitopes on the given TNFRSF member. For example, the fusion proteins of present disclosure may incorporate 2, 3, 4, 5, or 6 TBDs with distinct recognition specificities toward various epitopes on 41BB. In these embodiments, the fusion proteins of the present disclosure contain multiple TBDs that target distinct regions of the particular TNFRSF member. In some embodiments, the TBDs may recognize different epitopes on the same TNFRSF member or recognize epitopes on distinct TNFRSF members. For example, the present disclosure provides multispecific fusion proteins incorporating TBDs that bind GITR and 41BB or OX40 and 41BB, or CD27 and 41BB.

In some embodiments, the multispecific fusion protein is a bispecific molecule that targets 41BB and PDL1. In some embodiments, the bispecific fusion protein includes a 41BB-targeting binding domain selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83, operably linked to a second binding domain (BD2) that binds PDL1. In some embodiments, the BD2 comprises an amino acid sequence that specifically binds PDL1. In some embodiments, the BD2 comprises a PDL1-targeting domain selected from the group consisting of SEQ ID NO: 100, 104, 108, 112, 114, 116, and 119-124. some embodiments, the BD2 comprises a PDL1-targeting domain selected from the group consisting of SEQ ID NO: 119-124. In some embodiments, the BD2 comprises an amino acid sequence that specifically binds PDL1 and is selected from the group consisting of SEQ ID NO: 126-408.

In some embodiments, the multispecific fusion protein is a bispecific molecule that targets 41BB and PDL1. In some embodiments, the bispecific fusion protein includes a 41BB-targeting binding domain selected from the group consisting of SEQ ID NO: 33-41, 43, 45-47, 49, 51, 53, 54, 56, 58-60, 62, 65, 66, 68, 70, 72, 74, 76, 78, and 80-83, operably linked to a second binding domain (BD2) that binds PDL1. In some embodiments, the BD2 comprises an amino acid sequence that specifically binds PDL1. In some embodiments, the BD2 comprises a PDL1-targeting domain selected from the group consisting of SEQ ID NO: 100, 104, 108, 112, 114, 116, and 119-124. In some embodiments, the BD2 comprises a PDL1-targeting domain selected from the group consisting of SEQ ID NO: 119-124. In some embodiments, the BD2 comprises an amino acid sequence that specifically binds PDL1 and is selected from the group consisting of SEQ ID NO: 126-408.

In some embodiments, the multispecific fusion protein is a bispecific molecule that targets 41BB and PDL1. In some embodiments, the bispecific fusion protein includes a PDL1-targeting binding domain selected from the group consisting of SEQ ID NO: 100, 104, 108, 112, 114, 116, and 119-124, operably linked to a second TBD (TBD2) that binds 41BB. In some embodiments, the TBD2 comprises an amino acid sequence that specifically binds 41BB. In some embodiments, the TBD2 comprises a 41BB-targeting domain selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83. In some embodiments, the TBD2 comprises an amino acid sequence that specifically binds 41BB and is selected from the group consisting of SEQ ID NO: 84-99.

In some embodiments, the multispecific fusion protein is a bispecific molecule that targets 41BB and PDL1. In some embodiments, the bispecific fusion protein includes a PDL1-targeting binding domain selected from the group consisting of SEQ ID NO: 119-124, operably linked to a second TBD (TBD2) that binds 41BB. In some embodiments, the TBD2 comprises an amino acid sequence that specifically binds 41BB. In some embodiments, the TBD2 comprises a 41BB-targeting domain selected from the group consisting of SEQ ID NO: 33, 39, 33-41, 43, 45-47, 49, 51, 53, 54, 56, 58-60, 62, 65, 66, 68, 70, 72, 74, 76, 78, and 80-83. In some embodiments, the TBD2 comprises an amino acid sequence that specifically binds 41BB and is selected from the group consisting of SEQ ID NO: 84-99.

In some embodiments, the multispecific fusion protein is a bispecific molecule that targets 41BB and PDL1 and comprises an amino acid sequence that is selected from the group consisting of SEQ ID NO: 448-456.

In some embodiments, the multispecific fusion protein is a bispecific molecule that targets 41BB and PDL1 and comprises an amino acid sequence that is at least 50%, 60%, 65%, 70%, 75%, 80%, 85%, 90%, 91%, 92%, 93%, 94%, 95%, 96%, 97%, 98%, or 99% identical to an amino acid sequence selected from the group consisting of SEQ ID NO: 448-456.

In some embodiments, all of the BDs of the fusion protein recognize the same epitope on PDL1. For example, the fusion proteins of present disclosure may incorporate 2, 3, 4, 5, or 6 BDs with identical specificity to PDL1. In other embodiments, the fusion protein incorporates BDs that recognize distinct epitopes on PDL1. For example, the fusion proteins of present disclosure may incorporate 2, 3, 4, 5, or 6 BDs with distinct recognition specificities toward various epitopes on PDL1. In these embodiments, the fusion proteins of the present disclosure contain multiple BDs that target distinct regions of the PDL1. In some embodiments, the BDs may recognize different epitopes on PDL1.

In some embodiments, the fusion protein of the present disclosure is composed of a single polypeptide. In other embodiments, the fusion protein of the present disclosure is composed of more than one polypeptide. For example, wherein a heterodimerization domain is incorporated into the fusion protein so as the construct an asymmetric fusion protein. For example, if an immunoglobulin Fc region is incorporated into the fusion protein the CH3 domain can be used as a homodimerization domain, or the CH3 dimer interface region can be mutated so as to enable heterodimerization.

In some embodiments, the fusion protein contains the BDs opposite ends. For example, the BDs are located on both the amino-terminal (N-terminal) portion of the fusion protein and the carboxy-terminal (C-terminal) portion of the fusion protein. In other embodiments, all the TBDs reside on the same end of the fusion protein. For example, BDs reside on either the amino- or carboxy-terminal portions of the fusion protein.

In some embodiments, the linker polypeptide contains an immunoglobulin Fc region. In some embodiments, the immunoglobulin Fc region is an IgG isotype selected from the group consisting of IgG1 subclass, IgG2 subclass, IgG3 subclass, and IgG4 subclass.

In some embodiments, the immunoglobulin Fc region or immunologically active fragment thereof is an IgG isotype. For example, the immunoglobulin Fc region of the fusion protein is of human IgG1 subclass, having an amino acid sequence:

| (SEQ ID NO: 1) | | | | |
|---|---|---|---|---|
| 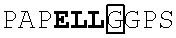 | VFLFPPKPKD | TLMISRTPEV | TCVVVDVSHE | DPEVKFNWYV |
| DGVEVHNAKT | 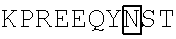 | YRVVSVLTVL | HQDWLNGKEY | KCKVSNKALP |
| APIEKTISKA | KGQPREPQVY | TLPPSRDELT | KNQVSLTCLV | KGFYPSDIAV |
| EWESNGQPEN | NYKTTPPVLD | SDGSFFLYSK | LTVDKSRWQQ | GNVFSCSVMH |
| EALHNHYTQK | SLSLSPGK | | | |

In some embodiments, the immunoglobulin Fc region or immunologically active fragment thereof comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 1.

In some embodiments, the human IgG1 Fc region is modified at amino acid Asn297 (Boxed in SEQ ID NOs: 1-4, Kabat Numbering) to prevent to glycosylation of the fusion protein, e.g., Asn297Ala (N297A) or Asn297Asp (N297D). In some embodiments, the Fc region of the fusion protein is modified at amino acid Leu235 (Bold in SEQ ID NO: 1, Kabat Numbering) to alter Fc receptor interactions, e.g., Leu235Glu (L235E) or Leu235Ala (L235A). In some embodiments, the Fc region of the fusion protein is modified at amino acid Leu234 (Bold in SEQ ID NO: 1, Kabat Numbering) to alter Fc receptor interactions, e.g., Leu234Ala (L234A). In some embodiments, the Fc region of the fusion protein is modified at amino acid Leu234 (Boxed, Kabat Numbering) to alter Fc receptor interactions, e.g., Leu235Glu (L235E). In some embodiments, the Fc region of the fusion protein is altered at both amino acid 234 and 235, e.g., Leu234Ala and Leu235Ala (L234A/L235A) or Leu234Val and Leu235Ala (L234V/L235A). In some embodiments, the Fc region of the fusion protein is lacking an amino acid at one or more of the following positions to reduce Fc receptor binding: Glu233 (E233, Bold in SEQ ID NO: 1), Leu234 (L234), or Leu235 (L235). In some embodiments, the Fc region of the fusion protein is altered at Gly235 to reduce Fc receptor binding. For example, wherein Gly235 is deleted from the fusion protein. In some embodiments, the human IgG1 Fc region is modified at amino acid Gly236 (Boxed in SEQ ID NO: 1) to enhance the interaction with CD32A, e.g., Gly236Ala (G236A). In some embodiments, the human IgG1 Fc region lacks Lys447 (EU index ofKabat et al 1991 Sequences ofProteins oflmmunologicalInterest).

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) orAla327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) orAla327Ser (A327S). In preferred embodiments, modifications within the Fc region reduce binding to Fc-receptor-gamma receptors while have minimal impact on binding to the neonatal Fc receptor (FcRn).

In some embodiments, the Fc region of the fusion protein is lacking an amino acid at one or more of the following positions to reduce Fc receptor binding: Glu233 (E233), Leu234 (L234), or Leu235 (L235). In these embodiments, Fc deletion of these three amino acids reduces the complement protein C1q binding. These modified Fc region polypeptides are referred to herein as “Fc deletion” polypeptides.

(SEQ ID NO: 2)
PAPGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSRDELTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

In some embodiments, the immunoglobulin Fc region or immunologically active fragment thereof comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 2.

In some embodiments, the immunoglobulin Fc region or immunologically active fragment of the fusion protein is of human IgG2 subclass, having an amino acid sequence:

| (SEQ ID NO: 3) | | | | |
|---|---|---|---|---|
| PAPPVAGPSV | FLFPPKPKDT | LMISRTPEVT | CVVVDVSHED | PEVQFNWYVD |
| GVEVHNAKTK | 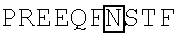 | RVVSVLTVVH | QDWLNGKEYK | CKVSNKGLPA |
| PIEKTISKTK | GQPREPQVYT | LPPSREEMTK | NQVSLTCLVK | GFYPSDISVE |
| WESNGQPENN | YKTTPPMLDS | DGSFFLYSKL | TVDKSRWQQG | NVFSCSVMHE |
| ALHNHYTQKS | LSLSPGK | | | |

In some embodiments, the fusion or immunologically active fragment thereof comprises a human IgG2 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 3.

In some embodiments, the human IgG2 Fc region is modified at amino acid Asn297 (Boxed in SEQ ID NOs: 1, 3, 4, and 5), to prevent to glycosylation of the antibody, e.g., Asn297Ala (N297A). In some embodiments, the human IgG2 Fc region lacks Lys447, which corresponds to residue 217 of SEQ ID NO: 3 (EU index ofKabat et al 1991 Sequences of Proteins oflmmunologicalInterest).

In some embodiments, the immunoglobulin Fc region or immunologically active fragment of the fusion protein is of human IgG3 subclass, having an amino acid sequence:

| (SEQ ID NO: 4) | | | | |
|---|---|---|---|---|
| PAPELLGGPS | VFLFPPKPKD | TLMISRTPEV | TCVVVDVSHE | DPEVQFKWYV |
| DGVEVHNAKT | 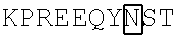 | FRVVSVLTVL | HQDWLNGKEY | KCKVSNKALP |
| APIEKTISKT | KGQPREPQVY | TLPPSREEMT | KNQVSLTCLV | KGFYPSDIAV |
| EWESSGQPEN | NYNTTPPMLD | SDGSFFLYSK | LTVDKSRWQQ | GNIFSCSVMH |
| 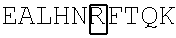 | SLSLSPGK | | | |

In some embodiments, the antibody or immunologically active fragment thereof comprises a human IgG3 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 4.

In some embodiments, the human IgG3 Fc region is modified at amino acid Asn297 (Boxed in SEQ ID NOs: 1-4, Kabat Numbering) to prevent to glycosylation of the antibody, e.g., Asn297Ala (N297A). In some embodiments, the human IgG3 Fc region is modified at amino acid 435 to extend the half-life, e.g., Arg435His (R435H, Boxed in SEQ ID NO: 3). In some embodiments, the human IgG3 Fc region lacks Lys447, which corresponds to residue 218 of SEQ ID NO: 4 (EU index of Kabat et al 1991 Sequences ofProteins of Immunological Interest).

In some embodiments, the immunoglobulin Fc region or immunologically active fragment of the fusion protein is of human IgG4 subclass, having an amino acid sequence:

| (SEQ ID NO: 5) | | | | |
|---|---|---|---|---|
| 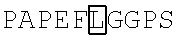 | VFLFPPKPKD | TLMISRTPEV | TCVVVDVSQE | DPEVQFNWYV |
| DGVEVHNAKT | 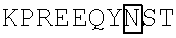 | YRVVSVLTVL | HQDWLNGKEY | KCKVSNKGLP |
| SSIEKTISKA | KGQPREPQVY | TLPPSQEEMT | KNQVSLTCLV | KGFYPSDIAV |
| EWESNGOPEN | NYKTTPPVLD | SDGSFFLYSR | LTVDKSRWQE | GNVESCSVMH |
| EALHNHYTQK | SLSLSLGK | | | |

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 5.

In other embodiments, the human IgG4 Fc region is modified at amino acid 235 to alter Fc receptor interactions, e.g., Leu235Glu (L235E). In some embodiments, the human IgG4 Fc region is modified at amino acid Asn297 (Boxed in SEQ ID NOs: 1-4, Kabat Numbering) to prevent to glycosylation of the antibody, e.g., Asn297Ala (N297A). In some embodiments, the human IgG4 Fc region lacks Lys447, which corresponds to residue 218 of SEQ ID NO: 5 (EU index ofKabat et al 1991 Sequences ofProteins of lmmunological Interest).

In some embodiments, the immunoglobulin Fc region or immunologically active fragment of the fusion protein is of human IgG4 isotype, having an amino acid sequence:

| (SEQ ID NO: 6) | | | | |
|---|---|---|---|---|
| PAPELLGGPS | VFLFPPKPKD | TLMISRTPEV | TCVVVDVSQE | DPEVQFNWYV |
| DGVEVHNAKT | 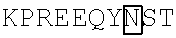 | YRVVSVLTVL | HQDWLNGKEY | KCKVSNKGLP |
| SSIEKTISKA | KGQPREPQVY | TLPPSQEEMT | KNQVSLTCLV | KGFYPSDIAV |
| EWESNGQPEN | NYKTTPPVLD | SDGSFFLYSR | LTVDKSRWQE | GNVFSCSVMH |
| EALHNHYTQK | SLSLSLGK | | | |

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 6.

In some embodiments, the human IgG Fc region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281(33) 23514-23524), Met428Leu and Asn434Ser (M428L, N434S) (Zalevsky et al 2010 Nature Biotech, Vol. 28(2) 157-159), or Met252Ile, Thr256Asp, Met428Leu (M252I, T256D, M428L, respectively), (EU index of Kabat et al 1991 Sequences ofProteins of Immunological Interest). Met252 corresponds to residue 23 in SEQ ID NOs: 1, 4, and 5 and residue 22 in SEQ ID NO: 3. Ser254 corresponds to corresponds to residue 25 in SEQ ID NOs: 1, 4, and 5 and residue 24 in SEQ ID NO: 3. Thr256 corresponds to residue 27 in SEQ ID NOs: 1, 4, and 5 and residue 26 in SEQ ID NO: 3. Met428 corresponds to residue 199 in SEQ ID NOs: 1, 4, and 5 and residue 198 in SEQ ID NO: 3. Asn434 corresponds to residue 205 in SEQ ID NOs: 1, 4, and 5 and residue 204 in SEQ ID NO: 3. In some embodiments where the fusion protein of the disclosure includes an Fc polypeptide, the Fc polypeptide is mutated or modified. In these embodiments, the mutated or modified Fc polypeptide includes the following mutations: Met252Tyr and Met428Leu (M252Y, M428L) using the Kabat numbering system.

In some embodiments, the human IgG Fc region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11. Examples of mutations that enhance ADCC include modification at Ser239 and I1e332, for example Ser239Asp and Ile332Glu (S239D, 1332E). Examples of mutations that enhance CDC include modifications at Lys326, which corresponds to residue 97 of SEQ ID NOs: 1, 4, and 5 and residue 96 of SEQ ID NO: 2, and Glu333, which corresponds to residue 104 of SEQ ID NOs: 1, 4, and 5 and residue 103 of SEQ ID NO: 3. In some embodiments the Fc region is modified at one or both of these positions, for example Lys326Ala and/or Glu333Ala (K326A and E333A).

In some embodiments, the human IgG Fc region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, which corresponds to residue 137 of SEQ ID NOs: 1, 4, and 5 and residue 136 of SEQ ID NO: 3, Leu368, which corresponds to residue 139 of SEQ ID NOs: 1, 4, and 5 and residue 138 of SEQ ID NO: 2, and Tyr407, which corresponds to residue 178 of SEQ ID NOs: 1, 4, and 5 and residue 177 of SEQ ID NO: 3, e.g., Ser, Ala and Val, respectively (T366S/L368A/Y407V). Heterodimerization via CH3 modifications can be further stabilized by the introduction of a disulfide bond, for example by changing Ser354, which corresponds to residue 125 of SEQ ID NOs: 1, 4, and 5 and residue 124 of SEQ ID NO: 3, to Cys (S354C) and Tyr349, which corresponds to residue 120 of SEQ ID NOs: 1, 4, and 5 and residue 119 of SEQ ID NO: 3, to Cys (Y349C) on opposite CH3 domains (Reviewed in Carter, 2001 Journal of Immunological Methods, 248: 7-15). In some of these embodiments, the Fc region may be modified at the protein-A binding site on one member of the heterodimer so as to prevent protein-A binding and thereby enable more efficient purification of the heterodimeric fusion protein. An exemplary modification within this binding site is I1e253, which corresponds to residue 24 of SEQ ID NOs: 1, 4, and 5 and residue 23 of SEQ ID NO: 3, for example Ile253Arg (1253R). For example, the 1253R modification may be combined with either the T366S/L368A/Y407V modifications or with the T366W modifications. The T366S/L368A/Y407V modified Fc is capable of forming homodimers as there is no steric occlusion of the dimerization interface as there is in the case of the T336W modified Fc. Therefore, in some embodiments, the I253R modification is combined with the T366S/L368A/Y407V modified Fc to disallow purification any homodimeric Fc that may have formed.

In some embodiments, the human IgG Fc region is modified to prevent dimerization. In these embodiments, the fusion proteins of the present disclosure are monomeric. For example, modification at residue Thr366 to a charged residue, e.g. Thr366Lys, Thr366Arg, Thr366Asp, or Thr366Glu (T366K, T366R, T366D, or T366E, respectively), prevents CH3-CH3 dimerization.

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) orAla327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) orAla327Ser (A327S). In preferred embodiments, modifications within the Fc region reduce binding to Fc-receptor-gamma receptors while have minimal impact on binding to the neonatal Fc receptor (FcRn).

In some embodiments, the fusion protein contains a polypeptide derived from an immunoglobulin hinge region. The hinge region can be selected from any of the human IgG subclasses. For example, the fusion protein may contain a modified IgG1 hinge having the sequence of EPKSSDKTHTCPPC (SEQ ID NO: 7), where in the Cys220 that forms a disulfide with the C-terminal cysteine of the light chain is mutated to serine, e.g., Cys220Ser (C220S). In other embodiments, the fusion protein contains a truncated hinge having a sequence DKTHTCPPC (SEQ ID NO: 8).

In some embodiments, the fusion protein has a modified hinge from IgG4, which is modified to prevent or reduce strand exchange, e.g., Ser228Pro (S228P), having the sequence ESKYGPPCPPC (SEQ ID NO: 9). In some embodiments, the fusion protein contains one or more linker polypeptides. In other embodiments, the fusion protein contains linker and hinge polypeptides.

In some embodiments, the fusion proteins of the present disclosure lack or have reduced Fucose attached to the N-linked glycan-chain at N297. There are numerous ways to prevent fucosylation, including but not limited to production in a FUT8 deficient cell line; addition inhibitors to the mammalian cell culture media, for example Castanospermine, 2-deoxy-fucose, 2-flurofucose; the use of production cell lines with naturally reduced fucosylation pathways and metabolic engineering of the production cell line.

In some embodiments, the single domain antibody, VHH, or humanized single domain antibody, or human single domain antibody is engineered to eliminate recognition by pre-existing antibodies found in humans. In some embodiments, single domain antibodies of the present disclosure are modified by mutation of position Leul1, for example LeullGlu (L11E) or Leul1Lys (L 11K). In other embodiments, single domain antibodies of the present disclosure are modified by changes in carboxy-terminal region, for example the terminal sequence consists of GQGTLVTVKPGG (SEQ ID NO: 14) or GQGTLVTVEPGG (SEQ ID NO: 15) or modification thereof. In some embodiments, the single domain antibodies of the present disclosure are modified by mutation of position 11 and by changes in carboxy-terminal region.

In some embodiments, the BDs of the fusion proteins of the present disclosure are operably linked via amino acid linkers. In some embodiments, these linkers are composed predominately of the amino acids Glycine and Serine, denoted as GS-linkers herein. The GS-linkers of the fusion proteins of the present disclosure can be of various lengths, for example 5, 6, 7, 8, 9, 10, 11, 12, 13, 14, 15, 16, 17, 18, 19, 20 amino acids in length.

In some embodiments, the GS-linker comprises an amino acid sequence selected from the group consisting of GGSGGS, i.e., (GGS)₂ (SEQ ID NO: 10); GGSGGSGGS, i.e., (GGS)₃ (SEQ ID NO: 11); GGSGGSGGSGGS, i.e., (GGS)₄ (SEQ ID NO: 12); and GGSGGSGGSGGSGGS, i.e., (GGS)₅ (SEQ ID NO: 13).

In some embodiments, the multivalent binding fusion protein is tetravalent. In some embodiments, the tetravalent fusion protein has the following structure: BD-Linker-BD-Linker-Hinge-Fc. In some embodiments, the tetravalent fusion protein has the following structure: BD-Linker-Hinge-Fc-Linker-BD.

In some embodiments, the BD of the tetravalent fusion protein is a single domain antibody or VHH. In some embodiments, each BD of the tetravalent fusion protein is a single domain antibody or VHH. In some embodiments, the tetravalent fusion protein has the following structure: VHH-Linker-VHH-Linker-Hinge-Fc, where the VHH is a humanized or fully human VHH sequence. In some embodiments, the tetravalent fusion protein has the following structure: VHH-Linker-Hinge-Fc-Linker-VHH, where the VHH is a humanized or fully human VHH sequence.

In some embodiments, the multivalent TNFRSF binding fusion protein is tetravalent. In some embodiments, the tetravalent TNFRSF binding fusion protein has the following structure: TBD-Linker-TBD-Linker-Hinge-Fc. In some embodiments, the tetravalent TNFRSF binding fusion protein has the following structure: TBD-Linker-Hinge-Fc-Linker-TBD.

In some embodiments, the TBD of the tetravalent TNFRSF binding fusion protein is a single domain antibody or VHH. In some embodiments, each TBD of the multivalent TNFRSF binding fusion protein is single domain antibody or VHH. In some embodiments, the tetravalent TNFRSF binding fusion protein has the following structure: VHH-Linker-VHH-Linker-Hinge-Fc, where the VHH is a humanized or fully human VHH sequence. In some embodiments, the tetravalent TNFRSF binding fusion protein has the following structure: VHH-Linker-Hinge-Fc-Linker-VHH, where the VHH is a humanized or fully human VHH sequence.

In some embodiments, the GS-linker comprises an amino acid sequence selected from the group consisting of GGSGGS, i.e., (GGS)₂ (SEQ ID NO: 10); GGSGGSGGS, i.e., (GGS)₃ (SEQ ID NO: 11); GGSGGSGGSGGS, i.e., (GGS)₄ (SEQ ID NO: 12); and GGSGGSGGSGGSGGS, i.e., (GGS)₅ (SEQ ID NO: 13).

In some embodiments, the multivalent fusion protein is hexavalent. In some embodiments, the hexavalent fusion protein has the following structure: BD-Linker-TBD-Linker-BD-Linker-Hinge-Fc. In some embodiments, the hexavalent fusion protein has the following structure: BD-Linker-BD-Linker-Hinge-Fc-Linker-BD, or BD-Linker-Hinge-Fc-Linker-BD-Linker-BD.

In some embodiments, the BD of the hexavalent fusion protein is a single domain antibody or VHH. In some embodiments, each BD of the hexavalent fusion protein is a single domain antibody or VHH. In some embodiments, the hexavalent fusion protein has the following structure: VHH-Linker-VHH-Linker-VHH-Linker-Hinge-Fc, where the VHH is a humanized or fully human VHH sequence. In some embodiments, the hexavalent fusion protein has the following structure: VHH-Linker-VHH-Linker-Hinge-Fc-Linker-VHH, or VHH-Linker-Hinge-Fc-Linker-VHH-Linker-VHH where the VHH is a humanized or fully human VHH sequence.

In some embodiments, the multivalent TNFRSF binding fusion protein is hexavalent. In some embodiments, the hexavalent TNFRSF binding fusion protein has the following structure: TBD-Linker-TBD-Linker-TBD-Linker-Hinge-Fc. In some embodiments, the hexavalent TNFRSF binding fusion protein has the following structure: TBD-Linker-TBD-Linker-Hinge-Fc-Linker-TBD, or TBD-Linker-Hinge-Fc-Linker-TBD-Linker-TBD.

In some embodiments, the TBD of the hexavalent TNFRSF binding fusion protein is a single domain antibody or VHH. In some embodiments, each TBD of the hexavalent TNFRSF binding fusion protein is a single domain antibody or VHH. In some embodiments, the hexavalent TNFRSF binding fusion protein has the following structure: VHH-Linker-VHH-Linker-VHH-Linker-Hinge-Fc, where the VHH is a humanized or fully human VHH sequence. In some embodiments, the hexavalent TNFRSF binding fusion protein has the following structure: VHH-Linker-VHH-Linker-Hinge-Fc-Linker-VHH, or VHH-Linker-Hinge-Fc-Linker-VHH-Linker-VHH where the VHH is a humanized or fully human VHH sequence.

In some embodiments, the multivalent fusion protein lacks an Fc region. In some of these embodiments, the fusion protein is tetravalent and has the following structure BD-Linker-BD-Linker-BD-Linker-BD-Linker. In some of these embodiments, the fusion protein is pentavalent and has the following structure BD-Linker-BD-Linker-BD-Linker-BD-Linker-BD. In some of these embodiments, the fusion protein is hexavalent and has the following structure BD-Linker-BD-Linker-BD-Linker-BD-Linker-BD-Linker-BD.

In some embodiments, the multivalent TNFRSF binding fusion protein lacks an Fc region. In some of these embodiments, the TNFRSF binding fusion protein is tetravalent and has the following structure TBD-Linker-TBD-Linker-TBD-Linker-TBD-Linker. In some of these embodiments, the TNFRSF binding fusion protein is pentavalent and has the following structure TBD-Linker-TBD-Linker-TBD-Linker-TBD-Linker-TBD. In some of these embodiments, the TNFRSF binding fusion protein is hexavalent and has the following structure TBD-Linker-TBD-Linker-TBD-Linker-TBD-Linker-TBD-Linker-TBD.

In some embodiments, the BD of a multivalent fusion protein is a single domain antibody or VHH. In some embodiments, the multivalent fusion protein lacks an Fc region. In some of these embodiments, the fusion protein is tetravalent and has the following structure VHH-Linker-VHH-Linker-VHH-Linker-VHH-Linker. In some of these embodiments, the fusion protein is pentavalent and has the following structure VHH-Linker-VHH-Linker-VHH-Linker-VHH-Linker-VHH. In some of these embodiments, the fusion protein is hexavalent and has the following structure VHH-Linker-VHH-Linker-VHH-Linker-VHH-Linker-VHH-Linker-VHH. In any of these embodiments, the VHH is a humanized or fully human VHH sequence.

In some embodiments, the TBD of the a multivalent TNFRSF binding fusion protein is a single domain antibody or VHH. In some embodiments, the multivalent TNFRSF binding fusion protein lacks an Fc region. In some of these embodiments, the TNFRSF binding fusion protein is tetravalent and has the following structure VHH-Linker-VHH-Linker-VHH-Linker-VHH-Linker. In some of these embodiments, the TNFRSF binding fusion protein is pentavalent and has the following structure VHH-Linker-VHH-Linker-VHH-Linker-VHH-Linker-VHH. In some of these embodiments, the TNFRSF binding fusion protein is hexavalent and has the following structure VHH-Linker-VHH-Linker-VHH-Linker-VHH-Linker-VHH-Linker-VHH. In any of these embodiments, the VHH is a humanized or fully human VHH sequence.

In some embodiments, the GS-linker comprises an amino acid sequence selected from the group consisting of GGSGGS, i.e., (GGS)₂ (SEQ ID NO: 10); GGSGGSGGS, i.e., (GGS)₃ (SEQ ID NO: 11); GGSGGSGGSGGS, i.e., (GGS)₄ (SEQ ID NO: 12); and GGSGGSGGSGGSGGS, i.e., (GGS)₅ (SEQ ID NO: 13).

In some embodiments, the fusion proteins are multispecific containing a TBD and a binding domain directed toward a second antigen. In these embodiments, the second antigen binding domain can be positioned at numerous positions within the molecule relative to the TBD. In some embodiments, the second antigen binding domain is located N-terminal TBD. In other embodiments, the second antigen binding domain is located to C-terminal to the TBD. In other embodiments, the second antigen binding domain is located on a distinct polypeptide that associates with a first polypeptide containing the TBD.

In some embodiments, the fusion proteins are multispecific containing an anti-41BB binding domain and a binding domain directed toward a second antigen. In these embodiments, the second antigen binding domain can be positioned at numerous positions within the molecule relative to the an anti-41BB binding domain. In some embodiments, the second antigen binding domain is located N-terminal an anti-41BB binding domain. In other embodiments, the second antigen binding domain is located to C-terminal to the an anti-41BB binding domain. In other embodiments, the second antigen binding domain is located on a distinct polypeptide that associates with a first polypeptide containing the an anti-41BB binding domain.

In some embodiments, the fusion proteins are multispecific containing an anti-PDL1 binding domain and a binding domain directed toward a second antigen. In these embodiments, the second antigen binding domain can be positioned at numerous positions within the molecule relative to the an anti-PDL1 binding domain. In some embodiments, the second antigen binding domain is located N-terminal an anti-PDL1 binding domain. In other embodiments, the second antigen binding domain is located to C-terminal to the an anti-PDL1 binding domain. In other embodiments, the second antigen binding domain is located on a distinct polypeptide that associates with a first polypeptide containing the an anti-PDL1 binding domain.

In some embodiments, the TBD within the multispecific TNFRSF binding fusion protein is a single domain antibody or VHH. In some embodiments, the TBD within the multispecific TNFRSF binding fusion protein is a composed of antibody variable heavy (VH) chain and variable light (VL) chain region. In some embodiments, the VH and VL of the TBD are formatted as a single chain variable fragment (scFv) connected via a linker region. In some embodiments, the VH and VL of the TBD are formatted as a FAB fragment that associates via a constant heavy 1 (CH1) domain and a constant light chain (CL) domain. In some embodiments, non-antibody heterodimerization domains are utilized to enable the proper association of the VH and VL of the TBD. In some embodiments, the TBD within the multispecific TNFRSF binding fusion protein is derived from non-antibody scaffold proteins for example but not limited to designed ankyrin repeat proteins (darpins), avimer, anticalin/lipocalins, centyrins and fynomers.

In some embodiments, the TBD within the multispecific TNFRSF binding fusion protein is a single domain antibody or VHH that binds 41BB. In some embodiments, the anti-41BB binding domain within the multispecific TNFRSF binding fusion protein is a composed of antibody variable heavy (VH) chain and variable light (VL) chain region. In some embodiments, the VH and VL of the anti-41BB binding domain are formatted as a single chain variable fragment (scFv) connected via a linker region. In some embodiments, the VH and VL of the anti-41BB binding domain are formatted as a Fab fragment that associates via a constant heavy 1 (CH1) domain and a constant light chain (CL) domain. In some embodiments, non-antibody heterodimerization domains are utilized to enable the proper association of the VH and VL of the anti-41BB binding domain. In some embodiments, the anti-41BB binding domain within the multispecific TNFRSF binding fusion protein is derived from non-antibody scaffold proteins for example but not limited to designed ankyrin repeat proteins (darpins), avimer, anticalin/lipocalins, centyrins and fynomers.

In some embodiments, the binding domain within the multispecific fusion protein is a single domain antibody or VHH that binds PDL1. In some embodiments, the anti-PDL1 binding domain within the multispecific TNFRSF binding fusion protein is a composed of antibody variable heavy (VH) chain and variable light (VL) chain region. In some embodiments, the VH and VL of the anti-PDL1 binding domain are formatted as a single chain variable fragment (scFv) connected via a linker region. In some embodiments, the VH and VL of the anti-PDL1 binding domain are formatted as a Fab fragment that associates via a constant heavy 1 (CH1) domain and a constant light chain (CL) domain. In some embodiments, non-antibody heterodimerization domains are utilized to enable the proper association of the VH and VL of the anti-PDL1 binding domain. In some embodiments, the anti-PDL1 binding domain within the multispecific fusion protein is derived from non-antibody scaffold proteins for example but not limited to designed ankyrin repeat proteins (darpins), avimer, anticalin/lipocalins, centyrins and fynomers.

In some embodiments, the anti-41BB binding domain of the multispecific TNFRSF binding fusion protein is a bispecific antibody or antigen-binding fragment thereof.

In some embodiments, the anti-PDL1 binding domain of the multispecific fusion protein is a bispecific antibody or antigen-binding fragment thereof.

In any of these embodiments, the bispecific antibody or antigen-fragment thereof can be any suitable bispecific format known in the art, including, by way of non-limiting example, formats based on antibody fragments such as, e.g., X-Link Fab, cross-linked Fab fragments; tascFv/BiTE, tandem-scFv/Bispecific T cell Engager; Db, diabody; taDb, tandem diabody; formats based on Fc-fusions such as, e.g., Db-Fc, diabody-Fc fusion; taDb-Fc fusion, tandem diabody-Fc fusion; taDb-CH3, tandem diabody-CH3 fusion; (scFv)₄-Fc, tetra scFv-Fc fusion; DVD-Ig, dual variable domain immunoglobulin; IgG formats such as, e.g., knob-hole and SEED, strand exchange engineered domain; CrossMab, knob-hole combined with heavy and light chain domain exchange; bsAb, quadroma derived bispecific antibody; sdAb, single domain based antibody; and kappa-lambda bodies such as those described in PCT Publication No. WO 2012/023053.

In any of the above embodiments, at least one TBD comprises an amino acid sequence selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83.

In any of the above embodiments, at least one TBD comprises an amino acid sequence selected from the group consisting of SEQ ID NO: 33, 39, 33-41, 43, 45-47, 49, 51, 53, 54, 56, 58-60, 62, 65, 66, 68, 70, 72, 74, 76, 78, and 80-83.

In any of the above embodiments, at least one TBD comprises a complementarity determining region 1 (CDR1) comprising an amino acid sequence selected from the group consisting of SEQ ID NO: 17, 21, 26, 30, 50, 65, and 69; a complementarity determining region 2 (CDR2) comprising an amino acid sequence selected from the group consisting of SEQ ID NO: 18, 27, 31, 42, 44, 48, 52, 61, 63, 71, 73, 75, 77, and 79; and a complementarity determining region 3 (CDR3) comprising an amino acid sequence selected from the group consisting of SEQ ID NO: 19, 22, 24, 28, 32, 55, and 57.

In any of the above embodiments, at least one BD comprises an amino acid sequence selected from the group consisting of SEQ ID NO: 100, 104, 108, 112, 114, 116, and 119-124.

In any of the above embodiments, at least one BD comprises an amino acid sequence selected from the group consisting of SEQ ID NO: 119-124.

In any of the above embodiments, at least one BD comprises a CDR1 comprising an amino acid sequence selected from the group consisting of SEQ ID NO: 101, 105, and 109; a CDR2 comprising an amino acid sequence selected from the group consisting of SEQ ID NO: 102, 106, 110, and 117; and a CDR3 comprising an amino acid sequence selected from the group consisting of SEQ ID NO: 103, 107, 111, 113, 115, and 118.

In any of the above embodiments, at least one TBD comprises an amino acid sequence selected from the group consisting of SEQ ID NO: 16, 20, 23, 25, 29, 33, 39, 33-41, 43, 45-47, 49, 51, 53, 54, 56, 58-60, 62, 65, 66, 68, 70, 72, 74, 76, 78, and 80-83, and at least one BD comprises an amino acid sequence selected from the group consisting of SEQ ID NO: 100, 104, 108, 112, 114, 116, and 119-124.

In any of the above embodiments, at least one TBD comprises an amino acid sequence selected from the group consisting of SEQ ID NO: 33, 39, 33-41, 43, 45-47, 49, 51, 53, 54, 56, 58-60, 62, 65, 66, 68, 70, 72, 74, 76, 78, and 80-83, and at least one BD comprises an amino acid sequence selected from the group consisting of SEQ ID NO: 119-124.

In any of the above embodiments, at least one TBD comprises a complementarity determining region 1 (CDR1) comprising an amino acid sequence selected from the group consisting of SEQ ID NO: 17, 21, 26, 30, 50, 65, and 69; a complementarity determining region 2 (CDR2) comprising an amino acid sequence selected from the group consisting of SEQ ID NO: 18, 27, 31, 42, 44, 48, 52, 61, 63, 71, 73, 75, 77, and 79; and a complementarity determining region 3 (CDR3) comprising an amino acid sequence selected from the group consisting of SEQ ID NO: 19, 22, 24, 28, 32, 55, and 57, and at least one BD comprises a CDR1 comprising an amino acid sequence selected from the group consisting of SEQ ID NO: 101, 105, and 109; a CDR2 comprising an amino acid sequence selected from the group consisting of SEQ ID NO: 102, 106, 110, and 117; and a CDR3 comprising an amino acid sequence selected from the group consisting of SEQ ID NO: 103, 107, 111, 113, 115, and 118.

### BRIEF DESCRIPTION OF THE DRAWINGS

FIG. 1 is schematic of exemplary multivalent and multispecific fusion proteins of the present disclosure.

FIGS. 2A and 2B are a pair of graphs demonstrating the ability of 41BB single domain antibodies (sdAbs) to bind recombinant human 41BB (FIG. 2A) or cyno 41BB (FIG. 2B). Binding was assessed by ELISA wherein recombinant 41BB-mFc protein was immobilized on a Medisorp 96 well plate.

FIG. 3 is a graph demonstrating the ability of 41BB single domain antibodies (sdAbs) to bind cell surface 41BB. Binding was assessed by flow cytometry using 41BB expressing CHO cells and data is presented as median fluorescence intensity.

FIG. 4 is a graph demonstrating the ability of 41BB single domain antibodies, RH3 and 4H04 to bind cynomolgus monkey 41BB. Binding was assessed by ELISA wherein recombinant 41BB-mFc protein was immobilized on a Medisorp 96 well plate.

FIG. 5 is a graph demonstrating the capacity of 41BB single domain antibodies (VHHs) to block the interaction between 41BB and 41BBL. All single domain antibodies tested, with the exception of RH3 blocks the interaction between 41BB and 41BBL. Blocking was assessed by flow cytometry using a recombinant 41BB fusion protein and 41BB expressing CHO cells, data is presented as median fluorescence intensity.

FIG. 6 is a graph demonstrating the inability of a conventional bivalent anti-41BB antibody PF-05082566 to induce 41BB signaling unless further clustered with an exogenous crosslinking anti-human IgG antibody. 41BB signaling was monitored using a NF-kB reporter 293 cell line expressing 41BB.

FIGS. 7A and 7B are a pair of graphs demonstrating the capacity of an exemplary PDL1 single domain antibody (28A10) to bind cell surface PDL1 and to block the interaction with PD1. Binding (FIG. 7A) was assessed by flow cytometry on PDL1 expressing CHO cells. Blocking (FIG. 7B) was assessed by flow cytometry using a recombinant PD1 fusion protein and PDL1 expressing CHO cells, data is presented as median fluorescence intensity.

FIGS. 8A, 8B, and 8C are a series of illustrations and a graph depicting PDL1-dependent 41BB agonism mediated by bispecific PDL1-41BB targeting fusion proteins of the present disclosure. FIGS. 8A and 8B are conceptual schematics, wherein the bispecific fusion proteins have minimal 41BB agonistic properties (FIG. 8A) unless bound by a PD-L1 expressing cell (FIG. 8B). FIG. 8C is a graph demonstrating the ability of a PDL1-positive cell (here PDL1 transfected CHO cells) to mediate 41BB signaling and the inability of PDL1-negative cell (here untransfected CHO cells) to mediate 41BB signaling. 41BB signaling was monitored using a NF-kB reporter 293 cell line expressing 41BB.

FIGS. 9A, 9B, 9C, 9D, and 9E are a series of graphs demonstrating the binding to human (FIG. 9A and FIG. 9C) or cynomolgus monkey (FIG. 9B and FIG. 9D) 41BB of humanized RH3 variants. Binding was assessed by flow cytometry on 41BB expressing 293freestyle cells. FIG. 9E is a graph demonstrating that the humanized variants hzRH3v5-1 and hzRH3v9 do not block binding of 41BBL to cell surface 41BB. Herein a recombinant fusion protein 41BBL-mFc, containing a mouse Fc region was used and bound 41BBL was detected using an anti-mouse IgG-Fc specific secondary antibody.

FIG. 10 is a graph demonstrating the specific binding of hzRH3v5-1 (40 nM) to 41BB compared to other TNFRSF members OX40 and GITR. Binding was assessed by flow cytometry using CHO cells expressing the given TNFRSF member.

FIGS. 11A, 11B, 11C, and 11D are a series of graphs demonstrating the binding to human (FIG. 11A and FIG. 11C) or cynomolgus monkey (FIG. 11B) 41BB of humanized 4E01 variants. Binding was assessed by flow cytometry on 41BB expressing 293freestyle cells. FIG. 11D is a graph demonstrating that the humanized variants hz4E01v16, hz4EO1v18, hz4EO1v21, hz4E01v22 and hz4E01v23 block binding of 41BBL to cell surface 41BB. In these studies, a recombinant fusion protein 41BBL-mFc, containing a mouse Fc region was used and bound 41BBL was detected using an anti-mouse IgG-Fc specific secondary antibody.

FIG. 12 is a graph demonstrating binding of humanized single domain antibodies targeting PDL1. Binding was assessed by flow cytometry on PDL1-expressing CHO cells.

FIG. 13 is a schematic of two exemplary formats of a PDL1×41BB bispecific, INBRX-105-1. INBRX-105-1-A (left) has the PDL1 and 41BB binding domains, located at opposing terminal positions with a central Fc region, whereas INBRX-105-1-B (right) has the PDL1 and 41BB binding domains positioned in tandem, N-terminal to an Fc region.

FIGS. 14A, 14B, and 14C are a series of graphs demonstrating the equivalent binding to human (FIG. 14A) or cynomolgus monkey (FIG. 14B) 41BB by the two distinct formats of a bispecific fusion protein targeting PDL1 and 41BB referred to herein as INBRX-105-1-A and INBRX-105-1-B. Binding was assessed by flow cytometry on 41BB expressing 293freestyle cells. FIG. 14C is a graph that demonstrates that the bispecific fusion protein containing hzRh3v5-1 does not block 41BBL binding to cell surface 41BB. Herein a recombinant fusion protein of 41BBL and mouse Fc region was used and bound 41BBL was detected using an anti-mouse IgG-Fc specific secondary antibody.

FIGS. 15A, 15B, 15C, and 15D are a series of graphs demonstrating the equivalent binding (FIG. 15A and FIG. 15C). and PD1 blocking (FIG. 15B and FIG. 15D) by the two distinct formats of a bispecific fusion protein targeting PDL1 and 41BB referred to herein as INBRX-105-1-A and INBRX-105-1-B. Binding was assessed by flow cytometry on human (FIG. 15A) or cynomolgus monkey (FIG. 15C) PDL1 expressing 293freestyle cells. Blocking was assessed by flow cytometry using on human (FIG. 15B) or cynomolgus monkey (FIG. 15D) PDL1 expressing 293freestyle cells with either recombinant human (FIG. 15B) or cynomolgus monkey (FIG. 15D) PD1-mFc fusion protein. Bound PD1 was detected using an anti-mouse IgG-Fc specific secondary antibody.

FIG. 16 is a graph demonstrating the ability of humanized versions of a PDL1×41BB bispecific fusion protein (INBRX-105-1) to induce PDL1-dependent 41BB agonism. A 41BB-expressing HEK293 NF-kB reporter cell line was used to assess 41BB signaling and a PDL1-expressing CHO cell line was used as the source of PDL1.

FIGS. 17A and 17B are a pair of graphs demonstrating the 41BB-specific binding by the 41BB-binding portion of a PDL1×41BB bispecific fusion protein (INBRX-105-1) of the present disclosure. Binding was assessed on 41BB (FIG. 17A) or the closest homolog, TNFRSF21/DR6 (FIG. 17B), expressing 293freestyle cells by flow cytometry. An anti-DR6 antibody (Invitrogen) was used to as positive control for DR6 expression.

FIGS. 18A, 18B, and 18C are a series of graphs demonstrating the PDL1-specific binding by the PDL1-binding portion of a PDL1×41BB bispecific fusion protein (INBRX-105-1) of the present disclosure. Binding was assessed on PDL1 (FIG. 18A), and its closest homologs PDL2 (FIG. 18B) or VISTA/PDL3 (FIG. 18C), expressing 293freestyle cells by flow cytometry. Anti-PDL2 and anti-VISTA antibodies were used to as positive controls for PDL2 and PDL3 expression respectively.

FIGS. 19A and 19B are a pair of graphs demonstrating the ability of a PDL1×41BB bispecific fusion protein to simultaneously bind PDL1 and 41BB. Bound 41BB was detected using an anti-mouse IgG-Fc specific secondary antibody. FIG. 19A. is a graph showing the binding of INBRX-105-1 to the PDL1 expressing K562 cells. FIG. 19B is a graph showing the binding of recombinant 41BB to INBRX-105-1 on the PDL1 expressing cells.

FIG. 20 is a graph demonstrating the ability of a PDL1×41BB bispecific fusion protein to simultaneously bind recombinant PDL1 and recombinant 41BB in an ELISA. Bound recombinant 41BB was detected via streptavidin-HRP.

FIGS. 21A, 21B, and 21C are a series of graphs demonstrating the effect of a PDL1×41BB bispecific fusion protein (INBRX-105-1) of the present disclosure on T-cell activation and proliferation. INFγ production in the cell supernatant was monitored using an ELISA and normalized to the standard curve. T-cell proliferation was monitored by flow cytometry using CTV labeling of T-cells. T-cell activation was assessed by the presence of the activation marker CD25 monitored by flow cytometry. Antibodies were used at 10 nM.

FIGS. 22A and 22B are a pair of graphs demonstrating PDL1-dependent 41BB agonism mediated by a PDL1×41BB bispecific fusion protein (INBRX-105-1) of the present disclosure. CD8⁺ T-cell proliferation (FIG. 22A) was monitored using CTV labeling and INFγ production (FIG. 22B) in the cell supernatant was monitored using an ELISA and normalized to the standard curve.

FIG. 23 is a graph demonstrating the capacity of a PDL1×41BB bispecific fusion protein (INBRX-105-1) of the present disclosure to enhance the Th1 lineage defining transcription factor, T-bet, expression in T-cell populations. T-bet expression was assessed on CD4+ and CD8⁺ T-cell population by flow cytometry via intracellular staining following fixation and permeabilization.

FIGS. 24A and 24B are a pair graphs contrasting the capacity of a PDL1×41BB bispecific fusion protein (INBRX-105-1) of the present disclosure and the combination of monospecific antibodies Atezolizumab (anti-PDL1) and Utomilumab (anti-41BB) to induce INFγ (FIG. 24A) or TNFα (FIG. 24B) production from CD4+ or CD8⁺ T-cells. Cytokine expression was assessed on CD4+ and CD8⁺ T-cell population by flow cytometry via intracellular staining following fixation and permeabilization.

FIGS. 25A and 25B are a pair of graphs demonstrating the agonistic capacity of a tetravalent 41BB-binding fusion protein and PDL1×41BB bispecific fusion proteins of the present disclosure in the presence of an additional PDL1 positive (FIG. 25A) or negative (FIG. 25B) cell line. Herein a 41BB-expressing HEK293 NF-kB reporter cell was used and co-incubated with either the PDL1-negative K562 cell line (FIG. 25B) or a stably transfected, PDL1-expressing K562 cell line (FIG. 25A).

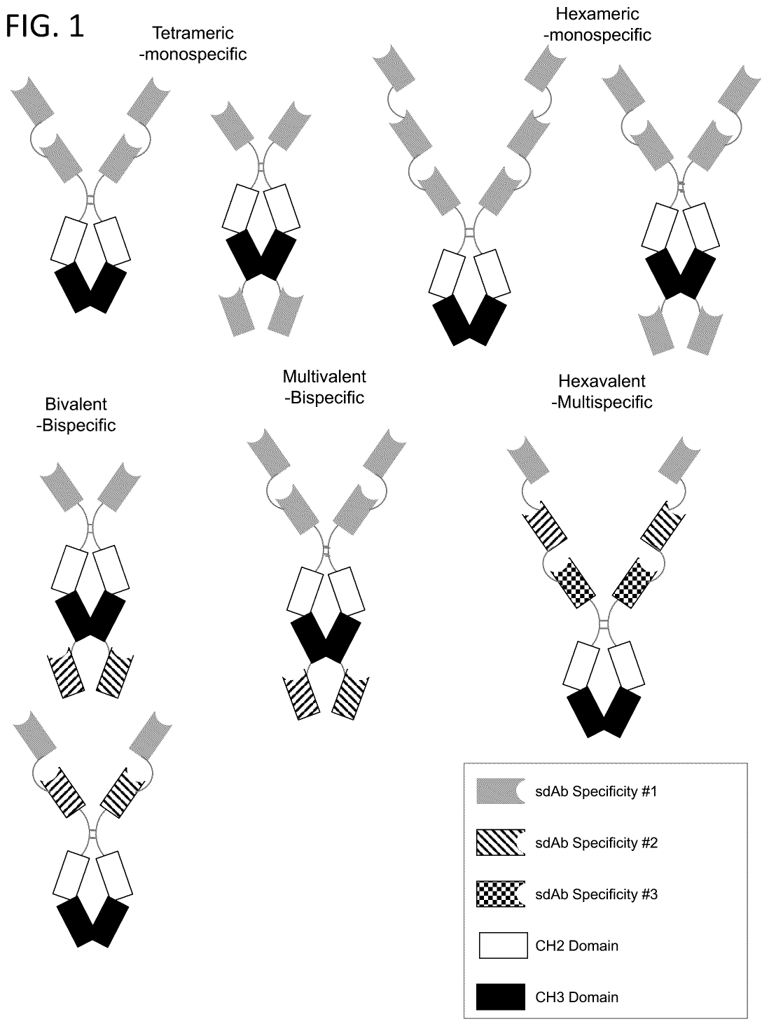

### DETAILED DESCRIPTION OF THE INVENTION

All patents and publications mentioned in this specification are herein incorporated by reference to the same extent as if each independent patent and publication was specifically and individually indicated to be incorporated by reference.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the terms “dual-targeting fusion protein” and “antibody” can be synonyms. As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, Fab, Fab′ and F(ab′)₂ fragments, Fv, scFvs, a Fab expression library, and single domain antibody (sdAb) fragments, for example V_{H}H, V_{NAR}, engineered V_{H} or V_{K}.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses (also known as isotypes) as well, such as IgG₁, IgG₂, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The single domain antibody (sdAb) fragments portions of the fusion proteins of the present disclosure are referred to interchangeably herein as targeting polypeptides herein.

As used herein, the term “epitope” includes any protein determinant capable of specific binding to/by an immunoglobulin or fragment thereof, or a T-cell receptor. The term “epitope” includes any protein determinant capable of specific binding to/by an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 mM, for example, ≤1 μM; e.g., ≤100 nM, for example, ≤10 nM and for example, ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kₒₙ) and the “off rate constant” (k_{off}) can be determined by calculation of the concentrations and the actual rates of association and dissociation. (See Nature 361:186-87 (1993)). The ratio of k_{off}/kₒ enables the cancellation of all parameters not related to affinity, and is equal to the dissociation constant K_{d}. (See, generally, Davies et al. (1990) Annual Rev Biochem 59:439-473). An antibody of the present disclosure is said to specifically bind to an antigen, when the equilibrium binding constant (K_{d}) is 1 M, for example, ≤100 nM, for example, 10 nM, and for example, 100 μM to about 1 μM, as measured by assays such as radioligand binding assays, surface plasmon resonance (SPR), flow cytometry binding assay, or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide,” as referred to herein, refers to a polymeric boron of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In some embodiments, oligonucleotides are 10 to 60 bases in length and for example, 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the disclosure are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

The term “selectively hybridize” referred to herein means to detectably and specifically bind. Polynucleotides, oligonucleotides and fragments thereof in accordance with the disclosure selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the disclosure and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, σ-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, for example, at least 90 percent sequence identity, for example, at least 95 percent sequence identity, and for example, at least 99 percent sequence identity.

In some embodiments, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Suitable conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, for example, at least 80%, 90%, 95%, and for example, 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Suitable amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (for example, conservative amino acid substitutions) may be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, for example, at least 14 amino acids long, for example, at least 20 amino acids long, usually at least 50 amino acids long, and for example, at least 70 amino acids long. The term “analog” as used herein refers to polypeptides which are comprised of a segment of at least 25 amino acids that has substantial identity to a portion of a deduced amino acid sequence and which has specific binding to CD47, under suitable binding conditions. Typically, polypeptide analogs comprise a conservative amino acid substitution (or addition or deletion) with respect to the naturally-occurring sequence. Analogs typically are at least 20 amino acids long, for example, at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p.392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, and/or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, 0-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, the terms “treat,” treating,” “treatment,” and the like refer to reducing and/or ameliorating a disorder and/or symptoms associated therewith. By “alleviate” and/or “alleviating” is meant decrease, suppress, attenuate, diminish, arrest, and/or stabilize the development or progression of a disease such as, for example, a cancer. It will be appreciated that, although not precluded, treating a disorder or condition does not require that the disorder, condition or symptoms associated therewith be completely eliminated.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present.

Generally, a substantially pure composition will comprise more than about 80 percent of all macromolecular species present in the composition, for example, more than about 85%, 90%, 95%, and 99%. In some embodiments, the object species is purified to essential homogeneity (contaminant species cannot be detected in the composition by conventional detection methods) wherein the composition consists essentially of a single macromolecular species.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

By “effective amount” is meant the amount required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “subject” is meant a mammal, including, but not limited to, a human or non-human mammal, such as a bovine, equine, canine, rodent, ovine, primate, camelid, or feline.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration.

By “fragment” is meant a portion of a polypeptide or nucleic acid molecule. This portion contains, for example, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” 41BB (CD137, TNFRSF9) Targeting

41BB is a member of the TNF receptor superfamily that is predominately expressed on activated T-cells and NK cells and serves as a co-stimulatory molecule. Agonizing 41BB enhances T cell proliferation and survival, cytolytic activity and cytokine secretion (e.g., IL-2, TNFα and INFγ). In mice, 41BB engagement has been shown to enhance anti-tumor immunity. (Croft, 2009, Nat Rev Immunol 9:271-285; Lynch, 2008, Immunol Rev. 22: 277-286). Importantly, tumor infiltrating cytotoxic T-cells (CTLs), have been shown to be express 41BB and it is these 41BB positive CTLs that have the highest anti-tumor cytotoxic activity (Ye et al Clin Cancer Res; 20(1): 44-55). The ligand for 41BB, 41BBL, naturally forms a homotrimer any thereby suggests that signaling is mediated by higher order clustering of 41BB. This is activation mechanism is shared with many members of the TNFRSF. Interest in exploiting 41BB signaling for anti-tumor immunotherapy has prompted the development of therapeutic 41BB antibodies. However, the capacity of bivalent 41BB antibodies to induce signaling is weak in absence of an exogenous clustering event. This can be achieved to some degree through the interaction with Fcγ-receptors (FcγRs), yet this can also lead to depletion of the 41BB-expressing cell through effector mechanisms (e.g. ADCC and ADCP). Furthermore, competition with the high concentration of IgG in serum attenuates efficient FcγR interactions. Therefore, current bivalent antibodies targeting 41BB are either ineffective agonists or have the liability of depleting the vary cells wherein 41BB signaling is desired. It has previously been shown that the therapeutic 41BB antibody, PF-05082566 is only capable of mediated 41BB signaling with cross-linked with anti-human secondary antibody (Fisher et al Cancer Immunol Immunother (2012) 61:1721-1733). Therefore, there exists a need for optimized 41BB agonist capable of mediating signaling in the absence of an exogenous crosslinking agent or FcγR interaction. The fusion proteins of the present disclosure are capable of mediating potent 41BB signaling 1) without any additional interactions when formatted as a multivalent fusion protein or 2) conditionally when engaged with at least a second antigen interaction when formatted as a multispecific fusion protein. The fusion proteins of the present disclosure are capable of standalone (multivalent) or conditional (multispecific) co-stimulatory activity on T-cell and NK cells.

Exemplary amino acid sequences of 41BB binding single domain antibodies are shown below:

4H04:
(SEQ ID NO: 16)

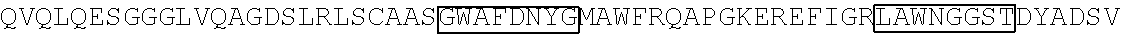

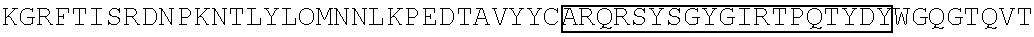

(SEQ ID NO: 17)
CDR1: GWAFDNYG
(SEQ ID NO: 18)
CDR2: LAWNGGST
(SEQ ID NO: 19)
CDR3: ARQRSYSGYGIRTPQTYDY
4E1:
(SEQ ID NO: 20)

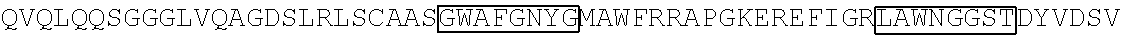

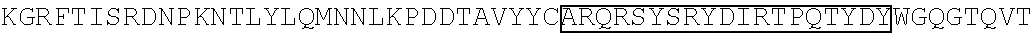

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
4F5:
(SEQ ID NO: 23)

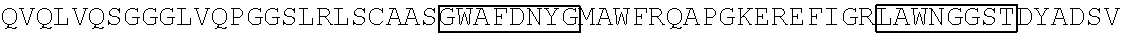

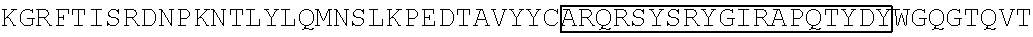

(SEQ ID NO: 17)
CDR1: GWAFDNYG
(SEQ ID NO: 18)
CDR2: LAWNGGST
(SEQ ID NO: 24)
CDR3: ARQRSYSRYGIRAPQTYDY
RH3:
(SEQ ID NO: 25)

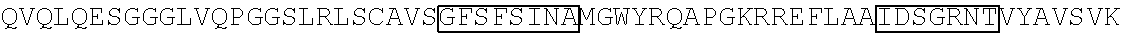

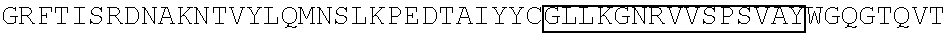

(SEQ ID NO: 26)
CDR1: GFSFSINA
(SEQ ID NO: 27)
CDR2: IDSGRNT
(SEQ ID NO: 28)
CDR3: GLLKGNRVVSPSVAY
D1:
(SEQ ID NO: 29)

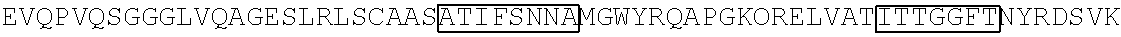

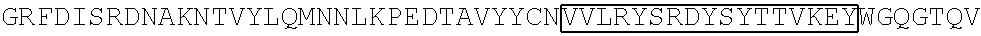

(SEQ ID NO: 30)
CDR1: ATIFSNNA
(SEQ ID NO: 31)
CDR2: ITTGGFT
(SEQ ID NO: 32)
CDR3: NVVLRYSRDYSYTTVKEY
1G3:
(SEQ ID NO: 432)

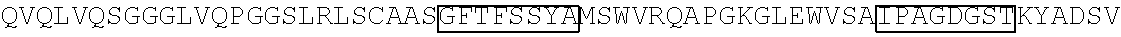

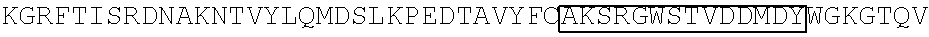

(SEQ ID NO: 433)
CDR1: GFTFSSYA
(SEQ ID NO: 434)
CDR2: IPAGDGST
(SEQ ID NO: 435)
CDR3: AKSRGWSTVDDMDY
1H4:
(SEQ ID NO: 436)

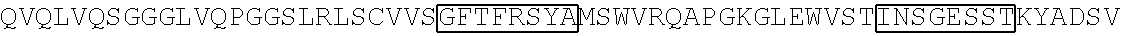

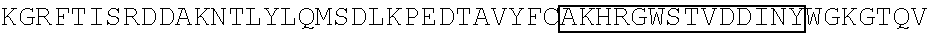

(SEQ ID NO: 437)
CDR1: GFTERSYA
(SEQ ID NO: 438)
CDR2: INSGESST
(SEQ ID NO: 439)
CDR3: AKHRGWSTVDDINY
1H1:
(SEQ ID NO: 440)

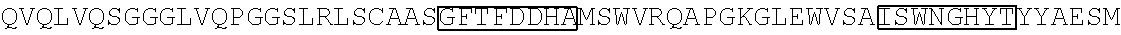

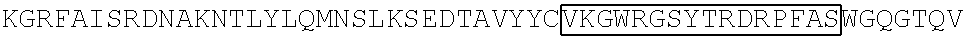

(SEQ ID NO: 441)
CDR1: GFTFDDHA
(SEQ ID NO: 442)
CDR2: ISWNGHYT
(SEQ ID NO: 443)
CDR3: VKGWRGSYTRDRPFAS
1H8:
(SEQ ID NO: 444)

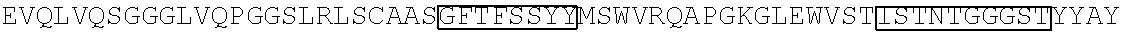

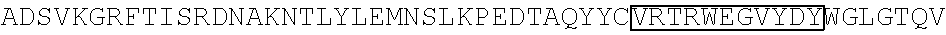

(SEQ ID NO: 445)
CDR1: GFTFSSYY
(SEQ ID NO: 446)
CDR2: ISTNTGGGST
(SEQ ID NO: 447)
CDR3: VRTRWEGVYDY
Hz4E1-v1:
(SEQ ID NO: 33)

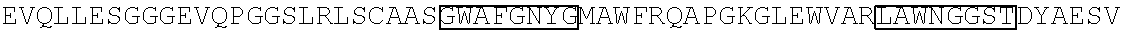

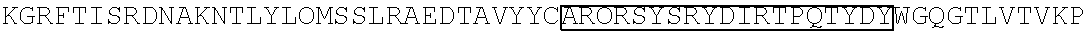

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
Hz4E1-v3:
(SEQ ID NO: 34)

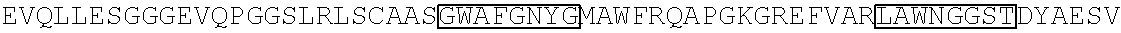

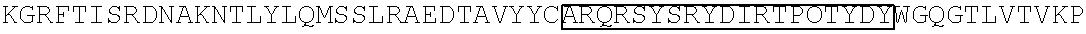

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v7-1:
(SEQ ID NO: 35)

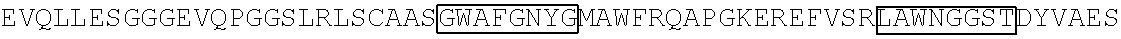

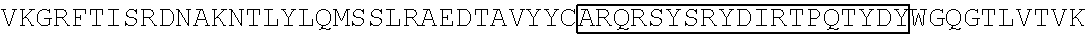

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v8:
(SEQ ID NO: 36)

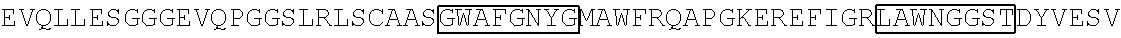

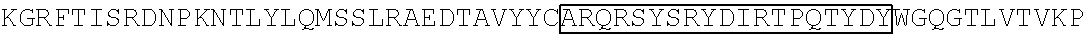

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v9:
(SEQ ID NO: 37)

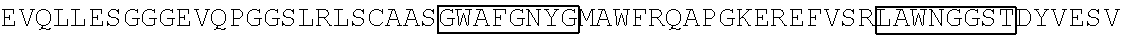

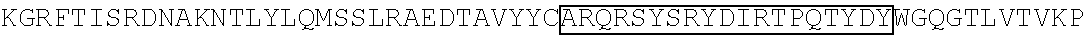

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v10:
(SEQ ID NO: 38)

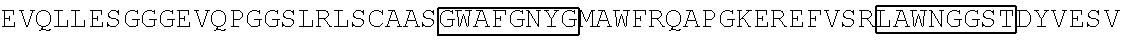

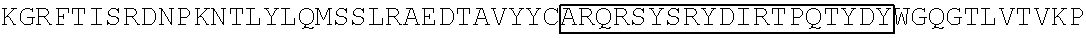

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v11:
(SEQ ID NO: 39)

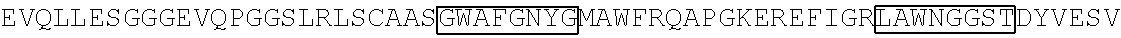

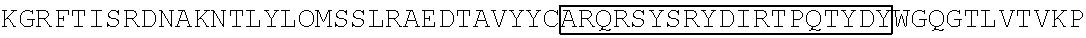

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v12:
(SEQ ID NO: 40)

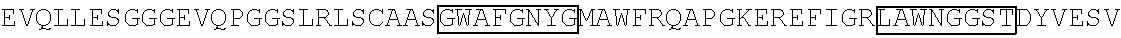

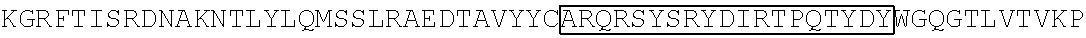

(SEQ ID NO: 21)
CDR1: GWAFGNYG
(SEQ ID NO: 18)
CDR2: LAWNGGST
(SEQ ID NO: 22)
CDR3: ARQRSYSRYDIRTPQTYDY
hz4E01v13:
(SEQ ID NO: 41)

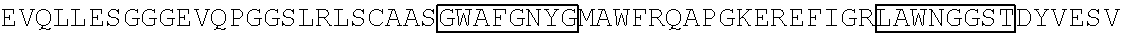

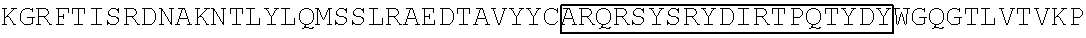

(SEQ ID NO: 21)
CDR1: GWAFGNYG
(SEQ ID NO: 42)
CDR2: LAWQGGST
(SEQ ID NO: 22)
CDR3: ARQRSYSRYDIRTPQTYDY
hz4E01v14:
(SEQ ID NO: 43)

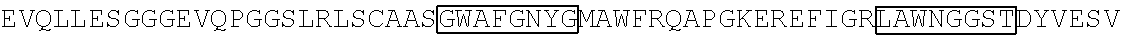

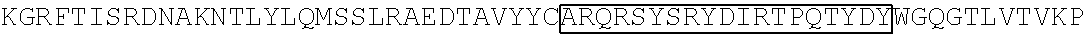

(SEQ ID NO: 21)
CDR1: GWAFGNYG
(SEQ ID NO: 44)
CDR2: LAWNAGST
(SEQ ID NO: 22)
CDR3: ARQRSYSRYDIRTPQTYDY
hz4E01v16:
(SEQ ID NO: 43)

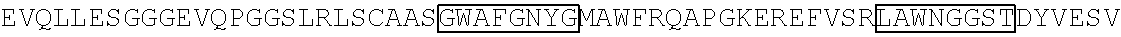

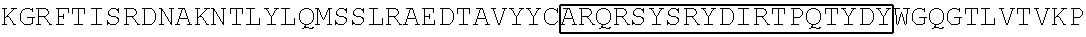

(SEQ ID NO: 21)
CDR1: GWAFGNYG
(SEQ ID NO: 42)
CDR2: LAQGGST
(SEQ ID NO: 22)
CDR3: ARQRSYSRYDIRTPQTYDY
hz4E01v17:
(SEQ ID NO: 46)

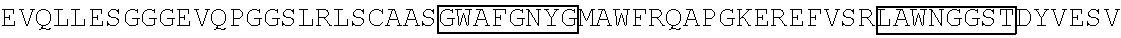

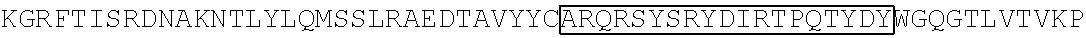

(SEQ ID NO: 21)
CDR1: GWAFGNYG
(SEQ ID NO: 44)
CDR2: LAWNAGST
(SEQ ID NO: 22)
CDR3: ARQRSYSRYDIRTPQTYDY
hz4E01v18:
(SEQ ID NO: 47)

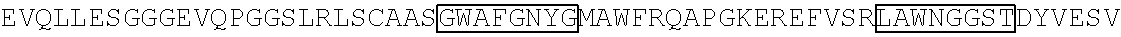

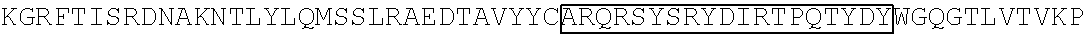

(SEQ ID NO: 21)
CDR1: GWAFGNYG
(SEQ ID NO: 48)
CDR2: LAWGGGST
(SEQ ID NO: 22)
CDR3: ARQRSYSRYDIRTPQTYDY
hz4E01v21:
(SEQ ID NO: 49)

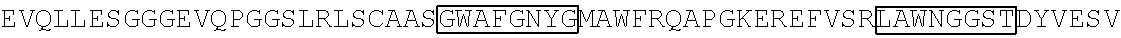

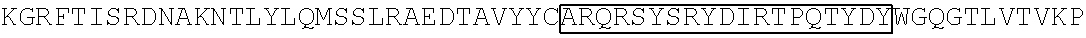

(SEQ ID NO: 50)
CDR1: GWAFSNYG
(SEQ ID NO: 48)
CDR2: LAWGGGST
(SEQ ID NO: 22)
CDR3: ARQRSYSRYDIRTPQTYDY
hz4E01v22:
(SEQ ID NO: 47)

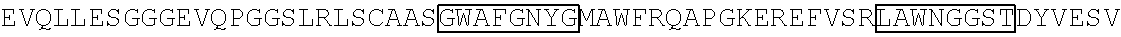

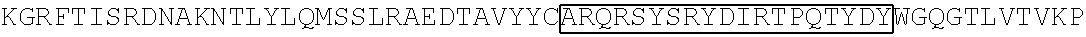

(SEQ ID NO: 21)
CDR1: GWAFGNYG
(SEQ ID NO: 52)
CDR2: LAWSGGST
(SEQ ID NO: 22)
CDR3: ARQRSYSRYDIRTPQTYDY
hz4E01v23:
(SEQ ID NO: 53)

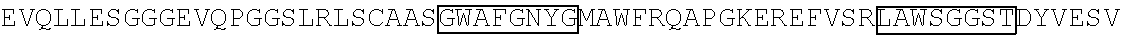

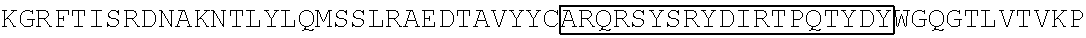

(SEQ ID NO: 50)
CDR1: GWAFSNYG
(SEQ ID NO: 52)
CDR2: LAWSGGST
(SEQ ID NO: 22)
CDR3: ARQRSYSRYDIRTPQTYDY
hz4E01v24:
(SEQ ID NO: 47)

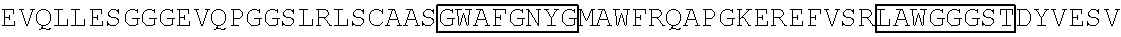

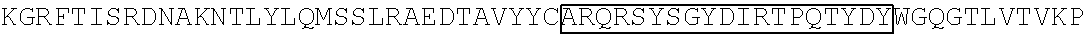

(SEQ ID NO: 21)
CDR1: GWAFGNYG
(SEQ ID NO: 48)
CDR2: LAWGGGST
(SEQ ID NO: 55)
CDR3: ARQRSYSGYDIRTPQTYDY
hz4E01v25:
(SEQ ID NO: 56)

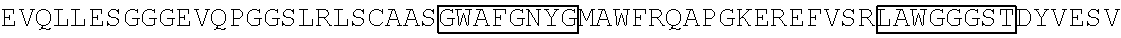

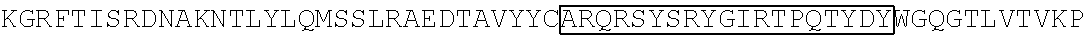

(SEQ ID NO: 21)
CDR1: GWAFGNYG
(SEQ ID NO: 48)
CDR2: LAWGGGST
(SEQ ID NO: 57)
CDR3: ARQRSYSRYGIRTPQTYDY
hz4E01v26:
(SEQ ID NO: 56)

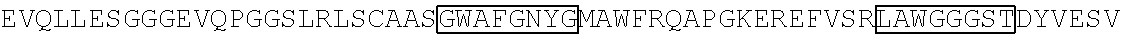

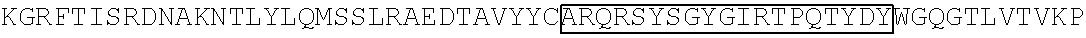

(SEQ ID NO: 21)
CDR1: GWAFGNYG
(SEQ ID NO: 48)
CDR2: LAWGGGST
(SEQ ID NO: 19)
CDR3: ARQRSYSGYGIRTPQTYDY
hzRH3-v1:
(SEQ ID NO: 59)

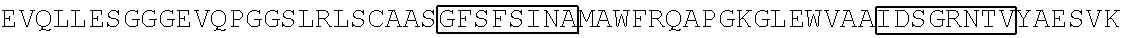

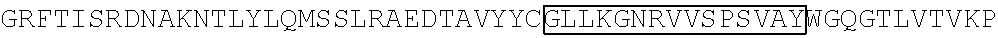

(SEQ ID NO: 26)
CDR1: GFSFSINA
(SEQ ID NO: 27)
CDR2: IDSGRNT
(SEQ ID NO: 28)
CDR3: GLLKGNRVVSPSVAY
hzRH3v5-1:
(SEQ ID NO: 60)

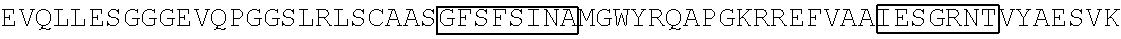

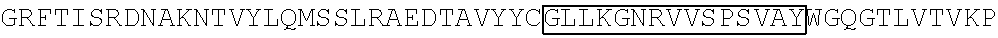

(SEQ ID NO: 26)
CDR1: GFSFSINA
(SEQ ID NO: 61)
CDR2: IESGRNT
(SEQ ID NO: 28)
CDR3: GLLKGNRVVSPSVAY
hzRH3v5-2:
(SEQ ID NO: 62)

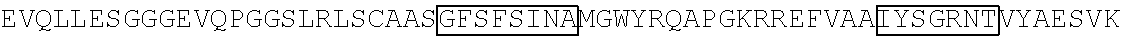

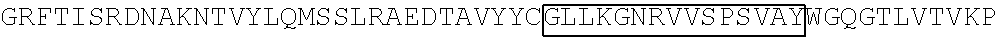

(SEQ ID NO: 26)
CDR1: GFSFSINA
(SEQ ID NO: 63)
CDR2: IYSGRNT
(SEQ ID NO: 28)
CDR3: GLLKGNRVVSPSVAY
hzRH3v5-3
(SEQ ID NO: 64)

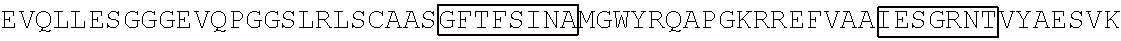

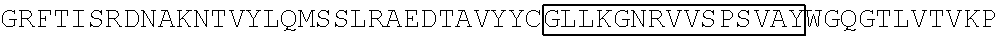

(SEQ ID NO: 65)
CDR1: GFTFSINA
(SEQ ID NO: 61)
CDR2: IESGRNT
(SEQ ID NO: 28)
CDR3: GLLKGNRVVSPSVAY
hzRH3v5-6
(SEQ ID NO: 66)

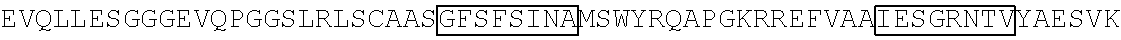

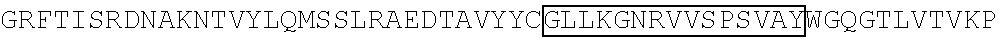

(SEQ ID NO: 67)
CDR1: GFSFSINA
(SEQ ID NO: 61)
CDR2: IESGRNT
(SEQ ID NO: 28)
CDR3: GLLKGNRVVSPSVAY
hzRH3v5-8
(SEQ ID NO: 68)

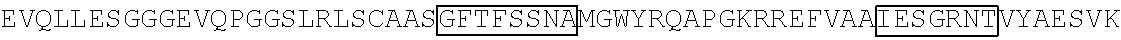

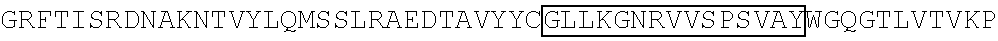

(SEQ ID NO: 69)
CDR1: GFTFSINA
(SEQ ID NO: 61)
CDR2: IESGRNT
(SEQ ID NO: 28)
CDR3: GLLKGNRVVSPSVAY
hzRH3v5-10
(SEQ ID NO: 70)

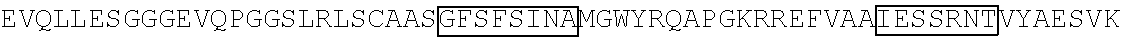

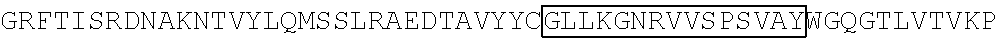

(SEQ ID NO: 26)
CDR1: GFSFSINA
(SEQ ID NO: 71)
CDR2: IESSRNT
(SEQ ID NO: 28)
CDR3: GLLKGNRVVSPSVAY
hzRH3v5-12
(SEQ ID NO: 72)

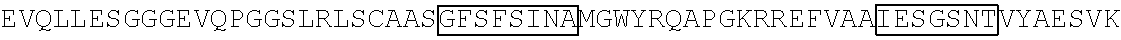

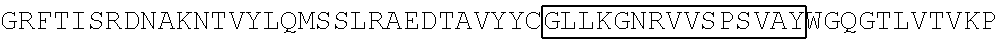

(SEQ ID NO: 26)
CDR1: GFSFSINA
(SEQ ID NO: 73)
CDR2: IESGSNT
(SEQ ID NO: 28)
CDR3: GLLKGNRVVSPSVAY
hzRH3v5-14
(SEQ ID NO: 74)

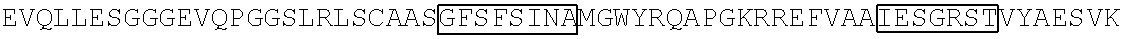

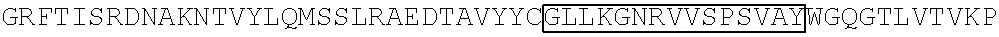

(SEQ ID NO: 26)
CDR1: GFSFSINA
(SEQ ID NO: 75)
CDR2: IESGRNT
(SEQ ID NO: 28)
CDR3: GLLKGNRVVSPSVAY
hzRH3v5-15
(SEQ ID NO: 74)

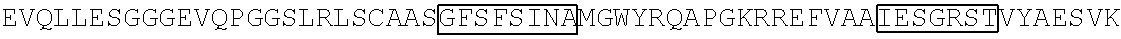

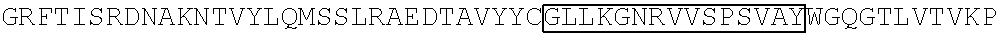

(SEQ ID NO: 26)
CDR1: GFSFSINA
(SEQ ID NO: 75)
CDR2: IESGRNT
(SEQ ID NO: 28)
CDR3: GLLKGNRVVSPSVAY
hzRH3v5-16
(SEQ ID NO: 78)

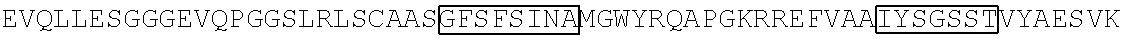

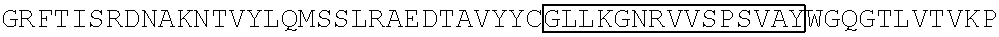

(SEQ ID NO: 26)
CDR1: GFSFSINA
(SEQ ID NO: 79)
CDR2: IYSGRNT
(SEQ ID NO: 28)
CDR3: GLLKGNRVVSPSVAY
hzRH3v7
(SEQ ID NO: 80)

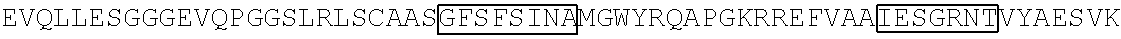

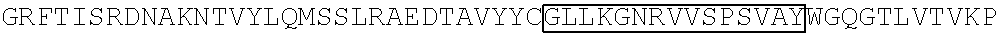

(SEQ ID NO: 26)
CDR1: GFSFSINA
(SEQ ID NO: 61)
CDR2: IESGRNT
(SEQ ID NO: 28)
CDR3: GLLKGNRVVSPSVAY
hzRH3v8
(SEQ ID NO: 81)

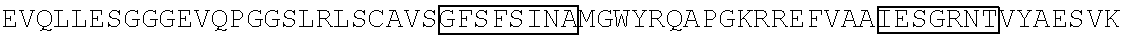

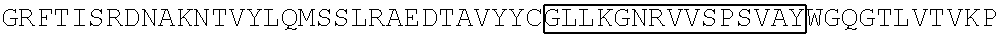

(SEQ ID NO: 26)
CDR1: GFSFSINA
(SEQ ID NO: 61)
CDR2: IESGRNT
(SEQ ID NO: 28)
CDR3: GLLKGNRVVSPSVAY
hzRH3v9
(SEQ ID NO: 82)

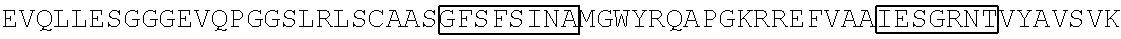

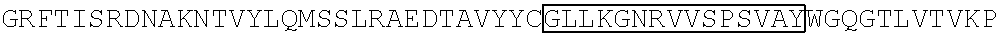

(SEQ ID NO: 26)
CDR1: GFSFSINA
(SEQ ID NO: 61)
CDR2: IESGRNT
(SEQ ID NO: 28)
CDR3: GLLKGNRVVSPSVAY
hzRH3v13
(SEQ ID NO: 83)

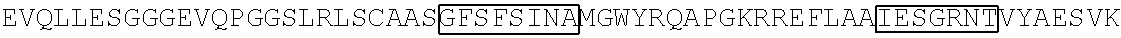

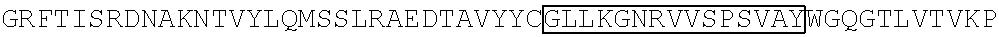

(SEQ ID NO: 26)
CDR1: GFSFSINA
(SEQ ID NO: 61)
CDR2: IESGRNT
(SEQ ID NO: 28)
CDR3: GLLKGNRVVSPSVAY

In some embodiments, the 41BB binding domain comprises or is derived from an antibody sequence or antigen-binding fragment thereof that includes a combination of a variable heavy chain (VH) sequence and a variable light chain (VL) sequence selected from the group consisting of:

VH Sequences:
(SEQ ID NO: 84)
QVQLVQSGAEVKKPGSSVKVSCKASGGTFNSYAISWVRQAPGQGLEWMGG
IIPGFGTANYAQKFQGRVTITADESTSTAYMELSSLRSEDTAVYYCARKN
EEDGGFDHWGQGTLVTVSS
(SEQ ID NO: 85)
QVQLVESGGGLVQPGGSLRLSCAASGFTFSDYYMHWVRQAPGKGLEWVSV
ISGSGSNTYYADSVKGRFTISRDNSKNTLYLQMNSLRAEDTAVYYCARLY
AQFEGDFWGQGTLVTVSS
(SEQ ID NO: 86)
QVQLVQSGAEVKKPGESLKISCKGSGYSFSTYWISWVRQMPGKGLEWMGK
IYPGDSYTNYSPSFQGQVTISADKSISTAYLQWSSLKASDTAMYYCARGY
GIFDYWGQGTLVTVSS
(SEQ ID NO: 87)
EVQLVQSGAEVKKPGESLRISCKGSGYSFSTYWISWVRQMPGKGLEWMGK
IYPGDSYTNYSPSFQGQVTISADKSISTAYLQWSSLKASDTAMYYCARGY
GIFDYWGQGTLVTVSS
VL Sequences:
(SEQ ID NO: 88)
DIELTQPPSVSVAPGQTARISCSGDNLGDYYASWYQQKPGQAPVLVIYDD
SNRPSGIPERFSGSNSGNTATLTISGTQAEDEADYYCQTWDGTLHFVFGG
GTKLTVL
(SEQ ID NO: 89)
DIELTQPPSVSVAPGQTARISCSGDNIGSKYVSWYQQKPGQAPVLVIYSD
SERPSGIPERFSGSNSGNTATLTISGTQAEDEADYYCQSWDGSISRVFGG
GTKLTVL
(SEQ ID NO: 90)
DIELTQPPSVSVAPGQTARISCSGDNIGDQYAHWYQQKPGQAPVVVIYQD
KNRPSGIPERFSGSNSGNTATLTISGTQAEDEADYYCATYTGFGSLAVFG
GGTKLTVL
(SEQ ID NO: 91)
SYELTQPPSVSVSPGQTASITCSGDNIGDQYAHWYQQKPGQSPVLVIYQD
KNRPSGIPERFSGSNSGNTATLTISGTQAMDEADYYCATYTGFGSLAVFG
GGTKLTVL
(SEQ ID NO: 92)
SYELTQPPSVSVSPGQTASITCSGDNIGDQYAHWYQQKPGQSPVVVIYQD
KNRPSGIPERFSGSNSGNTATLTISGTQAMDEADYYCATYTGFGSLAVFG
GGTKLTVL
(SEQ ID NO: 93)
DIELTQPPSVSVAPGQTARISCSGDNIGDQYAHWYQQKPGQAPVVVIYQD
KNRPSGIPERFSGSNSGNTATLTISGTQAEDEADYYCSTYTFVGFTTVFG
GGTKLTVL
(SEQ ID NO: 94)
SYELTQPPSVSVSPGQTASITCSGDNIGDQYAHWYQQKPGQSPVLVIYQD
KNRPSGIPERFSGSNSGNTATLTISGTQAMDEADYYCSTYTFVGFTTVFG
GGTKLTVL
(SEQ ID NO: 95)
SYELTQPPSVSVSPGQTASITCSGDNIGDQYAHWYQQKPGQSPVVVIYQD
KNRPSGIPERFSGSNSGNTATLTISGTQAMDEADYYCSTYTFVGFTTVFG
GGTKLTVL

In some embodiments, the 41BB binding domain comprises or is derived from an antibody sequence or antigen-binding fragment thereof that includes a combination of a heavy chain (HC) sequence and a light chain (LC) sequence selected from the group consisting of.

HC Sequences:
(SEQ ID NO: 96)
QVQLQQWGAGLLKPSETLSLTCAVYGGSFSGYYWSWIRQSPEKGLEWIGE
INHGGYVTYNPSLESRVTISVDTSKNQFSLKLSSVTAADTAVYYCARDYG
PGNYDWYFDLWGRGTLVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
(SEQ ID NO: 97)
QVQLQQWGAGLLKPSETLSLTCAVYGGSFSGYYWSWIRQSPEKGLEWIGE
INHGGYVTYNPSLESRVTISVDTSKNQFSLKLSSVTAADTAVYYCARDYG
PGNYDWYFDLWGRGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKENWYVDGVEVHNAKTKPREEQY
NSTYRVVSVLTVLHQDWINGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
LC Sequences:
(SEQ ID NO: 98)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPALTF
GGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSENRGEC

In some embodiments, the 41BB binding domain comprises or is derived from an antibody sequence or antigen-binding fragment thereof selected from the antibody sequences described in US Patent Application Publication No. 20160244528, the contents of which are hereby incorporated by reference in their entirety.

In some embodiments, the 41BB binding domain comprises or is derived from an antibody sequence or antigen-binding fragment thereof selected from the antibody sequences described in U.S. Pat. No. 8,337,850, the contents of which are hereby incorporated by reference in their entirety.

In some embodiments, the 41BB binding domain comprises or is derived from an antibody sequence or antigen-binding fragment thereof selected from the antibody sequences described in PCT Publication No. WO 2005/035584, the contents of which are hereby incorporated by reference in their entirety.

In some embodiments, the 41BB binding domain comprises or is derived from an antibody sequence or antigen-binding fragment thereof selected from the antibody sequences described in EP Patent No. EP 1670828 B1, the contents of which are hereby incorporated by reference in their entirety.

In some embodiments, the 41BB binding domain comprises or is derived from an antibody sequence or antigen-binding fragment thereof selected from the antibody sequences described in PCT Publication No. WO 2006/088447, the contents of which are hereby incorporated by reference in their entirety.

In some embodiments, the 41BB binding domain comprises or is derived from an antibody sequence or antigen-binding fragment thereof selected from the antibody sequences described in US Patent Application Publication No. 20080166336, the contents of which are hereby incorporated by reference in their entirety.

In some embodiments, the 41BB binding domain comprises or is derived from an anti-cancer fusion protein sequence or antigen-binding fragment thereof selected from the sequences described in PCT Publication No. WO 2016/177802, the contents of which are hereby incorporated by reference in their entirety. In some embodiments, the 41BB binding domain comprises or is derived from an amino acid sequence comprising:

(SEQ ID NO: 99)
QDSTSDLIPAPPLSKVPLQQNFQDNQFHGKWYVVGQAGNIRLREDKDPIK
MMATIYELKEDKSYDVTMVKFDDKKCMYDIWTFVPGSQPGEFTLGKIKSF
PGHTSSLVRVVSTNYNQHAMVFFKFVFQNREEFYITLYGRTKELTSELKE
NFIRFSKSLGLPENHIVFPVPIDQCIDG

In some embodiments, the 41BB binding domain comprises or is derived from an 41BB-targeting polypeptide sequence or antigen-binding fragment thereof selected from the sequences described in PCT Publication No. WO 2016/177762, the contents of which are hereby incorporated by reference in their entirety. In some embodiments, the 41BB binding domain comprises or is derived from an amino acid sequence comprising: PDL1 Targeting

In some embodiments, the fusion proteins are multispecific containing at least a first binding domain, e.g., a TBD, and a second binding domain directed toward Program Death Ligand 1 (PD-L1). In these, embodiments, the binding to PD-L1 is capable of providing the additional crosslinking function and TNFRSF activation is achieved with only one or two TBDs. In these embodiments, the TNFRSF signaling is enhanced and focused by the presence of a PD-L1 expressing cell.

PDL1 is a 40 kDa type I transmembrane protein that forms a complex with its receptor programmed cell death protein 1 (PD1), also known as CD279. Engagement of PDL1 with its receptor PD1 on T cells delivers a signal that inhibits TCR-mediated activation of IL-2 production and T cell proliferation. Aberrant expression and/or activity of PDL1 and PDL1-related signaling has been implicated in the pathogenesis of many diseases and disorders, such as cancer, inflammation, and autoimmunity.

In some embodiments, the PD-L1 binding portion is single domain antibody. In some embodiments, the PDL1 binding portion of the fusion blocks or dampens the interaction of PDL1 and PD-1. Exemplary PDL1-targeting single domain sequences are shown below:

28A10:
(SEQ ID NO: 100)

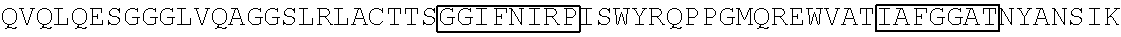

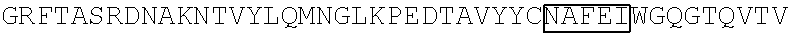

(SEQ ID NO: 101)
CDR1: GGIFNIRP
(SEQ ID NO: 102)
CDR2: IAFGGAT
(SEQ ID NO: 103)
CDR3: NAFEI
28A2:
(SEQ ID NO: 104)

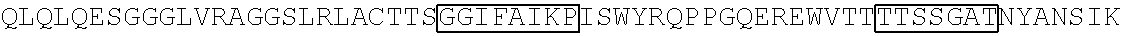

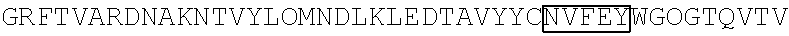

(SEQ ID NO: 105)
CDR1: GGIFAIKP
(SEQ ID NO: 106)
CDR2: TTSSGAT
(SEQ ID NO: 107)
CDR3: NVFEY
B03:
(SEQ ID NO: 108)

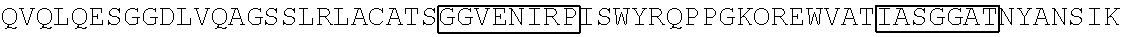

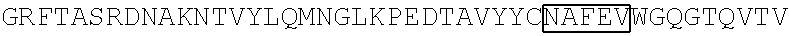

(SEQ ID NO: 109)
CDR1: GGVENIRP
(SEQ ID NO: 110)
CDR2: IASGGAT
(SEQ ID NO: 111)
CDR3: NAFEV
B10:
(SEQ ID NO: 112)

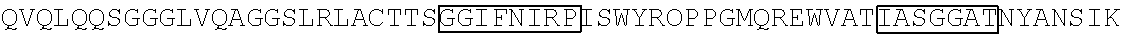

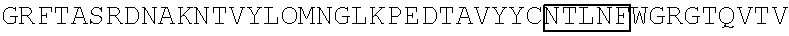

(SEQ ID NO: 101)
CDR1: GGIFNIRP
(SEQ ID NO: 110)
CDR2: IASGGAT
(SEQ ID NO: 113)
CDR3: NTLNF
D02:
(SEQ ID NO: 114)

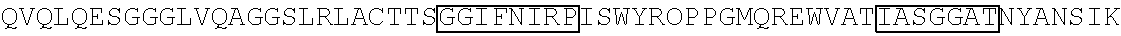

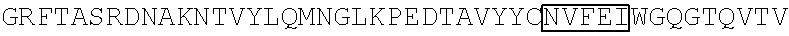

(SEQ ID NO: 101)
CDR1: GGIFNIRP
(SEQ ID NO: 110)
CDR2: IASGGAT
(SEQ ID NO: 115)
CDR3: NVFEI
A03:
(SEQ ID NO: 116)

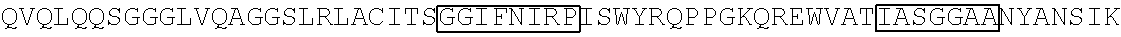

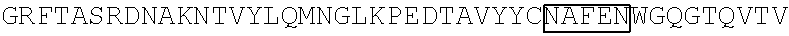

(SEQ ID NO: 101)
CDR1: GGIFNIRP
(SEQ ID NO: 117)
CDR2: IASGGAA
(SEQ ID NO: 118)
CDR3: NAFEN
hz28A2v1
(SEQ ID NO: 119)

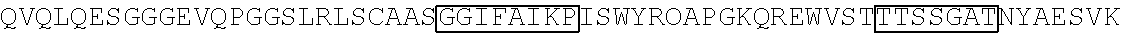

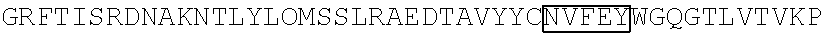

(SEQ ID NO: 105)
CDR1: GGIFAIKP
(SEQ ID NO: 106)
CDR2: TTSSGAT
(SEQ ID NO: 107)
CDR3: NVFEY
hz28A2v1-1
(SEQ ID NO: 120)

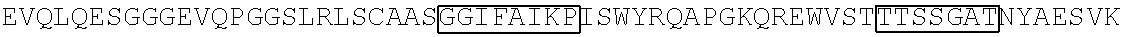

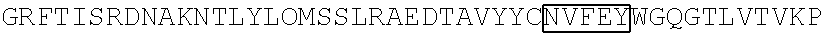

(SEQ ID NO: 105)
CDR1: GGIFAIKP
(SEQ ID NO: 106)
CDR2: TTSSGAT
(SEQ ID NO: 107)
CDR3: NVFEY
hz28A2v2
(SEQ ID NO: 121)

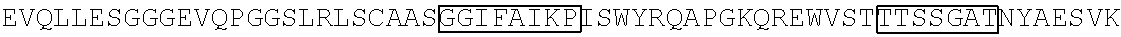

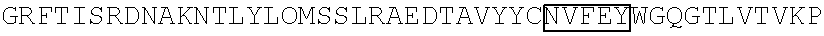

(SEQ ID NO: 105)
CDR1: GGIFAIKP
(SEQ ID NO: 106)
CDR2: TTSSGAT
(SEQ ID NO: 107)
CDR3: NVFEY
hz28A2v3
(SEQ ID NO: 122)

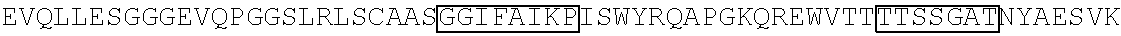

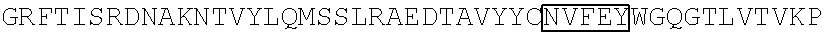

(SEQ ID NO: 105)
CDR1: GGIFAIKP
(SEQ ID NO: 106)
CDR2: TTSSGAT
(SEQ ID NO: 107)
CDR3: NVFEY
hz28A2v4:
(SEQ ID NO: 123)

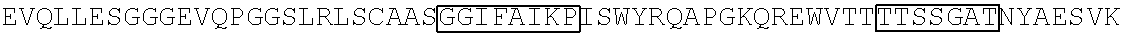

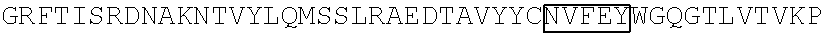

(SEQ ID NO: 105)
CDR1: GGIFAIKP
(SEQ ID NO: 106)
CDR2: TTSSGAT
(SEQ ID NO: 107)
CDR3: NVFEY
hz28A2v5:
(SEQ ID NO: 124)

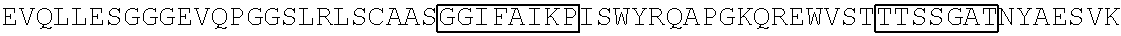

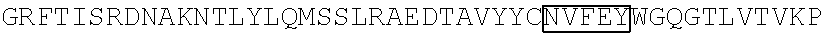

(SEQ ID NO: 105)
CDR1: GGIFAIKP
(SEQ ID NO: 106)
CDR2: TTSSGAT
(SEQ ID NO: 107)
CDR3: NVFEY

In other embodiments, the PD-L1 binding portion is derived from the extracellular domain of PD-1 containing at least the IgV domain as shown below:

(SEQ ID NO: 125)
PTFSPALLVVTEGDNATFTCSFSNTSESFVLNWYRMSPSNQTDKLAAFPE
DRSQPGQDCRFRVTQLPNGRDFHMSVVRARRNDSGTYLCGAISLAPKAQI
KESLRAELRVT

In some embodiments, the PDL1 binding domain comprises or is derived from a known anti-PDL1 antibody sequence or antigen-binding fragment thereof. In some embodiments, the PDL1 binding domain comprises or is derived from an antibody sequence disclosed in PCT Publication No. WO 2016/149201, the contents of which are hereby incorporated by reference in their entirety.

In some embodiments, the PDL1 binding domain comprises or is derived from an antibody sequence or antigen-binding fragment thereof that includes a combination of a variable heavy chain (VH) sequence and a variable light chain (VL) sequence selected from the group consisting of:

VH Sequences:
(SEQ ID NO: 126)
QVQLVQSGAEVKKPGASVKVSCKASGYTFTDYGFSWVRQAPGQGLEWMGW
ITAYNGNTNYAQKLQGRVTMTTDTSTSTVYMELRSLRSDDTAVYYCARDY
FYGMDVWGQGTTVTVSS
(SEQ ID NO: 127)
QVQLVQSGAEVKKPGSSVKVSCKTSGDTFSTYAISWVRQAPGQGLEWMGG
IIPIFGKAHYAQKFQGRVTITADESTSTAYMELSSLRSEDTAVYFCARKF
HFVSGSPFGMDVWGQGTTVTVSS
(SEQ ID NO: 128)
QVQLVQSGAEVKKPGASVKVSCKASGYTFTSYDVHWVRQAPGQRLEWMGW
LHADTGITKFSQKFQGRVTITRDTSASTAYMELSSLRSEDTAVYYCARER
IQLWFDYWGQGT
(SEQ ID NO: 129)
QVQLVQSGAEVKKPGSSVKVSCKVSGGIFSTYAINWVRQAPGQGLEWMGG
IIPIFGTANHAQKFQGRVTITADESTSTAYMELSSLRSEDTAVYYCARDQ
GIAAALFDYWGQGTLVTVSS
(SEQ ID NO: 130)
EVQLVESGGGLVQPGRSLRLSCAVSGFTFDDYVVHWVRQAPGKGLEWVSG
NSGNIGYADSVKGRFTISRDNAKNSLYLQMNSLRAEDTALYYCAVPFDYW
GQGTLVTVSS
(SEQ ID NO: 131)
QVQLVQSGAEVKKPGSSVKVSCKTSGDTFSSYAISWVRQAPGQGLEWMGG
IIPIFGRAHYAQKFQGRVTITADESTSTAYMELSSLRSEDTAVYFCARKF
HFVSGSPFGMDVWGQGTTVTVSS
(SEQ ID NO: 132)
QVQLVQSGAEVKKPGSSVKVSCKTSGGTFSSYAISWVRQAPGQGLEWMGG
IIPIFGKAHYAQKFQGRVTITADESTTTAYMELSSLRSEDTAVYYCARKY
DYVSGSPFGMDVWGQGTTVTVSS
(SEQ ID NO: 133)
QVQLVQSGAEVKKPGSSVKVSCKASGGTFSSYAINWVRQAPGQGLEWMGG
IIPIFGSANYAQKFQDRVTITADESTSAAYMELSSLRSEDTAVYYCARDS
SGWSRYYMDVWGQGTTVTVSS
(SEQ ID NO: 134)
QVQLVQSGAEVKEPGSSVKVSCKASGGTFNSYAISWVRQAPGQGLEWMGG
IIPLEGIAHYAQKFQGRVTITADESTNTAYMDLSSLRSEDTAVYYCARKY
SYVSGSPFGMDVWGQGTTVTVSS
(SEQ ID NO: 135)
EVQLVESGGGLVQPGRSLRLSCAASGITEDDYGMHWVRQAPGKGLEWVSG
ISWNRGRIEYADSVKGRFTISRDNAKNSLYLQMNSLRAEDTALYYCAKGR
FRYFDWFLDYWGQGTLVTVSS
(SEQ ID NO: 136)
QMQLVQSGGGLVQPGGSLRLSCAASGFTFSSYWMSWVRQAPGKGLEWVAN
IKQDGSEKYYVDSVKGRFTISRDNAKNSLYLQMNSLRAEDTAVYYCARDY
FWSGFSAFDIWGKGTLVTVS
VL Sequences:
(SEQ ID NO: 137)
EIVLTQSPATLSLSPGERATLSCRASQSVSSYLVWYQQKPGQAPRLLIYD
ASNRATGIPARFSGSGSGTDFTLTISSLEPEDFAVYYCQQRSNWPRTFGQ
GTKVEIK
(SEQ ID NO: 138)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPTFGQG
TKVEIK
(SEQ ID NO: 139)
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPYTFGQ
GTKLEIK
(SEQ ID NO: 140)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK
(SEQ ID NO: 141)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPFGGG
TKVEIK
(SEQ ID NO: 142)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPTFGQG
TRLEIK
(SEQ ID NO: 143)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPFTFGP
GTKVDIK
(SEQ ID NO: 144)
DIVMTQSPSTLSASVGDRVTITCRASQGISSWLAWYQQKPGRAPKVLIYK
ASTLESGVPSRFSGSGSGTDFTLTISSLQPEDFATYYCQQSYSTPWTFGQ
GTKLEIK

In some embodiments, the PDL1 binding domain comprises or is derived from an antibody sequence or antigen-binding fragment thereof that includes a combination of a VH sequence and a VL sequence selected from the group consisting of:

VH Sequence:
(SEQ ID NO: 145)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
VL Sequence:
(SEQ ID NO: 146)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 147)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA
(SEQ ID NO: 148)
EVQLVESGGGLVQPGGSLRLSCAASGFTFSGSWIHWVRQAPGKGLEWVAW
ILPYGGSSYYADSVKGRFTISADTSKNTAYLQMNSLRAEDTAVYYCARRH
WPGGFDYWGQGTLVTVSA
VL Sequences:
(SEQ ID NO: 149)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR
(SEQ ID NO: 150)
DIQMTQSPSSLSASVGDRVTITCRASQDVSTAVAWYQQKPGKAPKLLIYS
ASFLYSGVPSRFSGSGSGTDFTLTISSLQPEDFATYYCQQYYNVPWTFGQ
GTKVEIKR
(SEQ ID NO: 151)
DIQMTQSPSSLSASVGDRVTITCRASQDVSTAVAWYQQKPGKAPKLLIYS
ASFLYSGVPSRFSGSGSGTDFTLTISSLQPEDFATYYCQQYYAPPWTFGQ
GTKVEIKR
(SEQ ID NO: 152)
DIQMTQSPSSLSASVGDRVTITCRASQDVSTAVAWYQQKPGKAPKLLIYS
ASFLYSGVPSRFSGSGSGTDFTLTISSLQPEDFATYYCQQYYTVPWTFGQ
GTKVEIKR
(SEQ ID NO: 153)
DIQMTQSPSSLSASVGDRVTITCRASQVINTFLAWYQQKPGKAPKLLIYS
ASTLASGVPSRFSGSGSGTDFTLTISSLQPEDFATYYCQQYYTVPRTFGQ
GTKVEIKR
(SEQ ID NO: 154)
DIQMTQSPSSLSASVGDRVTITCRASQDVSTAVAWYQQKPGKAPKLLIYS
ASFLYSGVPSRFSGSGSGTDFTLTISSLQPEDFATYYCQQGYGVPRTFGQ
GTKVEIKR
(SEQ ID NO: 155)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLFTPPTFGQ
GTKVEIKR
(SEQ ID NO: 156)
DIQMTQSPSSLSASVGDRVTITCRASQDVSTAVAWYQQKPGKAPKLLIYS
ASFLYSGVPSRFSGSGSGTDFTLTISSLQPEDFATYYCQQYFITPTTFGQ
GTKVEIKR
(SEQ ID NO: 157)
DIQMTQSPSSLSASVGDRVTITCRASQDVSTAVAWYQQKPGKAPKLLIYS
ASFLYSGVPSRFSGSGSGTDFTLTISSLQPEDFATYYCQQYYYTPPTFGQ
GTKVEIKR
(SEQ ID NO: 158)
DIQMTQSPSSLSASVGDRVTITCRASQDVSTAVAWYQQKPGKAPKLLIYS
ASFLYSGVPSRFSGSGSGTDFTLTISSLQPEDFATYYCQQFFYTPPTFGQ
GTKVEIKR
(SEQ ID NO: 159)
DIQMTQSPSSLSASVGDRVTITCRASQDVSTAVAWYQQKPGKAPKLLIYS
ASFLYSGVPSRFSGSGSGTDFTLTISSLQPEDFATYYCQQSLFTPPTFGQ
GTKVEIKR
(SEQ ID NO: 160)
DIQMTQSPSSLSASVGDRVTITCRASQDVSTAVAWYQQKPGKAPKLLIYS
ASFLYSGVPSRFSGSGSGTDFTLTISSLQPEDFATYYCQQSLYTPPTFGQ
GTKVEIKR
(SEQ ID NO: 161)
DIQMTQSPSSLSASVGDRVTITCRASQDVSTAVAWYQQKPGKAPKLLIYS
ASFLYSGVPSRFSGSGSGTDFTLTISSLQPEDFATYYCQQSWYHPPTFGQ
GTKVEIKR
(SEQ ID NO: 162)
DIQMTQSPSSLSASVGDRVTITCRASQDVSTAVAWYQQKPGKAPKLLIYS
ASFLYSGVPSRFSGSGSGTDFTLTISSLQPEDFATYYCQQYFYIPPTFGQ
GTKVEIKR
(SEQ ID NO: 163)
DIQMTQSPSSLSASVGDRVTITCRASQDVSTAVAWYQQKPGKAPKLLIYS
ASFLYSGVPSRFSGSGSGTDFTLTISSLQPEDFATYYCQQYWYTPTTFGQ
GTKVEIKR
(SEQ ID NO: 164)
DIQMTQSPSSLSASVGDRVTITCRASQDVSTAVAWYQQKPGKAPKLLIYS
ASFLYSGVPSRFSGSGSGTDFTLTISSLQPEDFATYYCQQSYFIPPTFGQ
GTKVEIKR

In some embodiments, the PDL1 binding domain comprises or is derived from an antibody sequence or antigen-binding fragment thereof that includes a combination of a VH sequence and a VL sequence selected from the group consisting of.

VH Sequences:
(SEQ ID NO: 165)
METGLRWLLLVAVLKGVQCLSVEESGGRLVTPGTPLTLTCTASGFTITNY
HMFWVRQAPGKGLEWIGVITSSGIGSSSTTYYATWAKGRFTISKTSTTVN
LRITSPTTEDTATYFCARDYFTNTYYALDIWGPGTLVTVSS
(SEQ ID NO: 166)
QVQLVQSGAEVKKPGSSVKVSCKTSGDTFSTYAISWVRQAPGQGLEWMGG
IIPIFGKAHYAQKFQGRVTITADESTSTAYMELSSLRSEDTAVYFCARKF
HFVSGSPFGMDVWGQGTTVTVSS
(SEQ ID NO: 167)
QVQLVQSGAEVKKPGASVKVSCKASGYTFTSYDVHWVRQAPGQRLEWMGW
LHADTGITKFSQKFQGRVTITRDTSASTAYMELSSLRSEDTAVYYCARER
IQLWFDYWGQGTLVTVSS
(SEQ ID NO: 168)
QVQLVQSGAEVKKPGSSVKVSCKVSGGIFSTYAINWVRQAPGQGLEWMGG
IIPIFGTANHAQKFQGRVTITADESTSTAYMELSSLRSEDTAVYYCARDQ
GIAAALFDYWGQGTLVTVSS
(SEQ ID NO: 169)
EVQLVESGGGLVQPGRSLRLSCAVSGFTFDDYVVHWVRQAPGKGLEWVSG
ISGNSGNIGYADSVKGRFTISRDNAKNSLYLQMNSLRAEDTALYYCAVPF
DYWGQGTLVTVSS
(SEQ ID NO: 170)
QVQLVQSGAEVKKPGSSVKVSCKTSGDTFSSYAISWVRQAPGQGLEWMGG
IIPIFGRAHYAQKFQGRVTITADESTSTAYMELSSLRSEDTAVYFCARKF
HFVSGSPFGMDVWGQGTTVTVSS
(SEQ ID NO: 171)
QVQLVQSGAEVKKPGSSVKVSCKTSGGTFSSYAISWVRQAPGQGLEWMGG
IIPIFGKAHYAQKFQGRVTITADESTTTAYMELSSLRSEDTAVYYCARKY
DYVSGSPFGMDVWGQGTTVTVSS
(SEQ ID NO: 172)
QVQLVQSGAEVKKPGSSVKVSCKASGGTFSSYAINWVRQAPGQGLEWMGG
IIPIFGSANYAQKFQDRVTITADESTSAAYMELSSLRSEDTAVYYCARDS
SGWSRYYMDVWGQGTTVTVSS
(SEQ ID NO: 173)
QVQLVQSGAEVKEPGSSVKVSCKASGGTFNSYAISWVRQAPGQGLEWMGG
IIPLFGIAHYAQKFQGRVTITADESTNTAYMDLSSLRSEDTAVYYCARKY
SYVSGSPFGMDVWGQGTTVTVSS
(SEQ ID NO: 174)
EVQLVESGGGLVQPGRSLRLSCAASGITEDDYGMHWVRQAPGKGLEWVSG
ISWNRGRIEYADSVKGRFTISRDNAKNSLYLQMNSLRAEDTALYYCAKGR
FRYFDWFLDYWGQGTLVTVSS
VL Sequences:
(SEQ ID NO: 175)
MDTRAPTQLLGLLLLWLPGARCALVMTQTPSSTSTAVGGTVTIKCQASQS
ISVYLAWYQQKPGQPPKLLIYSASTLASGVPSRFKGSRSGTEYTLTISGV
QREDAATYYCLGSAGS
(SEQ ID NO: 176)
EIVLTQSPATLSLSPGERATLSCRASQSVSSYLVWYQQKPGQAPRLLIYD
ASNRATGIPARFSGSGSGTDFTLTISSLEPEDFAVYYCQQRSNWPRTFGQ
GTKVEIK
(SEQ ID NO: 177)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPTFGQG
TKVEIK
(SEQ ID NO: 178)
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPYTFGQ
GTKLEIK
(SEQ ID NO: 179)
EIVLTQSPGTLSLSPGERATLSCRASQSVSSSYLAWYQQKPGQAPRLLIY
GASSRATGIPDRFSGSGSGTDETLTISRLEPEDFAVYYCQQYGSSPWTFG
QGTKVEIK
(SEQ ID NO: 180)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPFGGG
TKVEIK
(SEQ ID NO: 181)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPTFGQG
TRLEIK
(SEQ ID NO: 182)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPFTFG
PGTKVDIK

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 183)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSS
VL Sequences:
(SEQ ID NO: 184)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 185)
EVKLQESGPSLVKPSQTLSLTCSVTGYSITSDYWNWIRKFPGNKLEYVGYISYTGSTYYNPSLK
SRISITRDTSKNQYYLQLNSVTSEDTATYYCARYGGWLSPFDYWGQGTTLTVSS
(SEQ ID NO: 186)
EVQLQESGPGLVAPSQSLSITCTVSGFSLTTYSINWIRQPPGKGLEWLGVMWAGGGTNSNSVLK
SRLIISKDNSKSQVFLKMNSLQTDDTARYYCARYYGNSPYYAIDYWGQGTSVTVSS
(SEQ ID NO: 187)
EVKLQESGPSLVKPSQTLSLTCSVTGYSIISDYWNWIRKFPGNKLEYLGYISYTGSTYYNPSLK
SRISITRDTSKNQYYLQLNSVTTEDTATYYCARRGGWLLPFDYWGQGTTLTVSS
(SEQ ID NO: 188)
EVKLQESGPSLVKPGASVKLSCKASGYTFTSYDINWVKQRPGQGLEWIGWIFPRDNNTKYNENF
KGKATLTVDTSSTTAYMELHSLTSEDSAVYFCTKENWVGDFDYWGQGTTLTLSS
(SEQ ID NO: 189)
EVQLQQSGPDLVTPGASVRISCQASGYTFPDYYMNWVKQSHGKSLEWIGDIDPNYGGTTYNQKF
KGKAILTVDRSSSTAYMELRSLTSEDSAVYYCARGALTDWGQGTSLTVSS
(SEQ ID NO: 190)
EIVLTQSPATLSLSPGERATLSCRASSSVSYIYWFQQKPGQSPRPLIYAAFNRATGIPARFSGS
GSGTDYTLTISSLEPEDFAVYYCQQWSNNPLTFGQGTKVEIK
(SEQ ID NO: 191)
QVQLVQSGAEVKKPGASVKVSCKASGYTFPDYYMNWVRQAPGQGLEWMGDIDPNYGGTNYAQKF
QGRVTMTRDTSISTAYMELSRLRSDDTAVYYCARGALTDWGQGTMVTVSS
(SEQ ID NO: 192)
QVQLVQSGAEVKKPGASVKVSCKASGYTFPDYYMNWVRQAPGQSLEWMGDIDPNYGGTNYNQKF
QGRVTMTRDTSISTAYMELSRLRSDDTAVYYCARGALTDWGQGTMVTVSS
(SEQ ID NO: 193)
EVQLVQSGAEVKKPGASVKVSCKASGYTFPDYYMNWVRQAPGQSLEWMGDIDPNYGGTNYNQKF
QGRVTMTVDRSSSTAYMELSRLRSDDTAVYYCARGALTDWGQGTMVTVSS
(SEQ ID NO: 194)
EVQLVESGGGLVQPGRSLRLSCTASGYTFPDYYMNWVRQAPGKGLEWVGDIDPNYGGTTYAASV
KGRFTISVDRSKSIAYLQMSSLKTEDTAVYYCTRGALTDWGQGTMVTVSS
(SEQ ID NO: 195)
EVQLVESGGGLVQPGRSLRLSCTASGYTFPDYYMNWVRQAPGKGLEWVGDIDPNYGGTTYNASV
KGRFTISVDRSKSIAYLQMSSLKTEDTAVYYCARGALTDWGQGTMVTVSS
VL Sequences:
(SEQ ID NO: 196)
DIVMTQSHKLMSTSVGDRVSITCKASQDVGTAVAWYQQKPGQSPKLLIYWASTRHTGVPDRFTG
SGSGTDFTLTISNVQSEDLADYFCQQDSSYPLTFGAGTKVELK
(SEQ ID NO: 197)
DIVTTQSHKLMSTSVGDRVSITCKASQDVGTAVAWYQQKPGQSPKLLIYWASTRHTGVPDRFTG
SGSGTDFTLTISNVQSEDLADYFCQQDSSYPLTFGAGTKVELK
(SEQ ID NO: 198)
DIVMTQSPSSLAVSVGEKVSMGCKSSQSLLYSSNQKNSLAWYQQKPGQSPKLLIDWASTRESGV
PDRFTGSGSGTDFTLTISSVKAEDLAVYYCQQYYGYPLTFGAGTKLELK
(SEQ ID NO: 199)
DIVMTQSPAIMSASPGEKVTMTCSASSSIRYMHWYQQKPGTSPKRWISDTSKLTSGVPARFSGS
GSGTSYALTISSMEAEDAATYYCHQRSSYPWTFGGGTKLEIK
(SEQ ID NO: 200)
QIVLSQSPAILSASPGEKVTMTCRASSSVSYIYWFQQKPGSSPKPWIYATENLASGVPARFSGS
GSGTSYSLTISRVETEDAATYYCQQWSNNPLTFGAGTKLELK
(SEQ ID NO: 201)
EIVLTQSPATLSLSPGERATLSCRASSSVSYIYWFQQKPGQAPRLLIYAAFNRATGIPARFSGS
GSGTDYTLTISSLEPEDFAVYYCQQWSNNPLTFGQGTKVEIK
(SEQ ID NO: 202)
QIVLTQSPATLSLSPGERATLSCRASSSVSYIYWFQQKPGQSPRPLIYATENLASGIPARFSGS
GSGTSYTLTISRLEPEDFAVYYCQQWSNNPLTFGQGTKVEIK
(SEQ ID NO: 203)
DIQLTQSPSSLSASVGDRVTITCRASSGVSYIYWFQQKPGKAPKLLIYAAFNLASGVPSRFSGS
GSGTEYTLTISSLQPEDFATYYCQQWSNNPLTFGQGTKVEIK
(SEQ ID NO: 204)
DIQLTQSPSSLSASVGDRVTITCRASSGVSYIYWFQQKPGKAPKPLIYAAFNLASGVPSRFSGS
GSGTEYTLTISSLQPEDFATYYCQQWSNNPLTFGQGTKVEIK
(SEQ ID NO: 205)
DIQLTQSPSILSASVGDRVTITCRASSSVSYIYWFQQKPGKAPKPLIYATENLASGVPSRFSGS
GSGTSYTLTISSLQPEDFATYYCQQWSNNPLTFGQGTKVEIK

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 206)
QVQLVQSGAEVKKPGASVKVSCKASGYTFTSYGISWVRQAPGQGLEWMGWISAYNGNTNYAQKL
QGRVTMTTDTSTSTAYMELRSLRSDDTAVYYCARALPSGTILVGGWEDPWGQGTLVTVSS
(SEQ ID NO: 207)
EVQLVQSGGGVVQPGRSLRLSCAASGFTFSSYALSWVRQAPGKGLEWVSAISGGGGSTYYADSV
KGRFTISRDNSKNTLYLQMNSLRAEDTAVYYCAKDVFPETFSMNYGMDVWGQGTLVTVSS
(SEQ ID NO: 208)
QVQLVQSGGGVVQPGGSLRLSCAASGFTEDDYAMHWVRQAPGKGLEWVSLISGDGGSTYYADSV
KGRFTISRDNSKNSLYLQMNSLRTEDTALYYCAKVLLPCSSTSCYGSVGAFDIWGQGTTVTVSS
(SEQ ID NO: 209)
QVQLVQSGGSVVRPGESLRLSCVASGFIFDNYDMSWVRQVPGKGLEWVSRVNWNGGSTTYADAV
KGRFTISRDNTKNSLYLQMNNLRAEDTAVYYCVREFVGAYDLWGQGTTVTVSS
(SEQ ID NO: 210)
QVQLVQSGAEVKKPGATVKVSCKVFGDTFRGLYIHWVRQAPGQGLEWMGGIIPIFGTANYAQKF
QGRVTITTDESTSTAYMELSSLRSEDTAVYYCASGLRWGIWGWFDPWGQGTLVTVSS
(SEQ ID NO: 211)
EVQLVQSGAELKKPGSSVKVSCKAFGGTFSDNAISWVRQAPGQGPEWMGGIIPIFGKPNYAQKF
QGRVTITADESTSTAYMVLSSLRSEDTAVYYCARTMVRGFLGVMDVWGQGTTVTVSS
(SEQ ID NO: 212)
QVQLVQSGGGLVQPGGSLRLSCAASGFTFSSYAMSWVRQAPGKGLEWVSAISGSGGSTYYADSV
KGRFTISRDNSKNTLYLQMNSLRAEDTAVYYCAKDQFVTIFGVPRYGMDVWGQGTTVTVSS
(SEQ ID NO: 213)
QVQLVQSGAEVKKPGSSVKVSCKASGGTFSSYAISWVRQAPGQGLEWMGGIIPIFGTANYAQKF
QGRVTITADKSTSTAYMELSSLRSEDTAVYYCARGRQMFGAGIDFWGPGTLVTVSS
(SEQ ID NO: 214)
EVQLVESGAEVKKPGSSVKVSCKVSGGTFGTYALNWVRQAPGQGLEWMGRIVPLIGLVNYAHNF
EGRISITADKSTGTAYMELSNLRSDDTAVYYCAREVYGGNSDYWGQGTLVTVSS
(SEQ ID NO: 215)
QVQLVQSGGEVKKPGASVKVSCKASGYTLSSHGITWVRQAPGQGLEWMGWISAHNGHASNAQKV
EDRVTMTTDTSTNTAYMELRSLTADDTAVYYCARVHAALYYGMDVWGQGTLVTVSS
(SEQ ID NO: 216)
QVQLQESGGGVVQPGRSLRLSCSASGFTFSRHGMHWVRQAPGKGLEWVAVISHDGSVKYYADSM
KGRESISRDNSNNTLYLQMDSLRADDTAVYYCARGLSYQVSGWFDPWGQGTLVTVSS
(SEQ ID NO: 217)
NEMLTQPHSVSESPGKTVTISCTRSSGSIASNYVQWYQQRPGSSPTTVIYEDNQRPSGVPDRFS
GSIDTSSNSASLTISGLKTKDEADYYCQSYDGITVIFGGGTKLTVL
(SEQ ID NO: 218)
NEMLTQPHSVSGSPGKTVTLPCTRSSGSIASHYVQWYQQRPGSAPTTVIYEDNKRPSGVPDRFS
GSIDSSSNSASLSISGLKTEDEADYYCQSYDSSNRWVFGGGTKLTVL
(SEQ ID NO: 219)
LPVLTQPASLSASPGASASLTCTLRSGLNVGSYRIYWYQQKPGSRPQYLLNYKSDSNKQQASGV
PSRFSGSKDASANAGILLISGLQSEDEADYYCMIWYSSAVVFGGGTKLTVL
VL Sequences:
(SEQ ID NO: 220)
NEMLTQPHSVSESPGKTVTISCTRSSGNIASNYVQWYQQRPGSAPTTVIYEDNQRPSGVPDRFS
GSIDSSSNSASLTISGLKTEDEADYYCQSYDSSNLWVFGGGTKLTVL
(SEQ ID NO: 221)
SSELTQDPAVSVALGQTVRITCQGDSLRSYYASWYQQKPGQAPVLVIYGKNNRPSGIPDRFSGS
SSGNTASLTITGAQAEDEADYYCNSRDSSGNHYVFGTGTKVTVL
(SEQ ID NO: 222)
LPVLTQAPSVSVAPGKTARITCGGSDIGRKSVHWYQQKPGQAPALVIYSDRDRPSGISERFSGS
NSGNTATLTISRVEAGDEADYYCQVWDNNSDHYVFGAGTELIVL
(SEQ ID NO: 223)
QSALTQPASVSGSPGQSITISCTGTSSDVGGYNYVSWYQQHPGKAPKLMIYDVSNRPSGVSNRF
SGSKSGNTASLTISGLQAEDEADYYCSSYTSSTLPFGGGTKLTVL
(SEQ ID NO: 224)
EIVLTQSPATLSLSPGERATLSCRASQSIGNSLAWYQQKPGQAPRLLMYGASSRATGIPDRFSG
SGAGTDFTLTISSLEPEDFATYYCQQHTIPTFSFGPGTKVEVK
(SEQ ID NO: 225)
DIVMTQTPSFLSASIGDRVTITCRASQGIGSYLAWYQQRPGEAPKLLIYAASTLQSGVPSRFSG
SGSGTDFTLTISNLQPEDFATYYCQQLNNYPITFGQGTRLEIK
(SEQ ID NO: 226)
QSALTQPPSVSVSPGQTANIPCSGDKLGNKYAYWYQQKPGQSPVLLIYQDIKRPSRIPERFSGS
NSADTATLTISGTQAMDEADYYCQTWDNSVVFGGGTKLTVL
(SEQ ID NO: 227)
NFMLTQPHSVSESPGKTVTISCTRSSGSIDSNYVQWYQQRPGSAPTTVIYEDNQRPSGVPDRFS
GSIDSSSNSASLTISGLKTEDEADYYCQSYDSNNRHVIFGGGTKLTVL
(SEQ ID NO: 228)
NEMLTQPHSVSESPGKTVTISCTRSSGNIGTNYVQWYQQRPGSAPVALIYEDYRRPSGVPDRFS
GSIDSSSNSASLIISGLKPEDEADYYCQSYHSSGWEFGGGTKLTVL
(SEQ ID NO: 229)
QSVLTQPPSVSVAPGQTARITCGGNNIGSKGVHWYQQKPGQAPVLVVYDDSDRPSGIPERFSGS
NSGNTATLTISRVEAGDEADYYCQVWDSSSDHWVEGGGTKLTVL
(SEQ ID NO: 230)
NEMLTQPHSVSESPGKTVTISCTRSSGSIASNYVQWYQQRPGSAPTTVIYEDNQRPSGVPDRFS
GSIDSSSNSASLTISGLKTEDEADYYCQSYDSTTPSVFGGGTKLTVL
(SEQ ID NO: 231)
QVQLVQSGAEVKKPGASVKVSCKASGYTFTSYGISWVRQAPGQGLEWMGWTSPHNGLTAFAQIL
EGRVTMTTDTSTNTAYMELRNLTFDDTAVYFCAKVHPVFSYALDVWGQGTLVTVSS
(SEQ ID NO: 232)
EVQLVESGAEVMNPGSSVRVSCRGSGGDFSTYAFSWVRQAPGQGLEWMGRIIPILGIANYAQKF
QGRVTITADKSTSTAYMELSSLRSDDTAVYYCARDGYGSDPVLWGQGTLVTVSS
(SEQ ID NO: 233)
EVQLVQSGAEVKKPGASVKVSCKASGYTFTNYGISWVRQAPGQGLEWMGWISAYNGNTNYAQKV
QGRVTMTTDTSTSTGYMELRSLRSDDTAVYYCARGDFRKPFDYWGQGTLVTVSS

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 234)
EVQLVQSGPELKKPGASVKMSCKASGYTFTSYVMHWVKQAPGQRLEWIGY
VNPENDGTKYNEMFKGRATLTSDKSTSTAYMELSSLRSEDSAVYYCARQA
WGYPWGQGTLVTVSS
(SEQ ID NO: 235)
EVQLVOSGAEVKKPGASVKMSCKASGYTFTSYVMHWVKQAPGQRLEWIGY
VNPENDGTKYNEMFKGRATLTSDKSTSTAYMELSSLRSEDTAVYYCARQA
WGYPWGQGTLVTVSS
(SEQ ID NO: 236)
EVQLVQSGAEVKKPGASVKMSCKASGYTFTSYVMHWVRQAPGQRLEWIGY
VNPENDGTKYNEMFKGRATLTSDKSTSTAYMELSSLRSEDTAVYYCARQA
WGYPWGQGTLVTVSS
(SEQ ID NO: 237)
EVQLVQSGAEVKKPGASVKVSCKASGYTFTSYVMHWVRQAPGQRLEWIGY
VNPENDGTKYNEMFKGRATLTSDKSTSTAYMELSSLRSEDTAVYYCARQA
WGYPWGQGTLVTVSS
(SEQ ID NO: 238)
EVQLVQSGAEVKKPGASVKVSCKASGYTFTSYVMHWVRQAPGQRLEWIGY
VNPENDGTKYNEMFKGRATITSDKSTSTAYMELSSLRSEDTAVYYCARQA
WGYPWGQGTLVTVSS
VL Sequences:
(SEQ ID NO: 239)
DIVLTQSPASLALSPGERATLSCRATESVEYYGTSLVQWYQQKPGQPPKL
LIYAASSVDSGVPSRFSGSGSGTDFTLTINSLEEEDAAMYFCQQSRRVPY
TFGQGTKLEIK
(SEQ ID NO: 240)
DIVLTQSPATLSLSPGERATLSCRATESVEYYGTSLVQWYQQKPGQPPKL
LIYAASSVDSGVPSRFSGSGSGTDFTLTINSLEAEDAAMYFCQQSRRVPY
TFGQGTKLEIK
(SEQ ID NO: 241)
EIVLTQSPATLSLSPGERATLSCRATESVEYYGTSLVQWYQQKPGQPPKL
LIYAASSVDSGVPSRFSGSGSGTDFTLTINSLEAEDAAMYFCQQSRRVPY
TFGQGTKLEIK
(SEQ ID NO: 242)
DIVLTQSPATLSLSPGERATLSCRATESVEYYGTSLVQWYQQKPGQPPKL
LIYAASSVDSGVPSRFSGSGSGTDFTLTINSLEAEDAATYFCQQSRRVPY
TFGQGTKLEIK

In some embodiments, the PDL1 binding domain comprises or is derived from an antibody sequence or antigen-binding fragment thereof that includes a combination of a VH sequence and a VL sequence selected from the group consisting of.

VH Sequences:
(SEQ ID NO: 243)
EVQLVQSGAEVKKPGSSVKVSCKASGGTFSSYAISWVRQAPGQGL
EWMGGIIPIFGTANYAQKFQGRVTITADKSTSTAYMELSSLRSED
TAVYYCAREGTIYDSSGYSFDYWGQGTLVTVSS
(SEQ ID NO: 244)
EVQLVQSGAEVKKPGSSVKVSCKASGGTFSSYAISWVRQAPGQGL
EWMGIINPSGGSTSYAQKFQGRVSMTRDTSTSTVYMELSSLTSED
TAVYYCARDLFPHIYGNYYGMDIWGQGTTVTVSS
(SEQ ID NO: 245)
QVQLVQSGAEVKKPGSSVKVSCKASGGTFSSYAISWVRQAPGQGL
EWMGGIIPIFGTANYAQKFQGRVTITADKSTSTAYMELSSLRSED
TAVYYCARLAVPGAFDIWGQGTMVTVSS
(SEQ ID NO: 246)
EVQLVESGGGVVQPGRSLRLSCAASGFTFSSYAMHWVRQAPGKGL
AVISYDGSNKYYADSVKGRFTISRDNSKNTLYLQMNSLRAEDTAV
YYCARGQWLVTELDYWGQGTLVTVSS
(SEQ ID NO: 247)
EVQLVESGSEVEKPGSSVKVSCKASGGTFSDSGISWVRQAPGQGL
EWMGGIIPMFATPYYAQKEQDRVTITADESTSTVYMELSGLRSDD
TAVFYCARDRGRGHLPWYFDLWGRGTLVTVSS
(SEQ ID NO: 248)
EVQLVESGAEVKKPGSSVKVSCKASGGTFSSYAISWVRQAPGQGL
EWMGGIIPIFGTANYAQKFQGRVTITADESTSTAYMELSSLRSED
TAVYYCARAPYYYYYMDVWGQGTTVTVSS
(SEQ ID NO: 249)
EVQLLESGAEVKKPGSSVKVSCKASGGTLSRYALSWVRQAPGQGP
EWVGAIIPIFGTPHYSKKFQDRVIITVDTSTNTAFMELSSLRFED
TALYFCARGHDEYDISGYHRLDYWGQGTLVTVSS
(SEQ ID NO: 250)
QVQLVQSGSELKKPGSSVKVSCKASGYSFSGYYIHWVRQAPGQGL
EWMGWIDPNSGVTNYVRRFQGRVTMTRDTSLSTAYMELSGLTADD
TAVYYCARDENLWQFGYLDYWGQGTLVTVSS
(SEQ ID NO: 251)
QVQLVQSGAEVKKPGSSVKVSCKASGGTFSRYGVHWVRQAPGQGL
EWMGRLIPIVSMTNYAQKFQDRVSITTDKSTGTAYMELRSLTSED
TALYYCASVGQQLPWVFFAWGQGTLVTVSS
(SEQ ID NO: 252)
QVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 253)
EVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 254)
EVQLVQSGGGLVQPGGSLRLSCAASGFTFSDYGMHWVRQPPGKGL
EWLAVISYDGSYKIHADSVQGRFTISRDNAKNSVFLQMNSLKTED
TAVYYCTTDRKWLAWHGMDVWGQGTTVTVSS
(SEQ ID NO: 255)
EVQLVQSGAEVKKPGSSVKVSCKASGGTFSSYAISWVRQAPGQGL
EWMGGIIPIFGTANYAQKFQGRVTITADESTSTAYMELSSLRSED
TAVYYCARDGIVADFQHWGQGTLVTVSS
(SEQ ID NO: 256)
EVQLVESGAEVKKPGASVKVSCKASGDTFSRYGITWVRQAPGRGL
EWMGNIVPFFGATNYAQKEQGRLTITADKSSYTSYMDLSSLRSDD
TAVYYCARDHFYGSGGYFDYWGQGTLVTVSS
(SEQ ID NO: 257)
EVQLLESGAEVKKPGASVKVSCKASGYTFNSYDINWVRQAPGQGL
EWMGGIIPVFGTANYAESFQGRVTMTADHSTSTAYMELNNLRSED
TAVYYCARDRWHYESRPMDVWGQGTTVTVSS
(SEQ ID NO: 258)
EVQLVESGGGLVRPGGSLRLACAASGESFSDYYMTWIRQAPGRGL
EWIAYISDSGQTVHYADSVKGRFTISRDNTKNSLFLQVNTLRAED
TAVYYCAREDLLGYYLQSWGQGTLVTVSS
(SEQ ID NO: 259)
QVQLQQSGPGLVKPSQTLSLTCAISGDSVSSNSAAWNWIRQSPSR
GLEWLGRTYYRSKWYNDYAVSVKSRITINPDTSKNQFSLQLNSVT
PEDTAVYYCARDEPRAVAGSQAYYYYGMDVWGQGTTVTVSS
(SEQ ID NO: 260)
EVQLVQSGAEVKKPGASVKVSCKASGYTFTSYYMHWVRQAPGQGL
EWMGIINPSDGSTSYAQKFQGRVTMTRDTSTSTVHMELSSLRSED
TAVYYCARDLFPHIYGNYYGMDIWGQGTTVTVSS
(SEQ ID NO: 261)
QMQLVQSGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 262)
QVQLVQSGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
VL Sequences:
(SEQ ID NO: 263)
QSVLTQPPSVSAAPGQKVTISCSGNNSNIANNYVSWYQQLPGTAP
KLLIYDNNYRPSGIPDRESGSKSGTSATLDITGLQTGDEADYYCG
VWDGSLTTGVFGGGTKLTVL
(SEQ ID NO: 264)
AIQMTQSPSSLSASVGDRVTITCRASQGISNYLAWYQQKPGKVPK
LLIYAASTLESGVPSRESGSGSGTDFTLTISSLQPEDLATYYCQQ
LHTFPLTFGGGTKVEIK
(SEQ ID NO: 265)
QPVLTQPPSASGSPGQSVTISCTGTSSDVGAYNFVSWYRQHPGKA
PKLMIYEVNKRPSGVPDRFSGSKSGNTASLTVSGLQAEDEADYYC
SSYAGTNSLGIFGTGTKLTVL
(SEQ ID NO: 266)
QSVVTQPPSVSAAPGQKVTISCSGSSSDIGNHYVSWYQQLPGTAP
KLLIYDNNQRPSGIPDRESGSKSGTSATLAITGLQTGDEADYYCG
TWDNSLSPHLLFGGGTKLTVL
(SEQ ID NO: 267)
QSVLTQPPSVSAAPGQKVTISCSGSSSNMGNNYVSWYKQVPGTAP
KLLIYENDKRPSGIPDRESGSKSGTSATLGITGLQTGDEADYYCG
TWDNSLSGFVFASGTKVTVL
(SEQ ID NO: 268)
QSALTQPASVSGSLGQSVTISCTGSSSDVGSYNLVSWYQQHPGKA
PNLMIYDVSKRSGVSNRESGSKSGNTASLTISGLQAEDEADYYCS
SYTGISTVVFGGGTKLTVL
(SEQ ID NO: 269)
QSVLTQPASVSGSPGQSITISCTGTSSDVGSYNLVSWYQQHPGKA
PKLMIYEVSKRPSGVSNRFSGSKSGNTASLTISGLQAEDEADYYC
SSYGGENNLLFGGGTKLTVL
(SEQ ID NO: 270)
DIVMTQSPSSLSASIGDRVTITCRASQRISAYVNWYQQKPGKAPK
VLIYAASSLRSGVPSRESGSGSGTDFTLTISSLQPEDFATYYCQQ
TYSSPWTFGQGTKVEIK
(SEQ ID NO: 271)
QSVLTQPPSASGSPGQSVTISCTGTSSDIGGYDSVSWYQQHPGKA
PKLMIYDVSKRPSGVSNRFSGSKSGNTASLTISGLQAEDEADYYC
SSYTSSSIFFYVFGTGTKVTVL
(SEQ ID NO: 272)
LPVLTQPASVSGSPGQSITISCTGTTSDIGGYDYVSWYQQHPGKA
PKLMIYDVSKRPSGVSNRFSGSKSGNTASLTISGLQAEDEADYYC
SSYTSSSTHVFGTGTKLTVL
(SEQ ID NO: 273)
QSALTQPASVSGSPGQSITISCTGTSSDVGGYNYVSWYQQHPGKA
PKLMIYDVSNRPSGVSNRFSGSKSGNTASLTISGLQAEDEADYYC
SSYRSSTLGPVFGGGTKLTVL
(SEQ ID NO: 274)
QAGLTQPPSVSEAPRQRVTISCSGSSSNIGNNAVNWYQQLPGKAP
KLLIYYDDLLPSGVSDRESGSKSGTSASLAISGLQSEDEADYYCA
AWDDSLNGYVFGTGTKLTVL
(SEQ ID NO: 275)
QSALTQPRSVSGSPGQSVTISCTGTSSDVGGYNYVSWYQQHPGKA
PKLMIYDVSKRPSGVPDRFSGSKSGNTASLTISGLQAEDEADYYC
SSYTSSTTHVFGTGTKVTVL
(SEQ ID NO: 276)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSVWVFGGGTQLTVL
(SEQ ID NO: 277)
QSVLTQPASVSGSPGQSITISCTGTSSDVGGYNYVSWYQQHPGRA
PRLMIYDVSNRPSGVSNRFSGSKSGNTASLTISGLQAEDEGDYYC
SSYTSGGTLGPVFGGGTKLTVL
(SEQ ID NO: 278)
QAGLTQPPSASGTPGQRVTISCSGSSSNIGSNTVNWYQQLPGTAP
KLLIYSNNQRPSGVPDRESGSKSGTSASLAISGLQSEDEADYYCA
AWDDSLNGWVFGGGTKLTVL
(SEQ ID NO: 279)
AIRMTQSPSSLSASVGDRVTITCRASQSISNYLNWYQQRPGKAPN
LLIYAASSLQSGVPSRESGSGSGTDFTLTISSLQPEDFATYYCQQ
TYSTPYTFGQGTKLEIK
(SEQ ID NO: 280)
QSVLTQPASVSGSPGQSITISCTGTSSDVGGYNYVSWYRQHPGKA
PKLMIYDVSYRPSGVSNRFSGSKSGNTASLTISGLQAEDEADYYC
SSYTDSSTRYVFGTGTKLTVL
(SEQ ID NO: 281)
QPVLTQPPSASGTPGQRVAISCSGSRSNIEINSVNWYQQLPGTAP
KLLIYDNNKRPSGIPDRESGSKSGTSATLGITGLQTGDEADYYCG
SWDSSLSADVEGTGTKLTVL
(SEQ ID NO: 282)
QSVLTQPPSVSAAPGKKVTISCSGSSSNIGNNYVSWYQQLPGTAP
KLLIYRNNQRPSGVPDRESGSKSGTSASLAISGLQSEDEADYYCA
TWDDSLNGWVFGGGTKLTVL
(SEQ ID NO: 283)
QSVVTQPPSVSGAPGQRVTISCTGSSSNIGAGYDVHWYQQLPGTA
PKLLIYGNNNRHSGVPDRESGSKSGTSASLAITGLQAEDEAEFFC
GTWDSRLTTYVFGSGTKLTVL
(SEQ ID NO: 284)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSAVVFGGGTKLTVL
(SEQ ID NO: 285)
VIWMTQSPSSLSASVGDRVTITCAASSLQSWYQQKPGKAPKLLIY
EASTLESGVPSRFSGSGSGTEFTLTISSLQPEDFATYYCQQSYST
PYTFGQGTKLEIK
(SEQ ID NO: 286)
QSVVTQPPSVSAAPGQKVTISCSGSSSNIGNNYVSWYQQVPGTAP
KLLIYDNNKRPSGIPDRESGSNSDTSATLGITGLQTGDEADYYCG
TWDSSLSAWVEGGGTKLTVL
(SEQ ID NO: 287)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSAGSVVFGGGTKLTVL
(SEQ ID NO: 288)
SYELMQPPSVSVAPGKTATIACGGENIGRKTVHWYQQKPGQAPVL
VIYYDSDRPSGIPERFSGSNSGNTATLTISRVEAGDEADYYCLVW
DSSSDHRIFGGGTKLTVL
(SEQ ID NO: 289)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL
(SEQ ID NO: 290)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL
(SEQ ID NO: 291)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL

In some embodiments, the PDL1 binding domain comprises or is derived from an antibody sequence or antigen-binding fragment thereof that includes a combination of a heavy chain (HC) and a light chain sequence (LC) selected from the group consisting of:

HC Sequences:
(SEQ ID NO: 292)
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGK
(SEQ ID NO: 293)
QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSSASTKGPSVFPLAPCSRSTSEST
AALGCLVKDYFPEPVTVSWNSGALTSGVHTFPAVLQSSGLYSLSS
VVTVPSSSLGTKTYTCNVDHKPSNTKVDKRVESKYGPPCPPCPAP
EFLGGPSVFLFPPKPKDTLMISRTPEVTCVVVDVSQEDPEVQFNW
YVDGVEVHNAKTKPREEQFNSTYRVVSVLTVLHQDWLNGKEYKCK
VSNKGLPSSIEKTISKAKGQPREPQVYTLPPSQEEMTKNQVSLTC
LVKGFYPSDIAVEWESNGQPENNYKTTPPVLDSDGSFFLYSRLTV
DKSRWQEGNVFSCSVMHEALHNHYTQKSLSLSLGK
LC Sequences:
(SEQ ID NO: 294)
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSENRGEC
(SEQ ID NO: 295)
EIVLTQSPATLSLSPGERATLSCRASQSVSSYLAWYQQKPGQAPR
LLIYDASNRATGIPARESGSGSGTDFTLTISSLEPEDFAVYYCQQ
SSNWPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 296)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTK
(SEQ ID NO: 297)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSS
HC Sequences:
(SEQ ID NO: 298)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTKGPSVEPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYASTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPG
VL Sequences:
(SEQ ID NO: 299)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKR
LC Sequences:
(SEQ ID NO: 300)
DIQMTQSPSSLSASVGDRVTITCRASQDVSTAVAWYQQKPGKAPK
LLIYSASFLYSGVPSRESGSGSGTDFTLTISSLQPEDFATYYCQQ
YLYHPATFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 301)
EVQLVESGGGLVQPGGSLRLSCAASGFTFSRFWMSWVRQAPGKGL
EWVANINQDGTEKYYVDSVKGRFTISRDNAKNSLYLQMNSLRAGD
TAVYYCANTYYDFWSGHFDYWGQGTLVTVSS
(SEQ ID NO: 302)
QEHLVESGGGVVQPGRSLRLSCEASGFTFSNFGMHWVRQAPGKGL
EWVAALWSDGSNKYYADSVKGRVTISRDNSKNTLYLQMNSLRAED
TAVYYCARGRGAPGIPIFGYWGQGTLVTVSS
(SEQ ID NO: 303)
EVQLVESGGGLVKPGGSLRLSCAASGFTFSNAWMSWVRQAPGKGL
EWVGRIKRKTDGGTTDYAAPVKGRFTISRDDSKNTLHLQMNSLKT
EDTAVYYCTTDDIVVVPAVMREYYFGMDVWGQGTTVTVSS
(SEQ ID NO: 304)
QVQLVQSGAEVKKPGASVQVSCKASGYSFTGYYIHWVRQAPGQGL
EWMGWINPNSGTKKYAHKFQGRVTMTRDTSIDTAYMILSSLISDD
TAVYYCARDEDWNFGSWFDSWGQGTLVTVSS
(SEQ ID NO: 305)
QVHLVQSGAEVKKPGASVKVSCKASGYTFTGYYIHWVRQAPGHGL
EWMGWLNPNTGTTKYIQNFQGRVTMTRDTSSSTAYMELTRLRSDD
TAVYYCARDEDWNYGSWFDTWGQGTLVTVSS
(SEQ ID NO: 306)
EVQLVESGGGVVRPGGSLRLSCAASGFTFDDYGMTWVRQAPGRGL
EWVSGIHWHGKRTGYADSVKGRFTISRDNAKKSLYLQMNSLKGED
TALYHCVRGGMSTGDWEDPWGQGTLVIVSS
(SEQ ID NO: 307)
EVQLVESGGGVVRPGGSLRLSCAASGFTEDDYGMTWVRQVPGKGL
EWVSGIHWSGRSTGYADSVKGRFTISRDNAKNSLYLQMNSLRAED
TALYYCARGGMSTGDWEDPWGQGTLVTVSS
(SEQ ID NO: 308)
EVQLVESGGGLVQPGGSLRLSCAASGFTVGSNYMNWVRQAPGKGL
EWVSVIYSGGSTYYADSVKGRFTISRLTSKNTLYLQMSSLRPEDT
AVYYCARGIRGLDVWGQGTTVTVSS
(SEQ ID NO: 309)
EERLVESGGDLVQPGGSLRLSCAASGITVGTNYMNWVRQAPGKGL
EWVSVISSGGNTHYADSVKGRFIMSRQTSKNTLYLQMNSLETEDT
AVYYCARGIRGLDVWGQGTMVTVSS
(SEQ ID NO: 310)
QVQLVQSGAEVKMPGSSVRVSCKASGGIFSSSTISWVRQAPGQGL
EWMGEIIPVFGTVNYAQKFQDRVIFTADESTTTAYMELSSLKSGD
TAVYFCARNWGLGSFYIWGQGTMVTVSS
(SEQ ID NO: 311)
EVQLVESGGDLVHPGRSLRLSCAASGFPFDEYAMHWVRQVPGKGL
EWVSGISWSNNNIGYADSVKGRFTISRDNAKNSLYLQMNSLRPED
TAFYYCAKSGIFDSWGQGTLVTVSS
(SEQ ID NO: 312)
EVQLVESGGGVVQPGRSLRLSCAASGFTFSSYGMHWVRQAPGKGL
EWVTLISYEGRNKYYADSVKGRFTISRDNSKNTLYLQMNSLRAED
TAVYYCAKDRTLYGMDVWGQGTTVTVSS
(SEQ ID NO: 313)
QVTLRESGPALVKTTQTLTLTCTFSGESLSTNRMCVTWIRQPPGK
ALEWLARIDWDGVKYYNTSLKTRLTISKDTSKNQVVLTMTNMDPV
DTATFYCARSTSLTFYYFDYWGQGTLVTVSS
(SEQ ID NO: 314)
EVQLVESGGGLVQPGGSLRLSCAASEFTVGTNHMNWVRQAPGKGL
EWVSVIYSGGNTFYADSVKGRFTISRHTSKNTLYLQMNSLTAEDT
AVYYCARGLGGMDVWGQGTTVTVSS
(SEQ ID NO: 315)
EVQLVESGGGLVQRGESLRLYCAASGFTFSKYWMNWVRQAPGKGL
EWVANIKGDGSEKYYVDSVKGRFTISRDNAKNSLYLQMNSLRAED
TAVYYCARDYWGSGYYFDFWGQGTLVTVSS
(SEQ ID NO: 316)
EVQLVESGGGLVQSGGSLRLSCAASGFTFSSYWMSWVRQAPGKGL
EWVANIKQDGSEKYYVDSVKGRFTISRDNAKNSLYLQMNSLRADD
TAVYYCARDDIVVVPAPMGYYYYYFGMDVWGQGTTVTVSS
(SEQ ID NO: 317)
EVQLVESGGGLVQPGRSLRLSCAASGFTFDDFAMHWVRQAPGKGL
EWVSGISWTGGNMDYANSVKGRFTISREDAKNSLYLQMNSLRAAD
TALYYCVKDIRGIVATGGAFDIWGRGTMVTVSS
(SEQ ID NO: 318)
EVQLVESGGGLVQPGGSLRLSCAASGFTVGTNYMNWVRQAPGKGL
EWISVIYSGGSTFYADSVKGRFTISRQTSQNTLYLQMNSLRPEDT
AVYYCARGIRGFDIWGQGTMVTVSS
(SEQ ID NO: 319)
EVQLVESGGGLVQPGGSLRLSCAASGFTISTNYMNWVRQAPGKGL
EWVAVIYSSGSTYYIDSVKGRFTISRLTSKNTVYLQMSSLNSEDT
AVYYCARGIRGFDIWGQGTMVTVSS
(SEQ ID NO: 320)
EVQLVESGGGLVQPGRSLRLSCAASGFTIDDSAMHWVRQTPGKGL
EWVSGISWKSGSIGYADSVRGRFTISRDNAKNSLYLQMNSLRVED
TALYYCVKDIRGNWNYGGNWEDPWGQGTLVTVSS
(SEQ ID NO: 321)
EVQLVESGGGLVQPGGSLRLSCEASGFTVGVNHMNWVRQAPGKGL
EWVSVIFSSGRTFYGDYVKGRLTIFRQTSQNTVYLQMNSLRSEDT
AIYYCARGIGGLDIWGRGTMVTVSS
(SEQ ID NO: 322)
EVQLVESGGGLVQPGRSLRLSCAASGFTFDDYALHWVRQAPGKGL
EWVSGISWTGGTIDYADSVKGRFTISRDNAKNSLYLQMSSLRTED
TAIYYCTRDIRGNWKYGGWFDPWGQGTLVTVSS
(SEQ ID NO: 323)
QVQLVQSGTEVKKPGASVKVSCKASGYTFTAYYMHWVRQAPGQGL
DWMGWISPNSGFTNYAQKFQGRVTMTRDTSINTFYMELSGLRSDD
TAVYYCAREGSTHHNSFDPWGQGTLVTVSS
(SEQ ID NO: 324)
EVQLVESGGGLVQPGGSLRLSCAASGFTVGTNFMNWVRQAPGKGL
EWVSAIYSGGTANYADSVKGRFTISRDTSRNTLYLQMNSLRTEDT
AVYYCARGGGMDVWGQGTTVTVSS
(SEQ ID NO: 325)
QVQLVQSGAEVKKPGSSVKVSCKASGGTFNTYVLSWVRQAPGQGL
EWMGEIIPILGAANYAQNFQGRVTFTTDESTNTAYMDLSSLRSED
TAVYYCARDRTSGGFDPWGQGTLVTVSS
(SEQ ID NO: 326)
QVQLVQSGAEVEKPGASVKVSCKASGYIFTHYGISWVRQAPGQGL
EWVGWISPYNGYTDYAQKLQGRVTLTTDTSTTTAYMELRNLRSDD
TAMYYCSRGRGPYWSFDLWGRGTLVTVSS
VL Sequences:
(SEQ ID NO: 327)
DIQMTQSPSTLSASVGDRVTITCRASQSISNWLAWYQQKPGKAPK
LLIYKASSLESGVPSRFSGSGSGTEFTLTISSLQPDDFATYYCQQ
YHSYSYTFGQGTKEIK
(SEQ ID NO: 328)
DIQMTQSPSSLSASVGDRVTITCRASQGIRNDLGWYQQKPGKAPK
RLIYTASSLQSGVPSRESGSGSGTEFTLTISSLQPEDFATYYCLQ
HNSYPLTFGGGTKVAIK
(SEQ ID NO: 329)
DIQMTQSPSSLSASVGDRVTITCRTSQGIRNDLGWYQQKPGKAPK
RLIYAASSLQSGVPSRFSGSGSGTEFTLTISSLQPEDFATYYCLQ
HNNYPYTFGQGTKLEIK
(SEQ ID NO: 330)
DIVMTQTPLSSPVTLGQPASISCRSSQTLVHGDGNTYLSWIQQRP
GQPPRLLIYKVSNQFSGVPDRFSGSGAGTDFTLKISRVEAEDVGL
YFCMQATHEPITFGQGTRLEIK
(SEQ ID NO: 331)
DIVMTQTPLSSPVTLGQPASISCRSSPSLVHSDGNTYLSWLQQRP
GQPPRLLIYKISNRFSGVPDRFSGSGAGTDFTLKISRVEAEDVGV
YYCMQATHFPITFGQGTRLEIR
(SEQ ID NO: 332)
DIQMTQSPSSLSASLGDRVTITCRASQSINSYLNWYQQKPGKAPK
LLIYVASSLQSGVPSRFSGSGSGTEFTLTISNLQPEDFATYYCQQ
SYSTPPITFGQGTRLEIK
(SEQ ID NO: 333)
DIQMTQSPSSLSASVGDRVTITCRASQSISSYLNWYQQKPGKAPK
LLIYVASSLQSGVPSRFSGSGSGTDFTLTISSLQPEDFATYYCQQ
SYSTPPITFGQGTRLEIK
(SEQ ID NO: 334)
DIQMTQSPSSLSASVGDRVTITCRASQTINIYLNWYQQKPGRAPR
LLIYAASSLQSGVPSRESGSGSGTDFTLTISSLQPEDFATYYCHQ
SYSTPPITFGQGTRLEIK
(SEQ ID NO: 335)
DIQMTQSPSSLSASVGDRVTITCRASQSMSSYLNWYQQKPGRAPK
LLIFAASSLQSGVPSRESGSGSGTDFTLTISSLQPEDFATYYCQQ
SYSTPPITFGQGTRLEIK
(SEQ ID NO: 336)
EIVLTQSPGTLSLSPGERATLSCRASQSFNFNYLAWYQQKPGQAP
RLLIYGASSRATGIPDRESGSGSGTDFTLTINRLEPEDFGVFYCQ
QYESAPWTFGQGTKVEIK
(SEQ ID NO: 337)
DIQMTQSPSSLSASVGDRVTITCRASQSISSYLNWYQQKPGKLLI
YAASSLQSGVPSRESGGGSGTDFTLTISSLRPEDFATYYCQQSYC
TPPITFGQGTRLEIK
(SEQ ID NO: 338)
DIQMTQSPSSLSASVGDRVTITCRASQSISSYLNWYQQKPGKAPK
LLIYAASSLQSGVPSRESGSGSGTDFTLTISSLQPEDFATYYCQQ
SYSTPPITFGQGTRLEIK
(SEQ ID NO: 339)
DRVTITCRASQVISNYLAWYQQKPGKVPRLLIYAASTLQSGVPSR
FSGSGSGTDFTLTISSLQPEDVATYYCQKYNSAPRTFGQGTKVEI
K
(SEQ ID NO: 340)
DIQMTQSPSSLSASVGDRVTITCRASQNINNYLNWYQQKPGKAPK
LLIYAASSFQNAVPSRESGSGSGTDFTLTISSLQPEDFATYYCQQ
SYNTPLTFGGGTKVEIK
(SEQ ID NO: 341)
DIQMTQSPSSLSASVGDRVTITCRASQGIRNDLGWYQQKPGKAPK
RLIYAASSLQSGVPSRFSGSGSGTEFTLTISSLQPEDFATYYCLQ
HNSYPYTFGQGTKLEIK
(SEQ ID NO: 342)
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTPPITFGQGTRLEIK

In some embodiments, the PDL1 binding domain comprises or is derived from an antibody sequence or antigen-binding fragment thereof that includes a combination of a VH sequence and a VL sequence selected from the group consisting of.

VH Sequences:
(SEQ ID NO: 343)
QSLEESGGRLVKPDETLTITCTVSGIDLSSNGLTWVRQAPGEGLE
WIGTINKDASAYYASWAKGRLTISKPSSTKVDLKITSPTTEDTAT
YFCGRIAFKTGTSIWGPGTLVTVSS
VL Sequences:
(SEQ ID NO: 344)
AIVMTQTPSPVSAAVGGTVTINCQASESVYSNNYLSWFQQKPGQP
PKLLIYLASTLASGVPSRFKGSGSGTQFTLTISGVQCDDAATYYC
IGGKSSSTDGNAFGGGTEVVVR

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 345)
QMQLVQSGAEVKKPGSSVKVSCKASGGTFSSYAISWVRQAPGQGL
EWMGGIIPIFGTANYAQKFQGRVTITADESTSTAYMELSSLRSED
TAVYYCARGNIVATITPLDYWGQGTLVTVSS
(SEQ ID NO: 346)
QPVLTQPPSVSAAPGQKVTISCSGSSSNIANNYVSWYQQLPGTAP
KLLIFANNKRPSGIPDRESGSKSGTSAALDITGLQTGDEADYYCG
TWDSDLRAGVFGGGTKLTVL
(SEQ ID NO: 347)
EVQLVQSGAEVKKPGSSVKVSCKASGGTFSSYAISWVRQAPGQGL
EWMGGIIPIFGTANYAQKFQGRVTITADKSTSTAYMELSSLRSED
TAVYYCAREGTIYDSSGYSFDYWGQGTLVTVSS
(SEQ ID NO: 348)
QVQLVESGGGVVQPGRSLRLSCAASGFTFSSYAMHWVRQAPGKGL
EWVAVISFDGSNKYYADSVRGRFTISRDNSKNTLYLQMNSLRTED
TAVYYCARGWLDRDIDYWGQGTLVTVSS
(SEQ ID NO: 349)
EVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 350)
QVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 351)
EVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 352)
EVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 353)
QVQLVESGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 354)
QVQLVQSGAEVKKPGSSVKVSCKASGGTFSRYGVHWVRQAPGQGL
EWMGRLIPIVSMTNYAQKFQDRVSITTDKSTGTAYMELRSLTSED
TALYYCASVGQQLPWVFFAWGQGTLVTVSS
(SEQ ID NO: 355)
QMQLVQSGGGVVQPGRSLRLSCAASGFTFSSYAMHWVRQAPGKGL
EWVAVISFDGSNKYYADSVRGRFTISRDNSKNTLYLQMNSLRTED
TAVYYCARGWLDRDIDYWGQGTLVTVSS
(SEQ ID NO: 356)
QVQLVQSGGGVVQPGRSLRLSCAASGFTFSSYAMHWVRQAPGKGL
EWVAVISEDGSNKYYADSVRGRFTISRDNSKNTLYLQMNSLRTED
TAVYYCARGWLDRDIDYWGQGTLVTVSS
(SEQ ID NO: 357)
QMQLVQSGAEVKKPGSSVKVSCKASGGTFSSYAYSWVRQAPGQGL
EWMGGIIPSFGTANYAQKFQGRVTITADESTSTAYMELSSLRSED
TAVYYCARGPIVATITPLDYWGQGTLVTVSS
(SEQ ID NO: 358)
QMQLVQSGAEVKKPGSSVKVSCKASGGTFSSYAYSWVRQAPGQGL
EWMGGIIPIFGTANYAQKEQGRVTITADESTSTAYMELSSLRSED
TAVYYCARGPIVATITPLDYWGQGTLVTVSS
(SEQ ID NO: 359)
QMQLVQSGAEVKKPGSSVKVSCKASGGTFSSYAYSWVRQAPGQGL
EWMGGIIPSFGTANYAQKFQGRVTITADESTSTAYMELSSLRSED
TAVYYCARGPIVATITPLDYWGQGTLVTVSS
(SEQ ID NO: 360)
QMQLVQSGAEVKKPGSSVKVSCKASGGTFSSYAISWVRQAPGQGL
EWMGGIIPAFGTANYAQKFQGRVTITADESTSTAYMELSSLRSED
TAVYYCARGPIVATITPLDYWGQGTLVTVSS
VL Sequences:
(SEQ ID NO: 361)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL
(SEQ ID NO: 362)
AIRMTQSPSSLSASVGDRVTITCRASQSISSYLNWYQQKPGKAPK
LLIYTTSSLKSGVPSRESGSGSGTDFTLTISRLQPEDFATYYCQQ
SYSSTWTFGRGTKVEIK
(SEQ ID NO: 363)
QSVLTQPPSVSAAPGQKVTISCSGNNSNIANNYVSWYQQLPGTAP
KLLIYDNNYRPSGIPDRESGSKSGTSATLDITGLQTGDEADYYCG
VWDGSLTTGVFGGGTKLTVL
(SEQ ID NO: 364)
LPVLTQPASVSGSPGQSITISCTGTTSDIGGYDYVSWYQQHPGKA
PKLMIYDVSKRPSGVSNRFSGSKSGNTASLTISGLQAEDEADYYC
SSYTSSSTHVFGTGTKLTVL
(SEQ ID NO: 365)
QSALTQPASVSGSPGQSITISCTGTSSDVGGYNYVSWYQQHPGKA
PKLMIYDVSNRPSGVSNRFSGSKSGNTASLTISGLQAEDEADYYC
SSYRSSTLGPVFGGGTKLTVL
(SEQ ID NO: 366)
QAGLTQPPSVSEAPRQRVTISCSGSSSNIGNNAVNWYQQLPGKAP
KLLIYYDDLLPSGVSDRESGSKSGTSASLAISGLQSEDEADYYCA
AWDDSLNGYVFGTGTKLTVL
(SEQ ID NO: 367)
QSALTQPRSVSGSPGQSVTISCTGTSSDVGGYNYVSWYQQHPGKA
PKLMIYDVSKRPSGVPDRFSGSKSGNTASLTISGLQAEDEADYYC
SSYTSSTTHVFGTGTKVTVL
(SEQ ID NO: 368)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSVWVEGGGTQLTVL
(SEQ ID NO: 369)
QSVLTQPASVSGSPGQSITISCTGTSSDVGGYNYVSWYQQHPGRA
PRLMIYDVSNRPSGVSNRFSGSKSGNTASLTISGLQAEDEGDYYC
SSYTSGGTLGPVFGGGTKLTVL
(SEQ ID NO: 370)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSAVVFGGGTKLTVL
(SEQ ID NO: 371)
QSVVTQPPSVSAAPGQKVTISCSGSSSNIGNNYVSWYQQVPGTAP
KLLIYDNNKRPSGIPDRESGSNSDTSATLGITGLQTGDEADYYCG
TWDSSLSAWVFGGGTKLTVL
(SEQ ID NO: 372)
QSVVTQPPSVSAAPGQKVTISCSGSSSNIGNNYVSWYQQLPGTAP
KLLIYDNNKRPSGIPDRESGSKSGTSATLGITGLQTGDEADYYCG
TWDSSLSAGSVVFGGGTKLTVL
(SEQ ID NO: 373)
SYELMQPPSVSVAPGKTATIACGGENIGRKTVHWYQQKPGQAPVL
VIYYDSDRPSGIPERFSGSNSGNTATLTISRVEAGDEADYYCLVW
DSSSDHRIFGGGTKLTVL
(SEQ ID NO: 374)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL
(SEQ ID NO: 375)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL
(SEQ ID NO: 376)
SYELMQPPSVSVAPGKTATIACGGENIGRKTVHWYQQKPGQAPVL
VIYYDSDRPSGIPERFSGSNSGNTATLTISRVEAGDEADYYCQVW
DSSSDHRIFGGGTKLTVL

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 377)
QVQLVQSGSEVKKSGSSVKVSCKTSGGTFSITNYAINWVRQAPGQ
GLEWMGGILPIFGAAKYAQKFQDRVTITADESTNTAYLELSSLTS
EDTAMYYCARGKRWLQSDLQYWGQGTLVTVSS
VL Sequences:
(SEQ ID NO: 378)
QPVLTQPASVSGSPGQSITISCTGSSSDVGSYDLVSWYQQSPGKV
PKLLIYEGVKRPSGVSNRFSGSKSGNTASLTISGLQAEDEADYYC
SSYAGTRNFVFGGGTQLTVL

In some embodiments, the PDL1 binding domain comprises or is derived from an antibody sequence or antigen-binding fragment thereof that includes a combination of a VH sequence and a VL sequence selected from the group consisting of:

VH Sequences:
(SEQ ID NO: 379)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKSSAGQSRPGFDYWGQGTLVTVSS
(SEQ ID NO: 380)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKSSAGQSWPGFDYWGQGTLVTVSS
(SEQ ID NO: 381)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKSSAGQSFPGFDYWGQGTLVTVSS_
(SEQ ID NO: 382)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKWSAAFDYWGQGTLVTVSS
(SEQ ID NO: 383)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKWSAGYDYWGQGTLVTVSS
(SEQ ID NO: 384)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKWSKGFDYWGQGTLVTVSS
(SEQ ID NO: 385)
EVQLLESGGGLVQPGGSLRLSCAASGFTFSSYAMSWVRQAPGKGL
EWVSSIWKQGIVTVYDSVKGRFTISRDNSKNTLYLQMNSLRAEDT
AVYYCAKSSAGFDYWGQGTLVTV
(SEQ ID NO: 386)
EVQLLESGGGLVQPGGSLRLSCAASGFTFSSYAMSWVRQAPGKGL
EWVSSIWRNGIVTVYDSVKGRFTISRDNSKNTLYLQMNSLRAEDT
AVYYCAKSSAGFDYWGQGTLVTVSS
(SEQ ID NO: 387)
EVQLLESGGGLVQPGGSLRLSCAASGFTFSSYAMSWVRQAPGKGL
EWVSDIWKQGMVTVYDSVKGRFTISRDNSKNTLYLQMNSLRAEDT
AVYYCAKSSAGFDYWGQGTLVTVSS
(SEQ ID NO: 388)
EVQLLESGGGLVQPGGSLRLSCAASGFTFSSYAMSWVRQAPGKGL
EWVSSIWRQGLATAYDSVKGRFTISRDNSKNTLYLQMNSLRAEDT
AVYYCAKSSAGFDYWGQGTLVTVSS
(SEQ ID NO: 389)
EVQLLESGGGLVQPGGSLRLSCAASGFTFSSYAMSWVRQAPGKGL
EWVSEIVATGILTSYDSVKGRFTISRDNSKNTLYLQMNSLRAEDT
AVYYCAKSSAGFDYWGQGTLVTVSS
(SEQ ID NO: 390)
EVQLLESGGGLVQPGGSLRLSCAASGFTFSSYAMSWVRQAPGKGL
EWVSSIGRQGLITVYDSVKGRFTISRDNSKNTLYLQMNSLRAEDT
AVYYCAKSSAGFDYWGQGTLVTVSS
(SEQ ID NO: 391)
EVQLLESGGGLVQPGGSLRLSCAASGFTFSSYAMSWVRQAPGKGL
EWVSSIWYQGLVTVYDSVKGRFTISRDNSKNTLYLQMNSLRAEDT
AVYYCAKSSAGFDYWGQGTLVTVSS
(SEQ ID NO: 392)
EVQLLESGGGLVQPGGSLRLSCAASGFTFSSYAMSWVRQAPGKGL
EWVSDIWKQGFATADSVKGRFTISRDNSKNTLYLQMNSLRAEDTA
VYYCAKSSAGFDYWGQGTLVTVSS
(SEQ ID NO: 393)
EVQLLESGGGLVQPGGSLRLSCAASGFTFSSYAMSWVRQAPGKGL
EWVSSIWKQGIVTVYDSVKGRFTISRDNSKNTLYLQMNSLRAEDT
AVYYCAKSSAGFDYWGQGTLVTVSS
(SEQ ID NO: 394)
EVQLLESGGGLVQPGGSLRLSCAASGFTFSSYAMSWVRQAPGKGL
EWVSSIWRQGLATAYDSVKGRFTISRDNSKNTLYLQMNSLRAEDT
AVYYCAKSSAGFDYWGQGTLVTVSS
(SEQ ID NO: 395)
EVQLLESGGGLVQPGGSLRLSCAASGFTFSSYAMSWVRQAPGKGL
EWVSSIWRNGIVTVYADSVKGRFTISRDNSKNTLYLQMNSLRAED
TAVYYCAKWSAAFDYWGQGTLVTVSS
(SEQ ID NO: 396)
EVQLLESGGGLVQPGGSLRLSCAASGFTFSSYAMSWVRQAPGKGL
EWVSSIWRNGIVTVYADSVKGRFTISRDNSKNTLYLQMNSLRAED
TAVYYCAKWSAGYDYWGQGTLVTVSS
(SEQ ID NO: 397)
EVQLLESGGGLVQPGGSLRLSCAASGFTFSSYAMSWVRQAPGKGL
EWVSSIWRNGIVTVYADSVKGRFTISRDNSKNTLYLQMNSLRAED
TAVYYCAKWSKGFDYWGQGTLVTVSS
(SEQ ID NO: 398)
EVQLLESGGGLVQPGGSLRLSCAASGFTFSSYAMetSWVRQAPGK
GLEWVSSIWYQGLVTVYADSVKGRFTISRDNSKNTLYLQMetNSL
RAEDTAVYYCAKWSAAFDYWGQGTLVTVSS
(SEQ ID NO: 399)
EVQLLESGGGLVQPGGSLRLSCAASGFTFSSYAMSWVRQAPGKGL
EWVSSIWYQGLVTVYADSVKGRFTISRDNSKNTLYLQMNSLRAED
TAVYYCAKWSAGYDYWGQGTLVTVSS
(SEQ ID NO: 400)
EVQLLESGGGLVQPGGSLRLSCAASGFTFSSYAMSWVRQAPGKGL
EWVSSIWYQGLVTVYADSVKGRFTISRDNSKNTLYLQMNSLRAED
TAVYYCAKWSKGFDYWGQGTLVTVSS
VL Sequences:
(SEQ ID NO: 401)
DIQMTQSPSSLSASVGDRVTITCRASQSISSYLNWYQQKPGKAPK
LLIYYASTLQSGVPSRESGSGSGTDFTLTISSLQPEDFATYYCQQ
DNGYPSTFGQGTKVEIKR
(SEQ ID NO: 402)
DIQMTQSPSSLSASVGDRVTITCRASQSISSYLNWYQQKPGKAPK
LLIYYASTLQSGVPSRESGSGSGTDFTLTISSLQPEDFATYYCQQ
DNGYPSTFGQGTKVEIKR
(SEQ ID NO: 403)
DIQMTQSPSSLSASVGDRVTITCRASQSISSYLNWYQQKPGKAPK
LLIYAASSLQSGVPSRESGSGSGTDFTLTISSLQPEDFATYYCQQ
DNGYPSTFGGGTKVEIKR

In some embodiments, the PDL1 binding domain comprises or is derived from an antibody sequence or antigen-binding fragment thereof that includes a single chain Fv (scFv) sequence selected from the group consisting of:

(SEQ ID NO: 404)
EVQLLESGGGLVQPGGSLRLSCAASGFTFSSYAMSWVRQAPGKGL
EWVSDITASGQRTTYADSVKGRFTISRDNSKNTLYLQMNSLRAED
TAVYYCARSKIAFDYWGQGTLVTVSSGGGGSGGGGSGGGGSTDIQ
MTQSPSSLSASVGDRVTITCRASQSISSYLNWYQQKPGKAPKLLI
YKASRLQSGVPSRFSGSGSGTDFTLTISSLQPEDFATYYCQQRAL
KPVTFGQGTKVEIKR
(SEQ ID NO: 405)
EVQLLESGGGLVQPGGSLRLSCAASGFTFSSYAMSWVRQAPGKGL
EWVSSINKDGHYTSYADSVKGRFTISRDNSKNTLYLQMNSLRAED
TAVYYCAKNLDEFDYWGQGTLVTVSSGGGGSGGGGSGGGGSTDIQ
MTQSPSSLSASVGDRVTITCRASQSISSYLNWYQQKPGKAPKLLI
YAASSLQSGVPSRFSGSGSGTDFTLTISSLQPEDFATYYCQQSYS
TPNTFGQGTKVEIKR
(SEQ ID NO: 406)
EVQLLESGGGLVQPGGSLRLSCAASGFTFSSYAMSWVRQAPGKGL
EWVSSIMATGAGTLYADSVKGRFTISRDNSKNTLYLQMNSLRAED
TAVYYCAKDGAGFDYWGQGTLVTVSSGGGGSGGGGSGGGGSTDIQ
MTQSPSSLSASVGDRVTITCRASQSISSYLNWYQQKPGKAPKLLI
YSASQLQSGVPSRFSGSGSGTDFTLTISSLQPEDFATYYCQQANS
RPSTFGQGTKVEIKR
(SEQ ID NO: 407)
EVQLLESGGGLVQPGGSLRLSCAASGFTFSSYAMSWVRQAPGKGL
QWVSTITSSGAATYYADSVKGRFTISRDNSKNTLYLQMNSLRAED
TAVYYCAKNYTGFDYWGQGTLVTVSSGGGGSGGGGSGGGGSTDIQ
MTQSPSSLSASVGDRVTITCRASQSISSYLNWYQQKPGKAPKLLI
YNASSLQSGVPSRFSGSGSGTDFTLTISSLQPEDFATYYCQQYTY
GPGTFGQGTKVEIKR
(SEQ ID NO: 408)
EVQLLESGGGLVQPGGSLRLSCAASGFTFSSYAMSWVRQAPGKGL
EWVSSIYSTGGATAYADSVKGRFTISRDNSKNTLYLQMNSLRAED
TAVYYCAKSSAGFDYWGQGTLVTVSSGGGGSGGGGSGGGGSTDIQ
MTQSPSSLSASVGDRVTITCRASQSISSYLNWYQQKPGKAPKLLI
YYASTLQSGVPSRFSGSGSGTDFTLTISSLQPEDFATYYCQQDNG
YPSTFGQGTKVEIKR

### PDL1×41BB Dual Targeting

In some embodiments, the fusion proteins are bispecific molecules that include a TBD that binds 41BB and a binding domain directed toward PDL1. In these, embodiments, the binding to PDL1 is capable of providing the additional crosslinking function and TNFRSF activation can be achieved with only one or two anti-41BB TBDs. In these embodiments, the TNFRSF signaling is enhanced and focused by the presence of a PDL1 expressing cell.

Tetravalent 41BB agonist: hzRH3v5-1
(SEQ ID NO: 448)
EVQLLESGGGEVQPGGSLRLSCAASGFSFSINAMGWYRQAPGKRREFVAAIESGRNTVYAESVK
GRFTISRDNAKNTVYLQMSSLRAEDTAVYYCGLLKGNRVVSPSVAYWGQGTLVTVKPGGGGDKT
HTCPPCPAPGGPSVFLFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQ
QGNVFSCSVMHEALHNHYTQKSLSLSPGSGGGGSGGGGSEVQLLESGGGEVQPGGSLRLSCAAS
GFSFSINAMGWYRQAPGKRREFVAAIESGRNTVYAESVKGRFTISRDNAKNTVYLQMSSLRAED
TAVYYCGLLKGNRVVSPSVAYWGQGTLVTVKPGG
Bispecific PDL1 x 41BB: hz28A2v5 x hzRH3v5-1
(SEQ ID NO: 449)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQPGGSLRLSCAASGFSFSINAMGWYRQAPGKRREFVAAIESGRNTVYAESVKGRFTISRDNAK
NTVYLQMSSLRAEDTAVYYCGLLKGNRVVSPSVAYWGQGTLVTVKPGGGGDKTHTCPPCPAPGG
PSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMH
EALHNHYTQKSLSLSPGK
Bispecific PDL1 x 41BB: hz28A2v5 x hzRH3v5-2
(SEQ ID NO: 450)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQPGGSLRLSCAASGFSFSINAMGWYRQAPGKRREFVAAIYSGRNTVYAESVKGRFTISRDNAK
NTVYLQMSSLRAEDTAVYYCGLLKGNRVVSPSVAYWGQGTLVTVKPGGGGDKTHTCPPCPAPGG
PSVFLFPPKPKDTLMISRTPEVTCVVVDVSHEDPEVKENWYVDGVEVHNAKTKPREEQYNSTYR
VVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMH
EALHNHYTQKSLSLSPGK
Bispecific PDL1 x 41BB: hz28A2v5 x hzRH3v5-16
(SEQ ID NO: 451)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQPGGSLRLSCAASGFSFSINAMGWYRQAPGKRREFVAAIYSGSSTVYAESVKGRFTISRDNAK
NTVYLQMSSLRAEDTAVYYCGLLKGNRVVSPSVAYWGQGTLVTVKPGGGGDKTHTCPPCPAPGG
PSVFLFPPKPKDTLMISRTPEVTCVVVDVSHEDPEVKENWYVDGVEVHNAKTKPREEQYNSTYR
VVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMH
EALHNHYTQKSLSLSPGK
Bispecific PDL1 x 41BB: hz28A2v5 x hz4E01v16
(SEQ ID NO: 452)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQLLESGGGEVQPGGSLRLSCAASGWAFGNYGMAWFRQAPGKEREFVSRLAWQGGSTDYVESVK
GRFTISRDNAKNTLYLQMSSLRAEDTAVYYCARQRSYSRYDIRTPQTYDYWGQGTLVTVKPGGG
GDKTHTCPPCPAPGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Bispecific PDL1 x 41BB: hz28A2v5 x hz4E01v18
(SEQ ID NO: 453)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQLLESGGGEVQPGGSLRLSCAASGWAFGNYGMAWFRQAPGKEREFVSRLAWGGGSTDYVESVK
GRFTISRDNAKNTLYLQMSSLRAEDTAVYYCARQRSYSRYDIRTPQTYDYWGQGTLVTVKPGGG
GDKTHTCPPCPAPGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Bispecific PDL1 x 41BB: hz28A2v5 x hz4E01v21
(SEQ ID NO: 454)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQLLESGGGEVQPGGSLRLSCAASGWAFSNYGMAWFRQAPGKEREFVSRLAWGGGSTDYVESVK
GRFTISRDNAKNTLYLQMSSLRAEDTAVYYCARQRSYSRYDIRTPQTYDYWGQGTLVTVKPGGG
GDKTHTCPPCPAPGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Bispecific PDL1 x 41BB: hz28A2v5 x hz4E01v22
(SEQ ID NO: 455)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQLLESGGGEVQPGGSLRLSCAASGWAFGNYGMAWFRQAPGKEREFVSRLAWSGGSTDYVESVK
GRFTISRDNAKNTLYLQMSSLRAEDTAVYYCARQRSYSRYDIRTPQTYDYWGQGTLVTVKPGGG
GDKTHTCPPCPAPGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Bispecific PDL1 x 41BB: hz28A2v5 x hz4E01v23
(SEQ ID NO: 456)
EVQLLESGGGEVQPGGSLRLSCAASGGIFAIKPISWYRQAPGKQREWVSTTTSSGATNYAESVK
GRFTISRDNAKNTLYLQMSSLRAEDTAVYYCNVFEYWGQGTLVTVKPGGSGGSEVQLLESGGGE
VQLLESGGGEVQPGGSLRLSCAASGWAFSNYGMAWFRQAPGKEREFVSRLAWSGGSTDYVESVK
GRFTISRDNAKNTLYLQMSSLRAEDTAVYYCARQRSYSRYDIRTPQTYDYWGQGTLVTVKPGGG
GDKTHTCPPCPAPGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK

In some embodiments, the fusion proteins are multispecific containing a TBD and a binding domain directed toward Folate Receptor Alpha (FRα). In these, embodiments, the binding to FRα is capable of providing the additional crosslinking function and TNFRSF activation can be achieved with only one or two TBDs. In these embodiments, the TNFRSF signaling is enhanced and focused by the presence of a FRα expressing cell.

Exemplary FRα-targeting single domain sequences are shown below:

Fra-5:
(SEQ ID NO: 409)

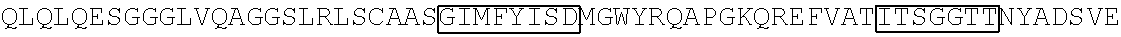

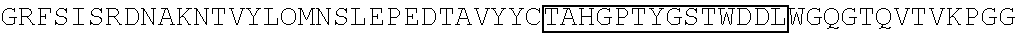

(SEQ ID NO: 410)
CDR1: GIMFYISD
(SEQ ID NO: 411)
CDR2: TITSGGTTNY
(SEQ ID NO: 412)
CDR3: TAHGPTYGSTWDDL
Fra-6:
(SEQ ID NO: 413)

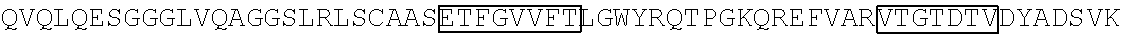

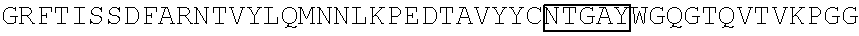

(SEQ ID NO: 414)
CDR1: TFGVVFT
(SEQ ID NO: 415)
CDR2: VIGTDTV
(SEQ ID NO: 416)
CDR3: NTGAY
Fra-57:
(SEQ ID NO: 417)

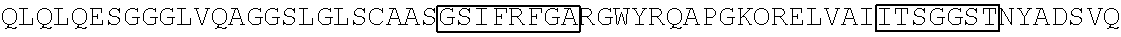

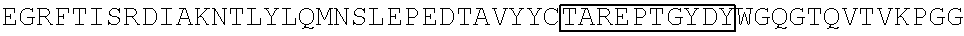

(SEQ ID NO: 418)
CDR1: GRTASTYS
(SEQ ID NO: 419)
CDR2: IWSTGST
(SEQ ID NO: 420)
CDR3: TAREPTGYDY
1A3:
(SEQ ID NO: 410)

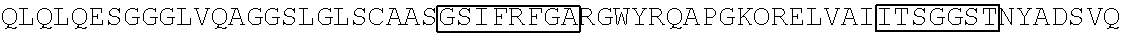

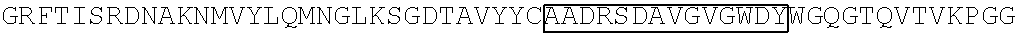

(SEQ ID NO: 422)
CDR1: GSIFREGA
(SEQ ID NO: 423)
CDR2: ITSGGST
(SEQ ID NO: 424)
CDR3: AADRSDAVGVGWDY
1F3:
(SEQ ID NO: 425)

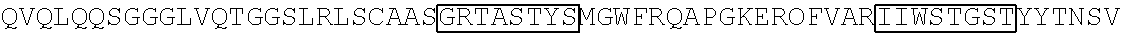

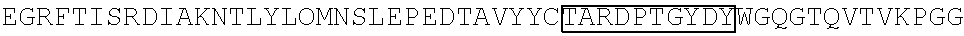

(SEQ ID NO: 418)
CDR1: GRTASTYS
(SEQ ID NO: 426)
CDR2: IIWSTGST
(SEQ ID NO: 427)
CDR3: TARDPTGYDY
1G10:
(SEQ ID NO: 428)

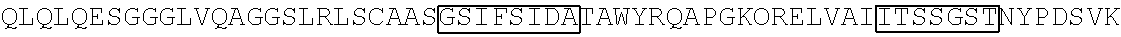

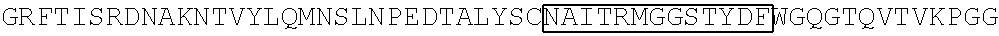

(SEQ ID NO: 429)
CDR1: GSIFSIDA
(SEQ ID NO: 430)
CDR2: ITSSGST
(SEQ ID NO: 431)
CDR3: NAITRMGGSTYDF

The disclosure will be further described in the following examples, which do not limit the scope of the disclosure described in the claims.

### EXAMPLES

### Example 1. 41BB-Targeting Single Domain Antibodies Bind 41BB

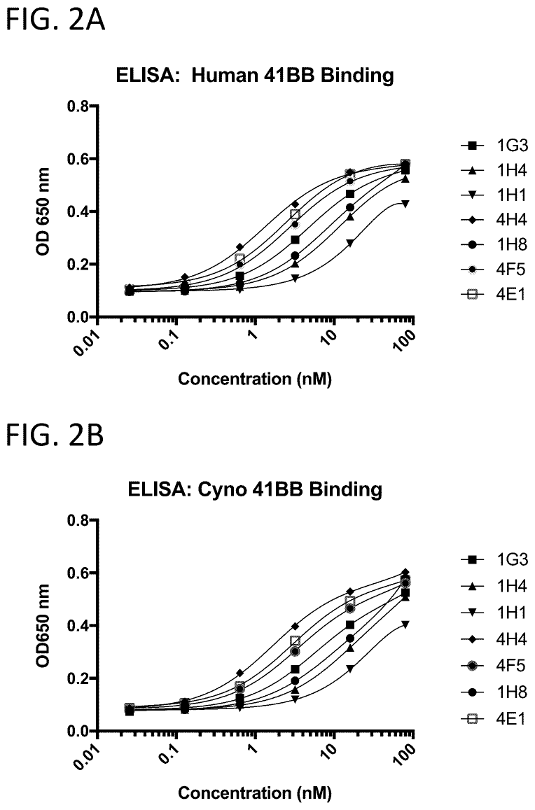

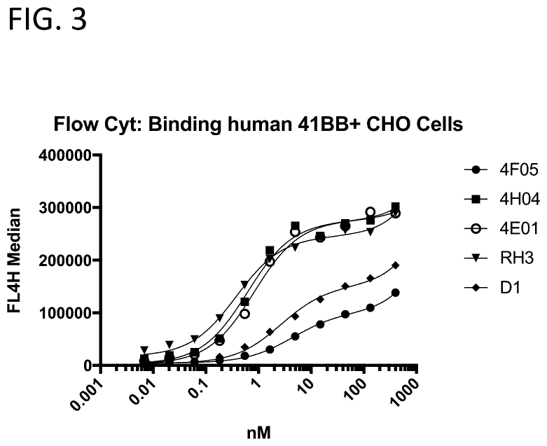

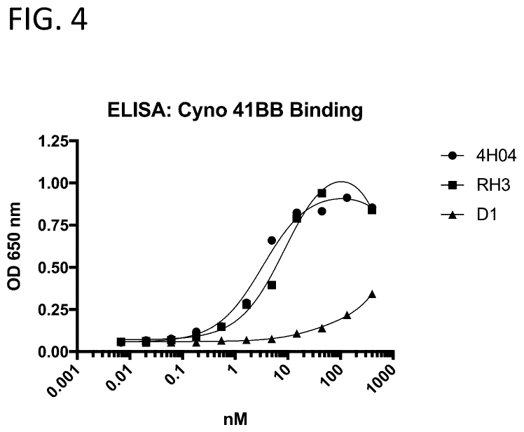

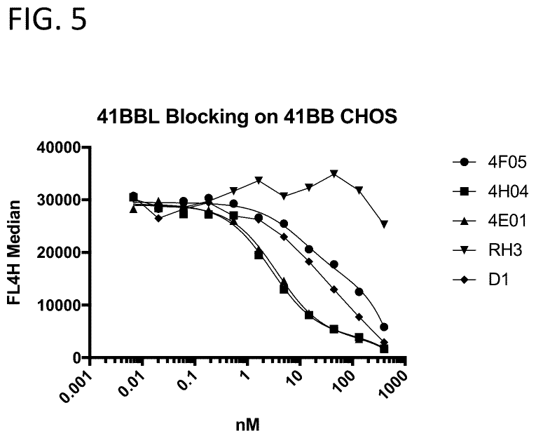

The 41BB-targeting single domain antibodies (sdAbs) referred to herein as 1G3 (SEQ ID NO: 432), 1H4 (SEQ ID NO: 436), 1H1 (SEQ ID NO: 440), 4H4 (SEQ ID NO: 16), 1H8 (SEQ ID NO: 444), 4F5 (SEQ ID NO: 23), and 4E1 (SEQ ID NO: 20) bind recombinant human 41BB (FIG. 2A), cynomolgus 41BB (FIG. 2B). The 41BB-targeting single domain antibodies (sdAbs) referred to herein as 4F5 (SEQ ID NO: 23), 4H04 (SEQ ID NO: 16), 4E01 (SEQ ID NO: 20), RH03 (SEQ ID NO: 25), and D1 (SEQ ID NO: 29) bind human 41BB expressed on the cell surface of CHO cells (FIG. 3). The 41BB-targeting sdAbs referred to herein as 4H04, RH03, and bind cynomolgus 41BB. For FIG. 2A, FIG. 2B, and FIG. 4, binding was assessed by ELISA wherein recombinant 41BB-mFc fusion protein (a fusion protein containing 41BB operably linked to a mouse Fc region) was immobilized on a Medisorp 96 well plate. For FIG. 3, binding was assessed by flow cytometry using 41BB expressing CHO cells, and the data is presented as median fluorescence intensity.

### Example 2. 41BB-Targeting Single Domain Antibodies Block 41BB

The 41BB-targeting single domain antibodies (sdAbs) referred to herein as 4F05 (SEQ ID NO: 23), 4H04 (SEQ ID NO: 16), 4E01 (SEQ ID NO: 20), RH03 (SEQ ID NO: 25), and D1 (SEQ ID NO: 29) block the interaction between 41BB and its ligand 41BBL. All single domain antibodies tested, with the exception of RH3 blocks the interaction between 41BB and 41BBL. Blocking was assessed by flow cytometry using a recombinant 41BB fusion protein and 41BB expressing CHO cells, data is presented as median fluorescence intensity.

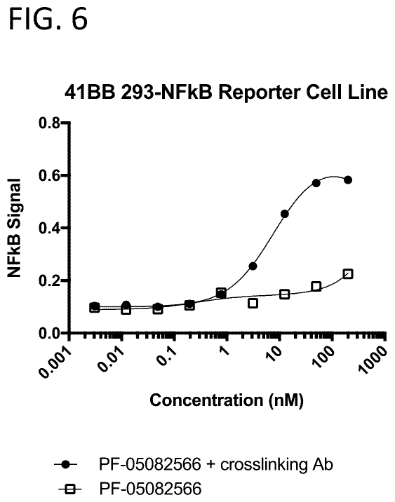

In contrast to the 41BB sdAbs of the disclosure, conventional bivalent anti-41BB antibodies do not induce 41BB signaling unless further clustered with an exogenous crosslinking anti-human IgG antibody. FIG. 6 demonstrates the inability of a conventional bivalent anti-41BB antibody PF-05082566, which is disclosed in U.S. Pat. No. 8,337,850, to induce 41BB signaling unless further clustered with an exogenous crosslinking anti-human IgG antibody. In FIG. 6, 41BB signaling was monitored using a NF-kB reporter 293 cell line expressing 41BB.

### Example 3. PDL1-Targeting Single Domain Antibodies Bind PDL1 and Block the Interaction Between PLD1 and PD1

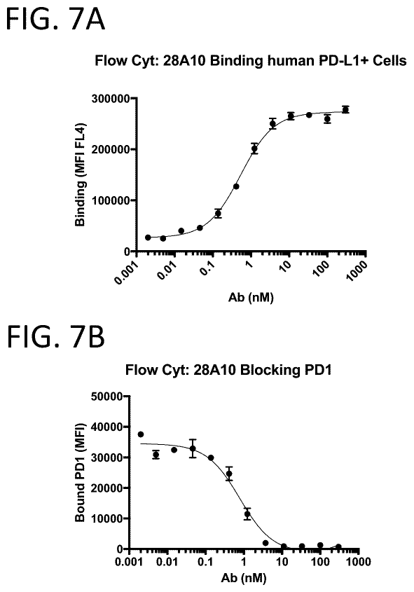

The studies presented herein use an exemplary PDL1 single domain antibody (sdAb), referred to herein as 28A10 (SEQ ID NO: 100) to demonstrate that the PDL1-targeting sdAbs of the disclosure bind cell surface PDL1 (FIG. 7A) and block the interaction of PDL1 with PD1 (FIG. 7B). Binding was assessed by flow cytometry on PDL1 expressing CHO cells, and blocking was assessed by flow cytometry using a recombinant PD1 fusion protein and PDL1 expressing CHO cells. The data presented in FIGS. 7A and 7B are presented as median fluorescence intensity.

### Example 4. PDL1-41BB Targeting Fusion Proteins

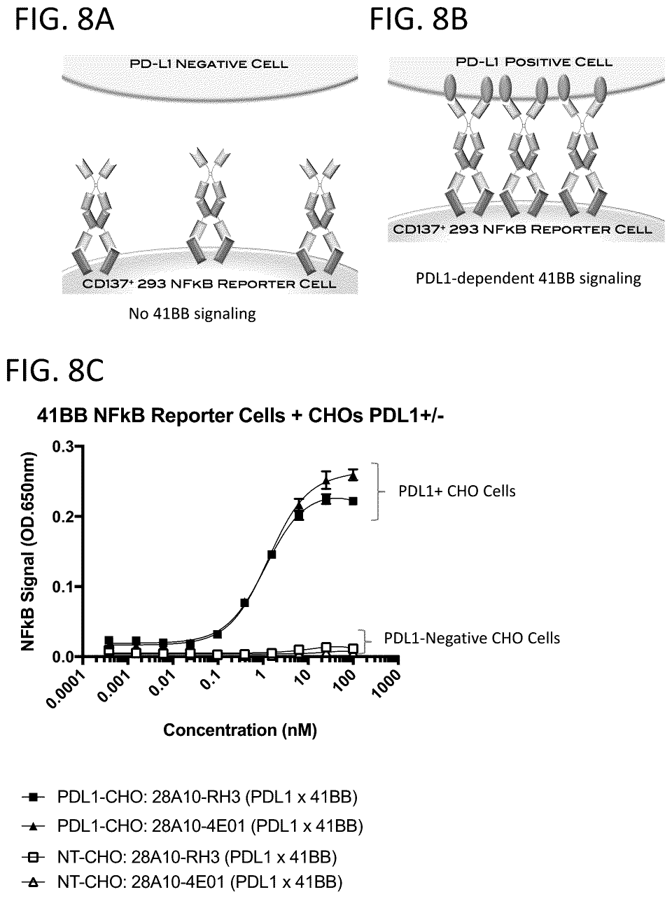

The disclosure provides fusion proteins that target at least PDL1 and 41BB. These bispecific PDL1-41BB targeting fusion proteins are agonists of PDL1-dependent 41BB mediated signaling. FIGS. 8A and 8B are conceptual schematics wherein the bispecific fusion proteins have minimal 41BB agonistic properties (FIG. 8A) unless bound by a PD-L1 expressing cell (FIG. 8B). FIG. 8C demonstrates the ability of a PDL1-positive cell, in this case, a population of PDL1 transfected CHO cells, to mediate 41BB signaling and the inability of PDL1-negative cell, in this case, a population of untransfected CHO cells, to mediate 41BB signaling. Two distinct bispecific fusion proteins are shown in this figure, each containing a distinct 41BB binding VHH (e.g., 4EOlor RH3) and the same PD-L1 VHH, 28A10. 41BB signaling was monitored using a NF-kB reporter 293 cell line expressing 41BB. This reporter cell line implements an NF-kB driven secreted alkaline phosphatase, to monitor NF-kB signaling.

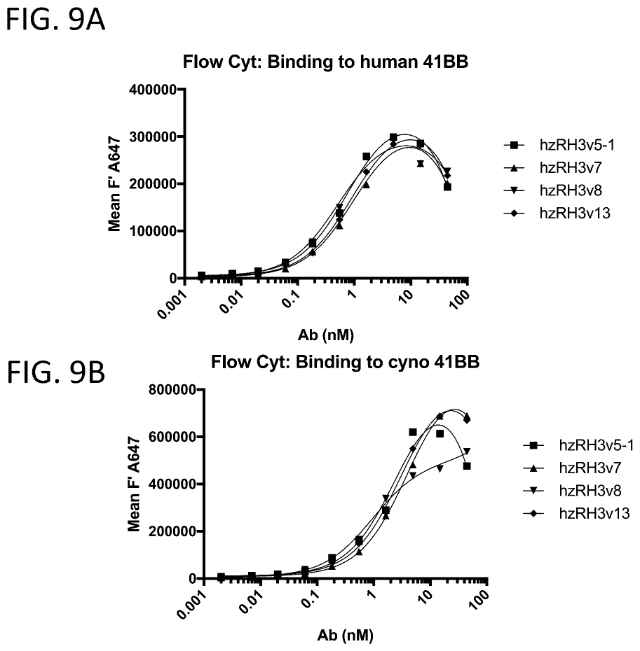

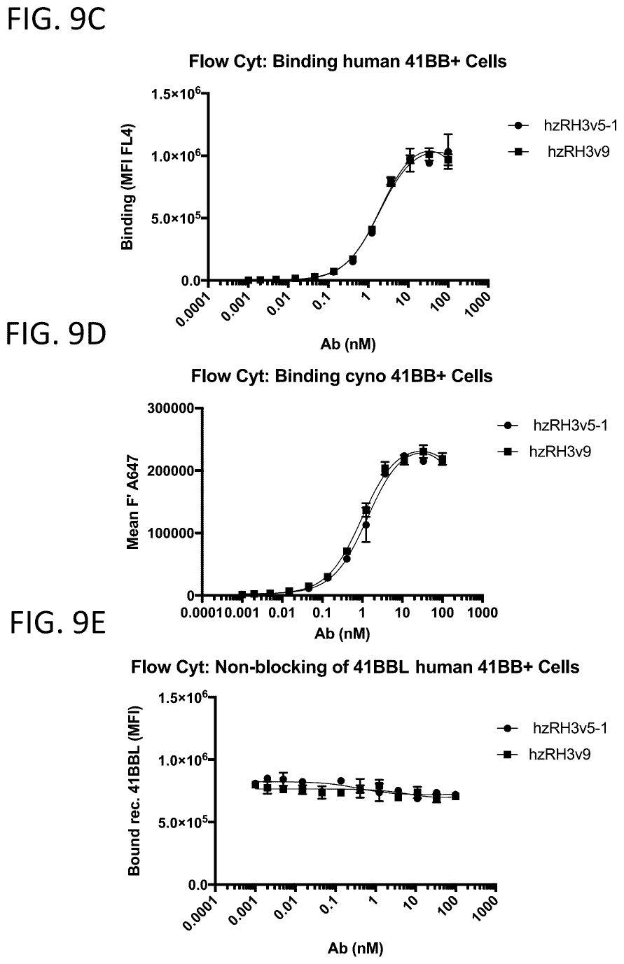

The PDL1-41BB targeting fusion proteins of the disclosure include a humanized anti-41BB sequence. In the studies presented herein, the PDL1-41BB targeting fusion proteins of the disclosure include a humanized anti-41BB sequence such as hzRH3v5-1 (SEQ ID NO: 30) and/or hzRH3v9 (SEQ ID NO: 82) bind both human and cynomolgus 41BB (FIGS. 9A, 9B), including human 41BB and cynomolgus 41BB expressed on the surface of CHO cells (FIGS. 9C, 9D). Binding was assessed by flow cytometry on 41BB expressing 293freestyle cells.

The humanized variants hzRH3v5-1 and hzRH3v9 do not block binding of 41BBL to cell surface 41BB as shown in FIG. 9E. In these studies, a recombinant fusion protein 41BBL-mFc, containing a mouse Fc region, was used, and bound 41BBL was detected using an anti-mouse IgG-Fc specific secondary antibody.

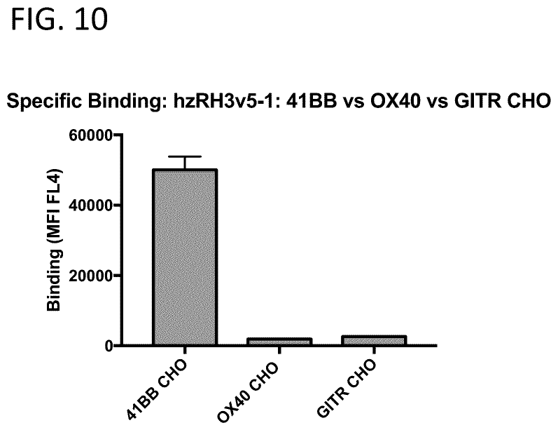

The humanized variant hzRH3v5-1 specifically binds 41BB as compared to the other TNFRSF members OX40 and GITR (FIG. 10). Binding was assessed by flow cytometry using CHO cells expressing the given TNFRSF member.

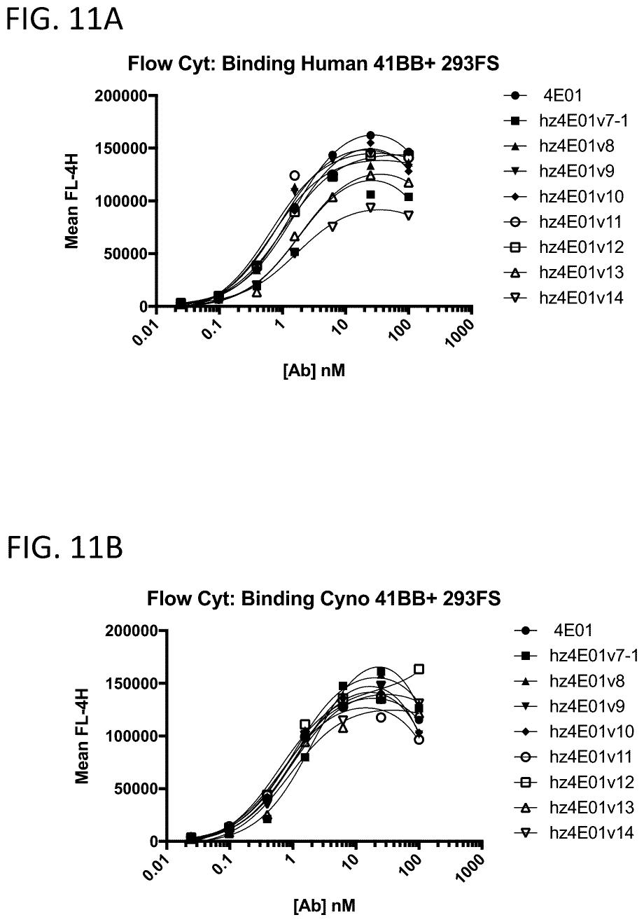

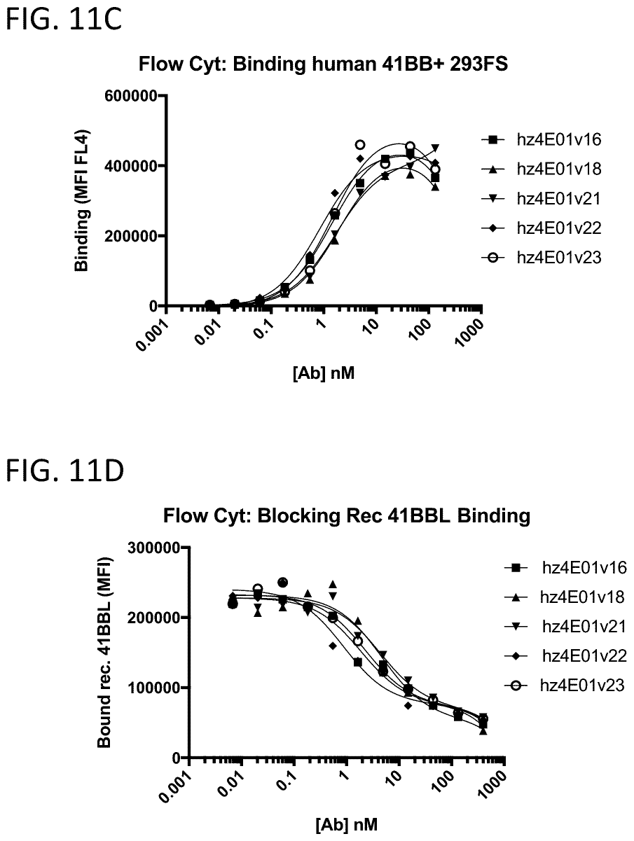

Additional humanized 41BB variants were analyzed. FIGS. 11A, 11B, 11C, and 11D demonstrate the binding to human (FIG. 11A and FIG. 11C) or cynomolgus monkey (FIG. 11B) 41BB of the humanized 4E01 variants. Binding was assessed by flow cytometry on 41BB expressing 293freestyle cells. FIG. 11D demonstrates that the humanized variants hz4E01v16, hz4E01v18, hz4E01v21, hz4E01v22 and hz4E01v23 block binding of 41BBL to cell surface 41BB. In these studies, a recombinant fusion protein 41BBL-mFc, containing a mouse Fc region was used and bound 41BBL was detected using an anti-mouse IgG-Fc specific secondary antibody.

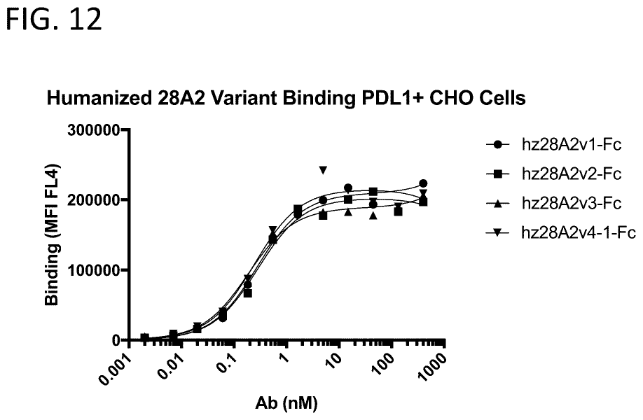

The PDL1-41BB targeting fusion proteins of the disclosure also include a humanized anti-PDL1 sequence. In the studies presented herein, the PDL1-41BB targeting fusion proteins of the disclosure include a humanized anti-PDL1 sequence such as hz28A2v1 (SEQ ID NO: 120), hz28A2v2 (SEQ ID NO: 121), hz28A2v3 (SEQ ID NO: 122), and hz28A2v4-1 (SEQ ID NO: 123). FIG. 12 demonstrates binding of humanized single domain antibodies targeting PDL1. Binding was assessed by flow cytometry on PDL1-expressing CHO cells.

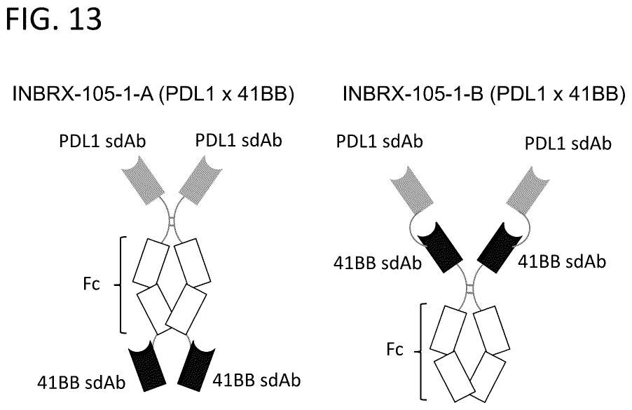

FIG. 13 is a schematic of two exemplary formats of a PDL1×41BB bispecific fusion protein of the disclosure, referred to herein as INBRX-105-1. INBRX-105-1-A (left) has the PDL1 and 41BB binding domains located at opposing terminal positions with a central Fc region, whereas INBRX-105-1-B (right) has the PDL1 and 41BB binding domains positioned in tandem, N-terminal to an Fc region.

These two formats were further evaluated for their ability to bind human or cynomolgus monkey 41BB, to block the interaction between 41BB and 41BBL, to bind PDL1, and to block the interaction between PDL1 and PD1.

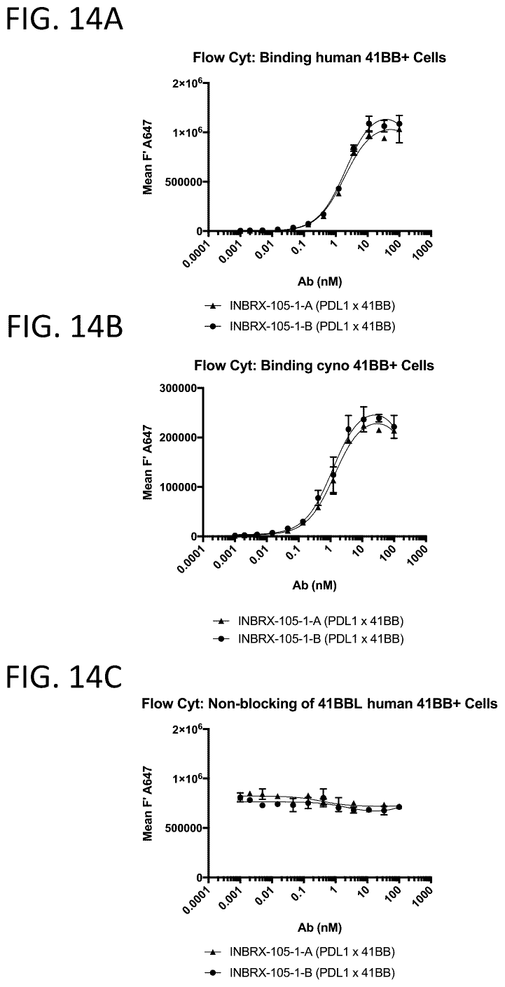

In particular, FIGS. 14A, 14B, and 14C demonstrate the equivalent binding to human (FIG. 14A) or cynomolgus monkey (FIG. 14B) 41BB by the two distinct formats of a bispecific fusion protein targeting PDL1 and 41BB referred to herein as INBRX-105-1-A and INBRX-105-1-B and illustrated in FIG. 13. Binding was assessed by flow cytometry on 41BB expressing 293freestyle cells. In the studies presented herein, hzRH3v5-1 (SEQ ID NO: 124) is the 41BB binding domain used in both formats. As shown in FIG. 14C, the bispecific fusion protein containing hzRh3v5-1 does not block 41BBL binding to cell surface 41BB. In these studies, a recombinant fusion protein of 41BBL and a mouse Fc region was used, and bound 41BBL was detected using an anti-mouse IgG-Fc specific secondary antibody.

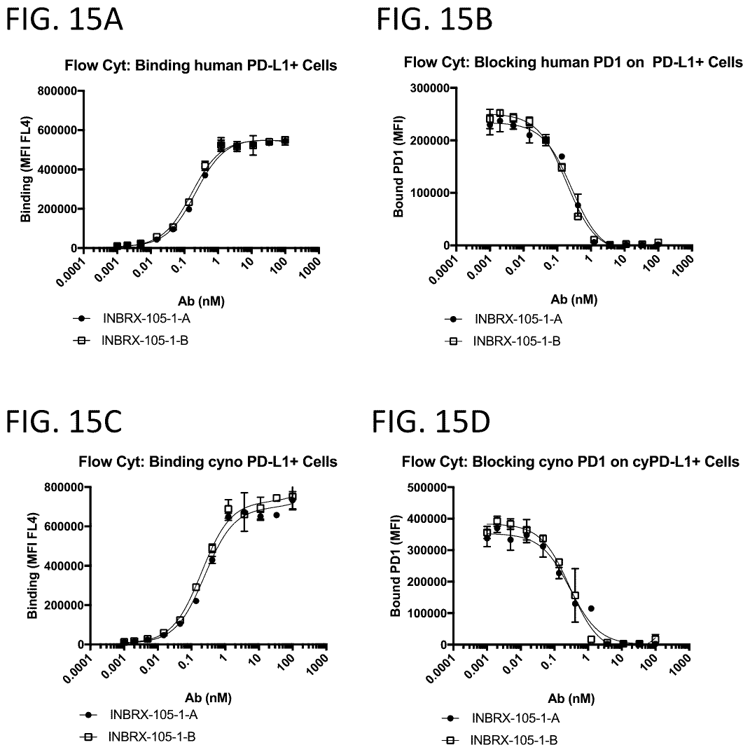

Furthermore, FIGS. 15A, 15B, 15C, and 15D demonstrate the equivalent binding (FIG. 15A and FIG. 15C) and PD1 blocking (FIG. 15B and FIG. 15D) by the two distinct formats of a bispecific fusion protein targeting PDL1 and 41BB referred to herein as INBRX-105-1-A and INBRX-105-1-B. Binding was assessed by flow cytometry on human (FIG. 15A) or cynomolgus monkey (FIG. 15C) PDL1 expressing 293freestyle cells. Blocking was assessed by flow cytometry using on human (FIG. 15B) or cynomolgus monkey (FIG. 15D) PDL1 expressing 293freestyle cells with either recombinant human (FIG. 15B) or cynomolgus monkey (FIG. 15D) PD1-mFc fusion protein. Bound PD1 was detected using an anti-mouse IgG-Fc specific secondary antibody. In the studies presented herein, hz28A2v5 is the PDL1-binding domain used in both formats.

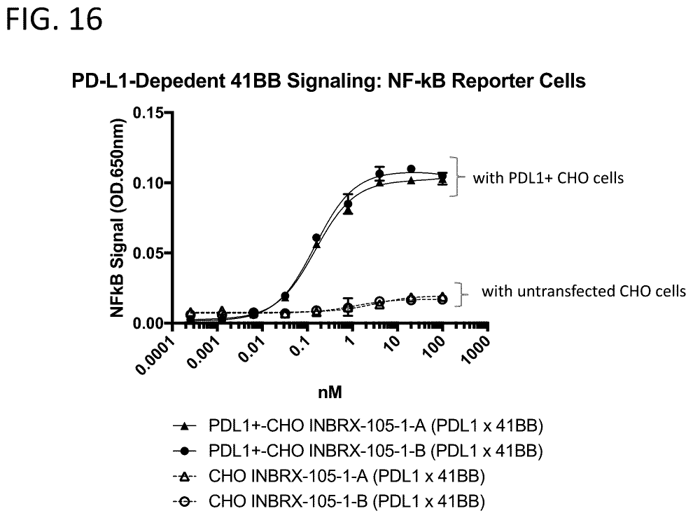

The PDL1×41BB bispecific fusion proteins were evaluated for their ability to induce PDL1-dependent 41BB agonism. FIG. 16 demonstrates the ability of humanized versions of a PDL1×41BB bispecific fusion protein (INBRX-105-1) to induce PDL1-dependent 41BB agonism. Compared herein are two distinct formats, INBRX-105-1-A vs INBRX-105-1-B, having the PDL1 and 41BB binding domains positioned at opposite termini or in tandem within the fusion protein, respectively. Notably, INBRX-105-1-A vs INBRX-105-1-B demonstrate equivalent PDL1-dependent agonistic activities. A 41BB-expressing HEK293 NF-kB reporter cell line was used to assess 41BB signaling and a PDL1-expressing CHO cell line was used as the source of PDL1. This reporter cell line implements an NF-kB driven secreted alkaline phosphatase, to monitor NF-kB signaling.

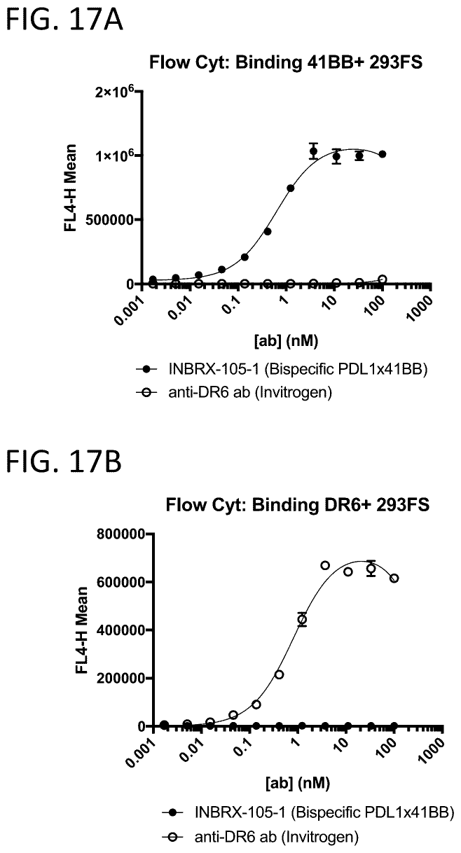

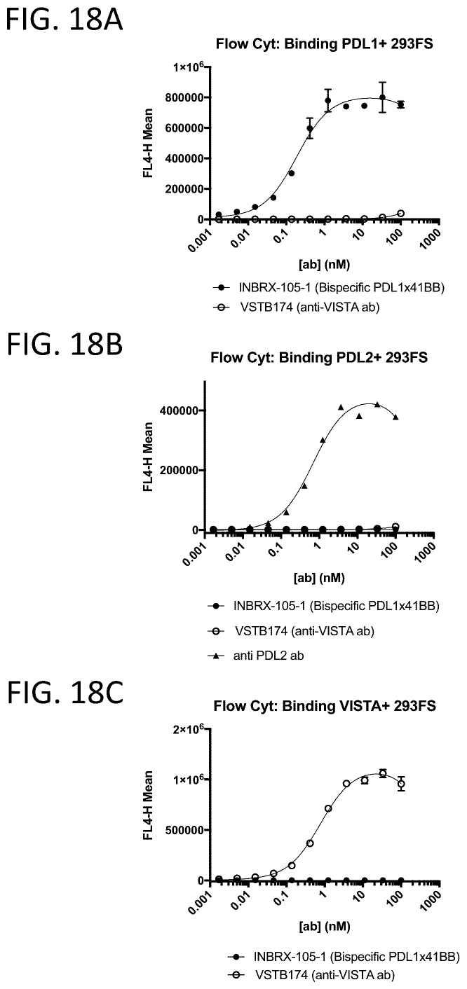

The ability of the 41BB-specific binding and the PDL1-specific binding by the binding domains in the PDL1×41BB bispecific fusion proteins was evaluated. FIGS. 17A and 17B demonstrate the 41BB-specific binding by the 41BB-binding portion of a PDL1×41BB bispecific fusion protein (INBRX-105-1) of the present disclosure. Binding was assessed on 41BB (FIG. 17A) or the closest homolog, TNFRSF21/DR6 (FIG. 17B), expressing 293freestyle cells by flow cytometry. An anti-DR6 antibody (Invitrogen) was used to as positive control for DR6 expression. In addition, FIGS. 18A, 18B, and 18C demonstrate the PDL1-specific binding by the PDL1-binding portion of a PDL1×41BB bispecific fusion protein (INBRX-105-1) of the present disclosure. Binding was assessed on PDL1 (FIG. 18A), the closest homologs PDL2 (FIG. 18B) or VISTA/PDL3 (FIG. 18C), expressing 293freestyle cells by flow cytometry. An anti-PDL2 antibody and an anti-VISTA antibody known as VSTB174, which is disclosed in PCT Publication No. WO 2015/097536, were used to as positive controls for PDL2 and PDL3 expression respectively.

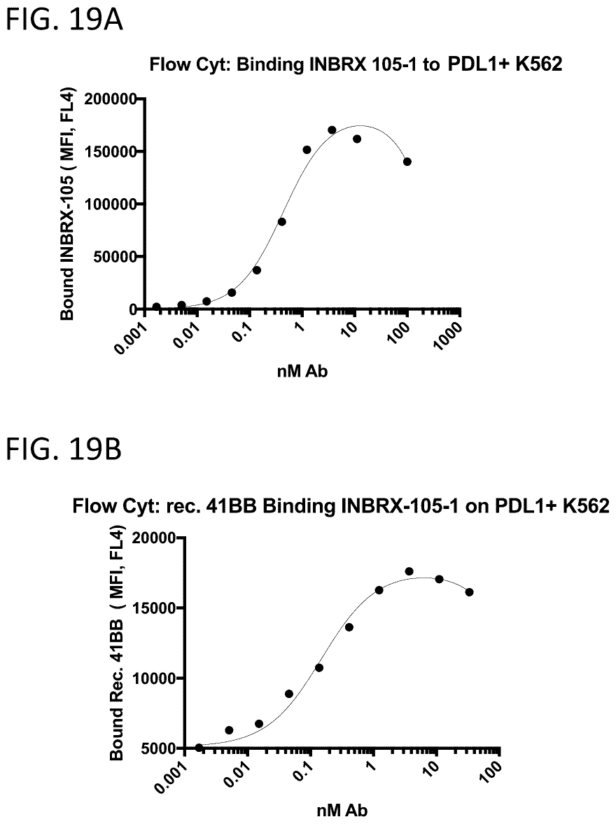

The ability of the PDL1×41BB bispecific fusion proteins to simultaneously bind both 41BB and PDL1 was evaluated. FIGS. 19A and 19B demonstrate the ability of a PDL1×41BB bispecific fusion protein to simultaneously bind PDL1 and 41BB. INBRX-105-1 was titrated onto PDL1 expressing K562 cells and 25 nM recombinant 41BB-mFc proteins was added. Bound 41BB was detected using an anti-mouse IgG-Fc specific secondary antibody. FIG. 19A. is a graph showing the binding of INBRX-105-1 to the PDL1 expressing K562 cells. FIG. 19B is a graph showing the binding of recombinant 41BB to INBRX-105-1 on the PDL1 expressing cells.

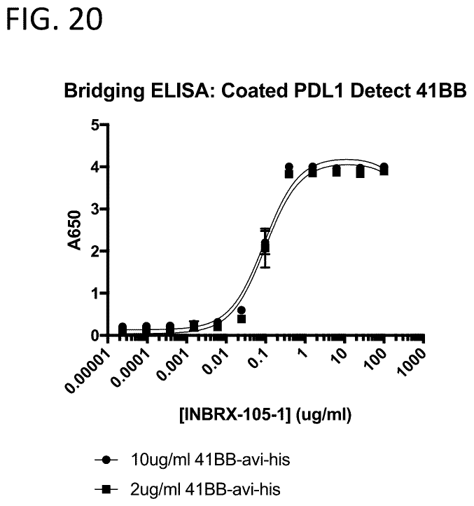

FIG. 20 demonstrates the ability of a PDL1×41BB bispecific fusion protein to simultaneously bind recombinant PDL1 and recombinant 41BB in an ELISA. INBRX-105-1 was titrated on to immobilized (Medisorp plate) recombinant PDL1, subsequently either 2 or l0 g/ml biotinylated-recombinant 41BB (His-tagged) was added. Bound recombinant 41BB was detected via streptavidin-HRP.

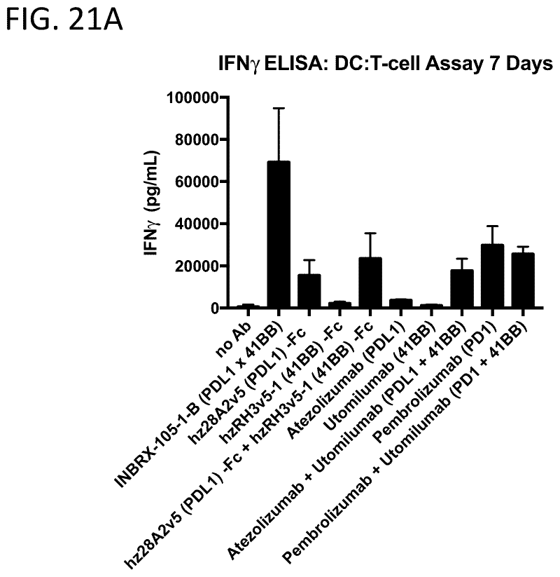

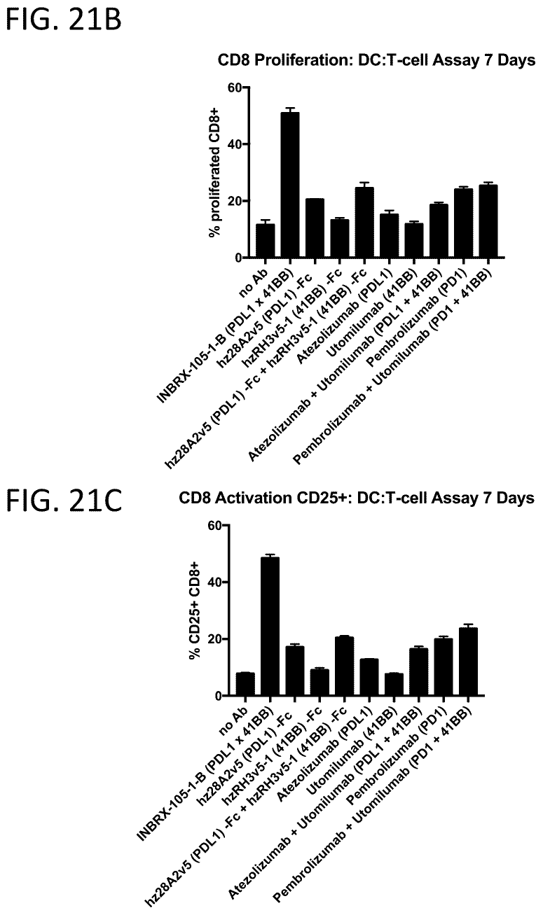

The effect of the PDL1×41BB bispecific fusion proteins to on T-cell activation and proliferation was evaluated. FIGS. 21A, 21B, and 21C demonstrate the effect of a PDL1×41BB bispecific fusion protein (INBRX-105-1) of the present disclosure on T-cell activation and proliferation. Herein an autologous in vitro co-culture system implementing immature DC (iDC) and donor matched T-cells was conducted for 7 days. PDL1⁺ iDC were derived by enriching the monocyte population (EasySep™ Human Monocyte Enrichment Kit, STEMCELL Technologies Inc.) from human donor PBMCs and culturing them in 500 U/ml GM-CSF and 250 U/ml IL-4 for 7 days. Autologous T-cells were enriched at the same time (EasySep™ Human T-cell Enrichment Kit, STEMCELL Technologies Inc.) and cryopreserved until iDC derivation was complete. Enriched T-cells were added to iDC at approximately 20:1 (T-cell:iDC) and co-cultured for at least 7 days in the presence of IL-7. The PDL1×4TBB bispecific, INBRX-105-1, is superior to the monospecific PDL1 sdAb-Fc fusion protein (hz28A2v5-Fc), the 41BB sdAb-Fc fusion protein (hzRH3v5-1-Fc), the combination of the hz28A2v5-Fc and hzRH3v5-1-Fc, the anti-PDL1 antibody Atezolizumab, the anti-41BB antibody, Utomilumab (PF-05082566, disclosed in U.S. Pat. No. 8,337,850), or the anti-PD1 antibody Prembrolizumab, and combinations thereof, at inducing INFγ (FIG. 21A) or mediating CD8+ T-cell proliferation (FIG. 21B) and activation (FIG. 21C). INFγ production in the cell supernatant was monitored using an ELISA and normalized to the standard curve. T-cell proliferation was monitored by flow cytometry using CTV labeling of T-cells. T-cell activation was assessed by the presence of the activation marker CD25 monitored by flow cytometry. Antibodies were used at 10 nM. INBRX-105-1 seemingly augments low level and/or tonic T-cell activation/signaling events that is dampened by the PDL1:PD1 interaction.

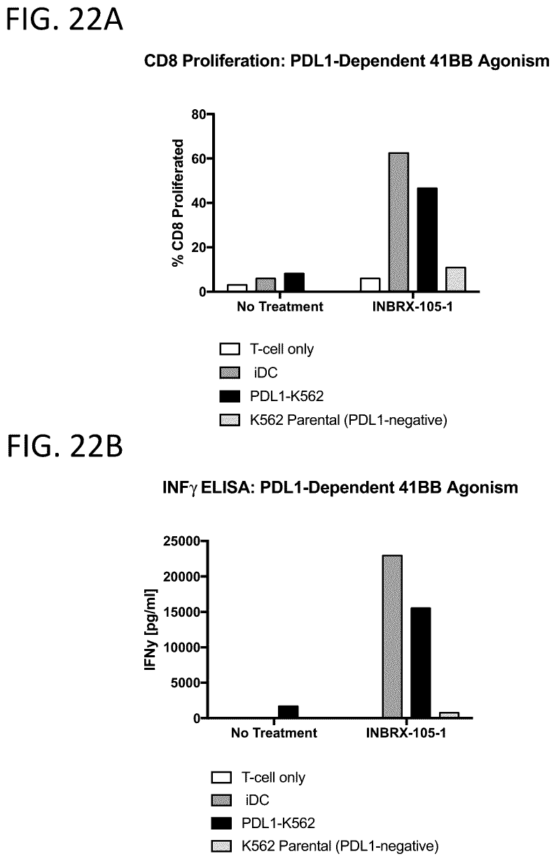

FIGS. 22A and 22B demonstrate PDL1-dependent 41BB agonism mediated by a PDL1×41BB bispecific fusion protein (INBRX-105-1) of the present disclosure. In these studies, T-cells were cultured alone or with autologous immature DCs (iDC, PDL1-expressing), a PDL1-expressing K562 cell line or the parental K562 cell line (PDL1-negative) in the presence or absence of 10 nM INBRX-105-1 for 7 days. CD8⁺ T-cell proliferation (FIG. 22A) was monitored using CTV labeling and INFγ production (FIG. 22B) in the cell supernatant was monitored using an ELISA and normalized to the standard curve.

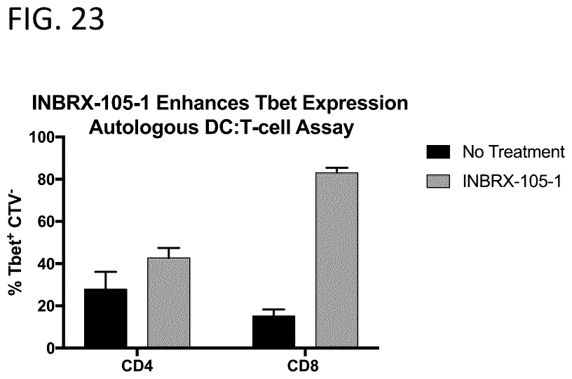

FIG. 23 demonstrates the capacity of a PDL1×41BB bispecific fusion protein (INBRX-105-1) of the present disclosure to enhance the Th1 lineage defining transcription factor, T-bet, expression in T-cell populations. Herein T-cells were co-cultured with autologous immature DCs for 7 days in the presence or absence of INBRX-105-1. T-bet expression was assessed on CD4+ and CD8⁺ T-cell population by flow cytometry via intracellular staining following fixation and permeabilization. INBRX-105-1 has a more dramatic effect on T-bet expression in CD8⁺ T-cells.

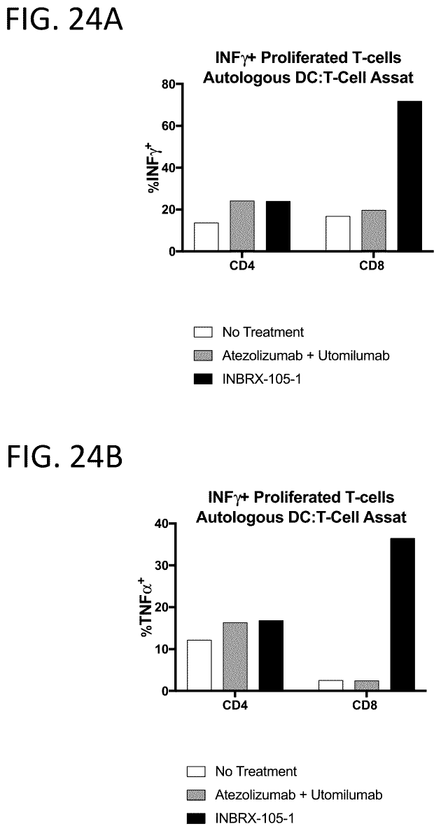

The PDL1×41BB bispecific fusion proteins of the disclosure were compared to various known monospecific antibodies. FIGS. 24A and 24B contrast the capacity of a PDL1×41BB bispecific fusion protein (INBRX-105-1) of the present disclosure and the combination of monospecific antibodies Atezolizumab (anti-PDL1) and Utomilumab (anti-41BB) to induce INFγ (FIG. 24A) or TNFα (FIG. 24B) production from CD4+ or CD8⁺ T-cells. Herein T-cells were co-cultured with autologous immature DCs for 7 days in the presence or absence of INBRX-105-1 or the combination of the monospecific antibodies. INBRX-105-1 is far superior at T-cell co-stimulation compared to monospecific antibodies targeting the same antigens. Cytokine expression was assessed on CD4+ and CD8⁺ T-cell population by flow cytometry via intracellular staining following fixation and permeabilization.

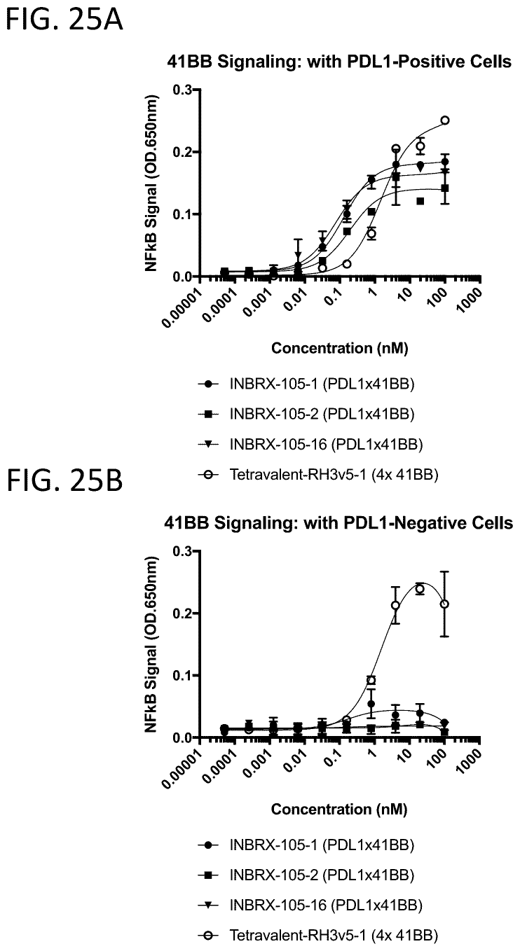

FIGS. 25A and 25B demonstrate the agonistic capacity of a tetravalent 41BB-binding fusion protein and PDL1×41BB bispecific fusion proteins of the present disclosure in the presence of an additional PDL1 positive (FIG. 25A) or negative (FIG. 25B) cell line. Notably only the tetravalent 41BB binding fusion protein is capable of inducing 41BB signaling in the absence of a PDL1 expressing cell line. The bispecific PDL1×41BB fusion proteins (INBRX-105-1, INBRX-105-2 and INBRX-105-16) only induced 41BB signaling when bound to cell surface PDL1 as shown in FIG. 25A. This demonstrates that bivalent engagement of 41BB, as is the case of INBRX-105, is insufficient to effectively cluster and mediate productive 41BB signaling. Engagement of a second cell surface antigen, PDL1 as in the present example, enables further clustering of 41BB and productive signaling. Herein a 41BB-expressing HEK293 NF-kB reporter cell was used and co-incubated with either the PDL1-negative K562 cell line (FIG. 25B) or a stably transfected, PDL1-expressing K562 cell line (FIG. 25A). INBRX-105-1 incorporates the 41BB-targeting sdAb: hzRH3v5-1, INBRX-105-2 incorporates the 41BB-targeting sdAb: hzRH3v5-2 and INBRX-105-16 incorporates the 41BB-targeting sdAb: hzRH3v5-16 and all incorporate the hz28A2v5 PDL1-targeting sdAb. The tetravalent 41BB-targeting fusion protein used herein has the following format comprising hzRH3v5-1-Fc-hzRH3v5-1.

1-47. (canceled)

48. A method of treating cancer comprising administering to a human subject with cancer a polypeptide comprising at least one VHH domain that binds human programmed death ligand 1 (PDL1), wherein at least one VHH domain that binds PDL1 comprises:
(i) a CDR1 comprising an amino acid sequence of SEQ ID NO: 101, a CDR2 comprising an amino acid sequence of SEQ ID NO: 102, and a CDR3 comprising an amino acid sequence of SEQ ID NO: 103;
(ii) a CDR 1 comprising an amino acid sequence of SEQ ID NO: 105, a CDR2 comprising an amino acid sequence of SEQ ID NO: 106, and a CDR comprising an amino acid sequence of SEQ ID NO: 107;
(iii) a CDR 1 comprising an amino acid sequence of SEQ ID NO: 109, a CDR2 comprising an amino acid sequence of SEQ ID NO: 110, and a CDR comprising an amino acid sequence of SEQ ID NO: 111;
(iv) a CDR 1 comprising an amino acid sequence of SEQ ID NO: 101, a CDR2 comprising an amino acid sequence of SEQ ID NO: 110, and a CDR comprising an amino acid sequence of SEQ ID NO: 113;
(v) a CDR 1 comprising an amino acid sequence of SEQ ID NO: 101, a CDR2 comprising an amino acid sequence of SEQ ID NO: 110, and a CDR comprising an amino acid sequence of SEQ ID NO: 115; or
(vi) a CDR 1 comprising an amino acid sequence of SEQ ID NO: 101, a CDR2 comprising an amino acid sequence of SEQ ID NO: 117, and a CDR comprising an amino acid sequence of SEQ ID NO: 118.

49. The method of claim 48, wherein at least one VHH domain that binds PDL1 comprises a CDR1 comprising the amino acid sequence of SEQ ID NO: 105, a CDR2 comprising the amino acid sequence of SEQ ID NO: 106, and a CDR3 comprising the amino acid sequence of SEQ ID NO: 107.

50. The method of claim 48, wherein each VHH domain that binds PDL1 is humanized.

51. The method of claim 48, wherein the polypeptide is monospecific.

52. The method of claim 48, wherein at least one VHH domain that binds PDL1 comprises an amino acid sequence that is at least 95% identical to an amino acid sequence selected from SEQ ID NOs: 100, 104, 108, 112, 114, 116, and 119-124.

54. The method of claim 48, wherein at least one VHH domain that binds PDL1 comprises an amino acid sequence selected from SEQ ID NOs: 100, 104, 108, 112, 114, 116, and 119-124.

55. The method of claim 48, wherein at least one VHH domain that binds PDL1 comprises the amino acid sequence of SEQ ID NO: 124.

56. The method of claim 48, wherein each VHH domain that binds PDL1 comprises the amino acid sequence of SEQ ID NO: 124.

57. The method of claim 48, wherein the isolated polypeptide comprises an Fc region.

58. The method of claim 57, wherein the Fc region comprises an amino acid sequence that is at least 97% or 100% identical to an amino acid sequence selected from SEQ ID NOs: 1-6.

59. The method of claim 48, wherein the cancer is selected from carcinoma, sarcoma, lymphoma, and leukemia.

60. A method of increasing T cell activation and/or proliferation comprising contacting T cells with a polypeptide of claim comprising at least one VHH domain that binds human programmed death ligand 1 (PDL1), wherein at least one VHH domain that binds PDL1 comprises:
(i) a CDR1 comprising an amino acid sequence of SEQ ID NO: 101, a CDR2 comprising an amino acid sequence of SEQ ID NO: 102, and a CDR3 comprising an amino acid sequence of SEQ ID NO: 103;
(ii) a CDR 1 comprising an amino acid sequence of SEQ ID NO: 105, a CDR2 comprising an amino acid sequence of SEQ ID NO: 106, and a CDR comprising an amino acid sequence of SEQ ID NO: 107;
(iii) a CDR 1 comprising an amino acid sequence of SEQ ID NO: 109, a CDR2 comprising an amino acid sequence of SEQ ID NO: 110, and a CDR comprising an amino acid sequence of SEQ ID NO: 111;
(iv) a CDR 1 comprising an amino acid sequence of SEQ ID NO: 101, a CDR2 comprising an amino acid sequence of SEQ ID NO: 110, and a CDR comprising an amino acid sequence of SEQ ID NO: 113;
(v) a CDR 1 comprising an amino acid sequence of SEQ ID NO: 101, a CDR2 comprising an amino acid sequence of SEQ ID NO: 110, and a CDR comprising an amino acid sequence of SEQ ID NO: 115; or
(vi) a CDR 1 comprising an amino acid sequence of SEQ ID NO: 101, a CDR2 comprising an amino acid sequence of SEQ ID NO: 117, and a CDR comprising an amino acid sequence of SEQ ID NO: 118.

61. The method of claim 60, wherein the T cells are CD4⁺ T cells and/or CD8⁺ T cells.

62. The method of claim 60, wherein at least one VHH domain that binds PDL1 comprises a CDR1 comprising the amino acid sequence of SEQ ID NO: 105, a CDR2 comprising the amino acid sequence of SEQ ID NO: 106, and a CDR3 comprising the amino acid sequence of SEQ ID NO: 107.

63. The method of claim 60, wherein each VHH domain that binds PDL1 is humanized.

64. The method of claim 60, wherein the polypeptide is monospecific.

65. The method of claim 60, wherein at least one VHH domain that binds PDL1 comprises an amino acid sequence that is at least 95% identical to an amino acid sequence selected from SEQ ID NOs: 100, 104, 108, 112, 114, 116, and 119-124.

66. The method of claim 60, wherein at least one VHH domain that binds PDL1 comprises an amino acid sequence selected from SEQ ID NOs: 100, 104, 108, 112, 114, 116, and 119-124.

67. The method of claim 60, wherein at least one VHH domain that binds PDL1 comprises the amino acid sequence of SEQ ID NO: 124.

68. The method of claim 60, wherein each VHH domain that binds PDL1 comprises the amino acid sequence of SEQ ID NO: 124.

69. The method of claim 60, wherein the isolated polypeptide comprises an Fc region.

70. The method of claim 69, wherein the Fc region comprises an amino acid sequence that is at least 97% or 100% identical to an amino acid sequence selected from SEQ ID NOs: 1-6.
